(19) United States
(12) Patent Application Publication
(10) Pub. No.: US 2026/0151119 A1
(43) Pub. Date: Jun. 4, 2026

(54) TRANSPERINEAL PROSTATE BIOPSY GUIDE SYSTEM

(71) Applicants: BOSTON SCIENTIFIC MEDICAL DEVICE LIMITED, Galway (IE); BOSTON SCIENTIFIC SCIMED, INC., Maple Grove, MN (US)

(72) Inventors: SUSHIL KUMAR, Muzaffarpur (IN); AMIT KUMAR, Noida (IN); PRIYANSHU GUPTA, Ujjain (IN); UMESH KUMAR BADAYA, Bandikui (IN); VIVEK SHARMA, Kanpur (IN); JONATHAN J. LUND, Glencoe, MN (US); PATRICK NICOLAS, Mansfield, MA (US)

(73) Assignees: BOSTON SCIENTIFIC MEDICAL DEVICE LIMITED, Galway (IE); BOSTON SCIENTIFIC SCIMED, INC., Maple Grove, MN (US)

(21) Appl. No.: 19/408,056

(22) Filed: Dec. 3, 2025

Related U.S. Application Data
(60) Provisional application No. 63/727,955, filed on Dec. 4, 2024.

(51) Int. Cl. A61B 10/02; A61B 8/00; A61B 8/12

(52) U.S. Cl. CPC A61B 10/0241; A61B 8/12; A61B 8/4218

(57) ABSTRACT

A prostate biopsy guide system may comprise a horizontal slide member, a vertical support movably coupled to the horizontal slide member, a vertical slide member movably coupled to the vertical support, a needle mount coupled to the vertical slide member, and a locking mechanism coupled to the vertical slide member. The system may allow precise positioning and control of a biopsy needle for transperineal prostate biopsy procedures. The locking mechanism may enable selective locking of vertical movement and swivel movement in different configurations. The horizontal slide member may include rails and a crossbar for mounting to an ultrasound probe. The vertical support and slide member may allow controlled vertical and angular adjustment of the needle mount.

### CROSS-REFERENCE TO RELATED APPLICATIONS

The application claims the benefit of U.S. Provisional Patent Application Ser. No. 63/727,955, filed on Dec. 4, 2024, the disclosure of which is incorporated herein by reference.

### TECHNICAL FIELD

The disclosure is directed to biopsy guide systems. More particularly, the disclosure is directed to biopsy guide system for transperineal prostate biopsy.

### BACKGROUND

Prostate biopsy is usually recommended by a urologist when the results from a prostate-specific antigen (PSA) blood test is reported abnormal or a digital rectal exam feels suspicious. Prostate tissue biopsy may be taken under local, spinal, or general anesthesia and guided by medical ultrasound imaging using a transrectal or transperineal approach. In a transrectal approach, a biopsy needle is placed through the rectal wall and into the prostate. A transperineal approach uses a transrectal ultrasound probe for imaging and allows for multiple biopsies through the perineum, the region between the scrotum and the anus. This approach greatly reduces the risk of infection as compared to the transrectal approach and allows for biopsy of the entire prostate gland, including anterior edges of the prostate. Of the known biopsy systems and methods, each has certain advantages and disadvantages. Accordingly, there is an ongoing need to provide alternative systems and methods for prostate biopsy.

### SUMMARY

The disclosure is directed to several alternative designs, materials and methods of manufacturing medical device structures and assemblies.

In an example, a prostate biopsy guide system may include a horizontal slide member, a vertical support movably coupled to the horizontal slide member, a vertical slide member movably coupled to the vertical support, a needle mount coupled to the vertical slide member, and a locking mechanism coupled to the vertical slide member.

Alternatively or additionally to any of the examples above, in another example, the horizontal slide member may include a first rail, a second rail laterally spaced from the first rail, and a crossbar extending between the first rail and the second rail.

Alternatively or additionally to any of the examples above, in another example, the horizontal slide member may further include a probe mount positioned adjacent to a proximal region of the horizontal slide member and extending between the first rail and the second rail.

Alternatively or additionally to any of the examples above, in another example, the vertical support may include a proximal face, a distal face axially spaced from the proximal face, and a U-shaped bracket connecting the proximal face and the distal face.

Alternatively or additionally to any of the examples above, in another example, the vertical support may further include a first laterally inwardly extending wall and a second laterally inwardly extending wall.

Alternatively or additionally to any of the examples above, in another example, the vertical slide member may be configured to move along the first and second laterally inwardly extending walls.

Alternatively or additionally to any of the examples above, in another example, the system may further include an actuatable end cover coupled to the vertical support.

Alternatively or additionally to any of the examples above, in another example, the actuatable end cover may be vertically displaceable relative to the vertical support to selectively secure the vertical support relative to the horizontal slide member.

Alternatively or additionally to any of the examples above, in another example, the vertical slide member may include a proximal region configured to be coupled to the locking mechanism and a distal region configured to be movably coupled to the vertical support.

Alternatively or additionally to any of the examples above, in another example, the distal region of the vertical slide member may include an elongated central aperture.

Alternatively or additionally to any of the examples above, in another example, the needle mount may include a tubular body defining a lumen for receiving a portion of a guide needle therethrough.

Alternatively or additionally to any of the examples above, in another example, the locking mechanism may be movable between a first configuration in which both vertical movement of the vertical slide member and swivel movement of the needle mount are prevented, a second configuration in which vertical movement of the vertical slide member is prevented while allowing swivel movement of the needle mount, and a third configuration in which both vertical movement of the vertical slide member and swivel movement of the needle mount are allowed.

Alternatively or additionally to any of the examples above, in another example, the locking mechanism may include a handle, a first connection member extending from the handle, a second connection member extending from the handle, a first swivel lock cam member coupled to the first connection member, a second swivel lock cam member coupled to the second connection member, a first vertical lock cam member coupled to the first connection member, and a second vertical lock cam member coupled to the second connection member.

Alternatively or additionally to any of the examples above, in another example, the first and second swivel lock cam members may be configured to selectively engage mounting members of the needle mount to prevent swivel movement of the needle mount.

Alternatively or additionally to any of the examples above, in another example, the first and second vertical lock cam members may be configured to selectively engage a proximal face of the vertical support to prevent vertical movement of the vertical slide member.

In an example, a prostate biopsy guide system may include a horizontal slide member, a vertical support movably coupled to the horizontal slide member, a vertical slide member movably coupled to the vertical support, a needle mount coupled to the vertical slide member, and a locking mechanism coupled to the vertical slide member.

Alternatively or additionally to any of the examples above, in another example, the horizontal slide member may include a first rail, a second rail laterally spaced from the first rail, and a crossbar extending between the first rail and the second rail.

Alternatively or additionally to any of the examples above, in another example, the crossbar may include a concave lower surface configured to conform to an outer surface of an ultrasound probe.

Alternatively or additionally to any of the examples above, in another example, the horizontal slide member may further include a probe mount positioned adjacent to a proximal region of the horizontal slide member and extending between the first rail and the second rail.

Alternatively or additionally to any of the examples above, in another example, the probe mount may include a concave lower surface configured to conform to an outer surface of an ultrasound probe.

Alternatively or additionally to any of the examples above, in another example, the probe mount may include a first opening and a second opening, each configured to receive an end of a strap.

Alternatively or additionally to any of the examples above, in another example, the vertical support may comprise a proximal face, a distal face axially spaced from the proximal face, and a U-shaped bracket connecting the proximal face and the distal face.

Alternatively or additionally to any of the examples above, in another example, the vertical support may further comprise a first laterally inwardly extending wall and a second laterally inwardly extending wall.

Alternatively or additionally to any of the examples above, in another example, the vertical slide member may be configured to move along the first and second laterally inwardly extending walls.

Alternatively or additionally to any of the examples above, in another example, the prostate biopsy guide system may further comprise an actuatable end cover coupled to the vertical support.

Alternatively or additionally to any of the examples above, in another example, the actuatable end cover may be vertically displaceable relative to the vertical support to selectively secure the vertical support relative to the horizontal slide member.

Alternatively or additionally to any of the examples above, in another example, the vertical slide member may comprise a proximal region configured to be coupled to the locking mechanism and a distal region configured to be movably coupled to the vertical support.

Alternatively or additionally to any of the examples above, in another example, the distal region of the vertical slide member may include an elongated central aperture.

Alternatively or additionally to any of the examples above, in another example, the needle mount may comprise a tubular body defining a lumen for receiving a portion of a guide needle therethrough.

Alternatively or additionally to any of the examples above, in another example, the locking mechanism may be movable between a first configuration in which both vertical movement of the vertical slide member and swivel movement of the needle mount are prevented, a second configuration in which vertical movement of the vertical slide member is prevented while allowing swivel movement of the needle mount, and a third configuration in which both vertical movement of the vertical slide member and swivel movement of the needle mount are allowed.

In an example, a prostate biopsy guide system may include a horizontal slide member configured to be coupled to an ultrasound probe, a vertical support movably coupled to the horizontal slide member, a vertical slide member movably coupled to the vertical support, a needle mount pivotably coupled to the vertical slide member, and a locking mechanism coupled to the vertical slide member and configured to selectively lock a vertical position of the vertical slide member and an angular position of the needle mount.

Alternatively or additionally to any of the examples above, in another example, the locking mechanism may include a handle, a first connection member extending from the handle, a second connection member extending from the handle, a first swivel lock cam member coupled to the first connection member, a second swivel lock cam member coupled to the second connection member, a first vertical lock cam member coupled to the first connection member, and a second vertical lock cam member coupled to the second connection member.

Alternatively or additionally to any of the examples above, in another example, the first and second swivel lock cam members may be configured to selectively engage mounting members of the needle mount to prevent swivel movement of the needle mount.

Alternatively or additionally to any of the examples above, in another example, the first and second vertical lock cam members may be configured to selectively engage a proximal face of the vertical support to prevent vertical movement of the vertical slide member.

In an example, a prostate biopsy guide system may include a horizontal slide member configured to be coupled to an ultrasound probe, a vertical support movably coupled to the horizontal slide member, a vertical slide member movably coupled to the vertical support, a needle mount pivotably coupled to the vertical slide member, and means for selectively locking a vertical position of the vertical slide member and an angular position of the needle mount.

In an example, a prostate biopsy guide system may comprise a horizontal support member, a vertical housing coupled to the horizontal support member, a first gear assembly comprising at least one bevel gear, a second gear assembly comprising at least one bevel gear, a third gear assembly comprising at least one bevel gear, a needle mount member coupled to at least one of the gear assemblies, and control knobs operatively coupled to each of the first gear assembly, the second gear assembly, and the third gear assembly.

Alternatively or additionally to any of the examples above, in another example, the first gear assembly may control vertical movement of the needle mount member.

Alternatively or additionally to any of the examples above, in another example, the second gear assembly may control angular movement of the needle mount member.

Alternatively or additionally to any of the examples above, in another example, the third gear assembly may control horizontal movement of the vertical housing relative to the horizontal support member.

Alternatively or additionally to any of the examples above, in another example, each gear assembly may comprise an elongate shaft coupled between its respective at least one bevel gear and control knob.

Alternatively or additionally to any of the examples above, in another example, the at least one bevel gear is configured to translate rotational movement between perpendicular axes.

In another example, a prostate biopsy guide system may comprise a vertical housing, a needle mount member movably coupled to the vertical housing, a first push button assembly, a second push button assembly, and at least one floating locking member operatively coupled to at least one of the push button assemblies. Actuation of the push button assemblies may selectively control vertical and angular movement of the needle mount member.

Alternatively or additionally to any of the examples above, in another example, the floating locking member may comprise a body portion with an opening configured to receive a mounting member of the needle mount member.

Alternatively or additionally to any of the examples above, in another example, the floating locking member may include at least one securement member configured to engage with a plurality of pins.

Alternatively or additionally to any of the examples above, in another example, the system may further comprise a biasing member positioned between the needle mount member and the floating locking member.

Alternatively or additionally to any of the examples above, in another example, each push button assembly may include a stem portion configured to engage with a lateral side opening of the vertical housing.

Alternatively or additionally to any of the examples above, in another example, the push button assemblies may be independently actuatable.

In another example, a prostate biopsy guide system may comprise a vertical housing, a needle actuation assembly movably coupled to the vertical housing, a first lead screw assembly configured to control vertical movement, a second lead screw assembly configured to control angular movement, and a third lead screw assembly configured to control horizontal movement.

Alternatively or additionally to any of the examples above, in another example, at least one lead screw assembly may comprise a helically threaded shaft.

Alternatively or additionally to any of the examples above, in another example, the first lead screw assembly may engage with a vertical travel shaft.

Alternatively or additionally to any of the examples above, in another example, the second lead screw assembly may engage with a swivel shaft.

Alternatively or additionally to any of the examples above, in another example, the third lead screw assembly may engage with a horizontal travel shaft.

The above summary of some example embodiments is not intended to describe each disclosed embodiment or every implementation of the disclosure.

### BRIEF DESCRIPTION OF THE DRAWINGS

The disclosure may be more completely understood in consideration of the following detailed description of various embodiments in connection with the accompanying drawings, in which:

FIG. 1 is a perspective view of an illustrative prostate biopsy guide system in a first configuration.

FIG. 2 is a perspective view of the illustrative prostate biopsy guide system of FIG. in a second configuration.

FIG. 3A is a perspective view of an illustrative vertical support.

FIG. 3B is a front view of the illustrative vertical support.

FIG. 4A is a perspective view of an illustrative actuatable end cover.

FIG. 4B is a front view of the illustrative actuatable end cover.

FIG. 5 is a perspective view of the prostate biopsy guide system with the actuatable end cover in a second or unlocked configuration.

FIG. 6A is a perspective view of an illustrative vertical slide member.

FIG. 6B is a front view of the illustrative vertical slide member.

FIG. 7A is a perspective view of an illustrative actuatable locking mechanism.

FIG. 7B is a right-side view of the illustrative actuatable locking mechanism.

FIG. 8 is a perspective view of an illustrative needle mount.

FIG. 9 is a perspective view of an illustrative guide needle.

FIG. 10 is a partial side view of the prostate biopsy guide system with the locking mechanism in the first configuration.

FIG. 11 is a partial cross-sectional view of the prostate biopsy guide system, taken at line 11-11 of FIG. 10.

FIG. 12 is a partial side view of the prostate biopsy guide system with the locking mechanism in the second configuration.

FIG. 13 is a partial cross-sectional view of the prostate biopsy guide system, taken at line 13-13 of FIG. 12.

FIG. 14 is a partial side view of the prostate biopsy guide system with the locking mechanism in the third configuration.

FIG. 15 is a partial cross-sectional view of the prostate biopsy guide system, taken at line 15-15 of FIG. 14.

FIG. 16 is a perspective view of another illustrative prostate biopsy guide system in a first configuration.

FIG. 17 is a bottom perspective view of the horizontal slide member of the prostate biopsy guide system of FIG. 16.

FIG. 18A is a perspective view of an illustrative vertical housing of the prostate biopsy guide system of FIG. 16.

FIG. 18B is a front view of the illustrative vertical housing of the prostate biopsy guide system of FIG. 16.

FIG. 19 is a cross-sectional view of the illustrative vertical housing, taken at line 19-19 of FIG. 18A.

FIG. 20 is a perspective view of an illustrative needle actuation assembly of the prostate biopsy guide system of FIG. 16.

FIG. 21 is a perspective view of the illustrative push buttons of the prostate biopsy guide system of FIG. 16.

FIG. 22 is a perspective view of the illustrative floating locking members of the prostate biopsy guide system of FIG. 16.

FIG. 23A is a perspective view of the illustrative needle mount member of the prostate biopsy guide system of FIG. 16.

FIG. 23B is a top view of the needle mount member of the prostate biopsy guide system of FIG. 16 with a guide needle secured relative to the needle mount member.

FIG. 24 is a partial cross-sectional view of the vertical housing of the prostate biopsy guide system of FIG. 16 with the locking and actuation mechanism in a first or fully locked configuration.

FIG. 25 is a perspective view of the partial cross-sectional view of FIG. 24.

FIG. 26 is a partial cross-sectional view of the vertical housing of the prostate biopsy guide system of FIG. 16 with the locking and actuation mechanism in a second or partially locked configuration.

FIG. 27 is a schematic side view of the of the guide system of FIG. 16 illustrating the pivotable arrangement of the guide needle and the needle mount member.

FIG. 28 is a partial cross-sectional view of the vertical housing of the guide system of FIG. 16 with the locking and actuation mechanism in a third or fully unlocked configuration.

FIG. 29A is a side view of another illustrative push button that may be used with the prostate biopsy guide system of FIG. 16.

FIG. 29B is a perspective view of the illustrative push button of FIG. 29A.

FIG. 30 is a perspective view of another illustrative prostate biopsy guide system in a first configuration.

FIG. 31A is a perspective view of an illustrative vertical support of the prostate biopsy guide system of FIG. 30.

FIG. 31B is a front view of the illustrative vertical support of FIG. 31A.

FIG. 31C is a cross-sectional view of the vertical support taken at line 31C-31C of FIG. 31A.

FIG. 32 is a cross-sectional view of the prostate biopsy guide system of FIG. 30.

FIG. 33 is a cross-sectional view of the prostate biopsy guide system of FIG. 30 with a locking mechanism in a second or unlocked configuration.

FIG. 34A is a perspective view of an illustrative needle actuation assembly for use in the prostate biopsy guide system of FIG. 30.

FIG. 34B is a top view of the illustrative needle actuation assembly of FIG. 34A.

FIG. 35A is an exploded front perspective view of the needle actuation assembly of FIG. 34A.

FIG. 35B is an exploded back view of the needle actuation assembly of FIG. 34A.

FIG. 36 is a top view of the needle actuation assembly with a gear assembly housing removed.

FIG. 37 is a cross-sectional view of the needle actuation assembly taken at line 37-37 of FIG. 34A.

FIG. 38 is an exploded perspective view of a gear assembly of the needle actuation assembly.

FIG. 39 is a cross-sectional view of the needle actuation assembly of FIG. 34A with the needle mount member and the gear assembly in an unlocked configuration.

FIG. 40 is a cross-sectional view of the guide system of FIG. 30 with the push buttons in an unlocked configuration.

FIG. 41 is a perspective view of another illustrative prostate biopsy guide system in a first configuration.

FIG. 42 is a perspective view of an illustrative needle actuation assembly for use in the prostate biopsy guide system of FIG. 41.

FIG. 43 is a partial cross-sectional view of the prostate biopsy guide system of FIG. 41, taken at line 43-43 of FIG. 41.

FIG. 44 is a left side view of the prostate biopsy guide system of FIG. 41.

FIG. 45 is a perspective view of another illustrative prostate biopsy guide system in a first configuration.

FIG. 46A is a perspective view of an illustrative vertical support of the prostate biopsy guide system of FIG. 45.

FIG. 46B is a partial cross-sectional view of the illustrative vertical support taken at line 46B-46B of FIG. 46A.

FIG. 47A is a perspective view of an illustrative needle actuation assembly for use in the prostate biopsy guide system of FIG. 45.

FIG. 47B is a perspective view of the illustrative needle actuation assembly with a housing of the gear assembly member removed.

FIG. 47C is a cross-sectional view of the needle actuation assembly, taken at line 47C-47C of FIG. 47A.

FIG. 48A is a proximal or front view of the gear assembly member of the needle actuation assembly of FIG. 47A.

FIG. 48B which is a top view of the gear assembly member.

FIG. 49 is an exploded view of an illustrative gear assembly of the needle actuation assembly of FIG. 47A.

FIG. 50 is a perspective partial cross-sectional view of the needle actuation assembly of FIG. 47A in a first configuration.

FIG. 51 is a perspective view of another illustrative prostate biopsy guide system in a first configuration.

FIG. 52A is a perspective view of an illustrative horizontal support member of the prostate biopsy guide system of FIG. 51.

FIG. 52B is an exploded perspective view of the horizontal support member of FIG. 52A.

FIG. 53 is a perspective view of an illustrative vertical housing of the prostate biopsy guide system of FIG. 51.

FIG. 54 is a perspective view of the left and right end covers of the prostate biopsy guide system of FIG. 51.

FIG. 55 is a perspective view of the illustrative vertical housing of the prostate biopsy guide system of FIG. 51.

FIG. 56 is a perspective view of an illustrative gear and actuation assembly of the prostate biopsy guide system of FIG. 51.

FIG. 57 is a perspective view of a first gear and actuation assembly and a support shaft disposed within a bottom cover of the proximal housing of the prostate biopsy guide system of FIG. 51.

FIG. 58 is a perspective view of a second and third gear and actuation assembly of the prostate biopsy guide system of FIG. 51.

FIG. 59 is a side view of the prostate biopsy guide system of FIG. 51 with an end cover removed.

FIG. 60 is a cross-sectional view of the prostate biopsy guide system of FIG. 51, taken at line 60-60 of FIG. 59.

FIG. 61A is a perspective view of the fourth gear assembly of the prostate biopsy guide system of FIG. 51 assembled with the needle mount member and a vertical slide assembly.

FIG. 61B is an exploded perspective view of the fourth gear assembly of FIG. 61A, the needle mount member, and the vertical slide assembly.

While the disclosure is amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

Prostate biopsy is usually recommended by a urologist when the results from a prostate-specific antigen (PSA) blood test are reported abnormal or a digital rectal exam feels suspicious. Prostate tissue biopsy may be taken under local, spinal, or general anesthesia and guided by medical ultrasound imaging using a transrectal or transperineal approach. In a transrectal approach, a biopsy needle is placed through the rectal wall and into the prostate. A transperineal approach uses a transrectal ultrasound probe for imaging and allows for multiple biopsies through the perineum, the region between the scrotum and the anus. This approach greatly reduces the risk of infection as compared to a transrectal approach and allows for biopsy of the entire prostate gland, including anterior edges of the prostate.

In a “freehand” transperineal biopsy approach, the user may maneuver the ultrasound probe freely, as in a transrectal approach. The region of interest may be accessed directly with the biopsy needle aided by an introducer needle. Multiple punctures may be required if a biopsy is taken directly with a biopsy needle without the use of an introducer needle using a typical grid guide. With an introducer needle two single punctures may be made on either side of the perineum about 1.5 centimeters (cm) above the anal border and 2 cm lateral, thereby removing the need for multiple skin punctures and allows for local anesthetic to be localized to the limited points of puncture. The number of cores taken using this technique may vary, with some physicians obtaining a smaller number of cores similar to a transrectal biopsy and others following the Ginsberg protocol.

The present freehand approach may present some challenges. For example, multiple punctures in the perineum may be required if a biopsy needle is used without an introducer needle for precise access to an intended location. With an introducer needle, the deployment of the biopsy needle through the perineum can be challenging, as the angular orientation of the introducer needle must be ensured throughout the travel length to achieve precise access to the region of interest for the biopsy. Further, the position of the introducer needle with respect to the ultrasound probe may need to be changed to gain access to different sections of the prostate for biopsy. Precise repositioning and stability of the introducer needle may become challenging for the physician during biopsy. In some cases, it may be difficult to align an introducer needle and a biopsy needle in the sagittal plane during biopsy core sampling. Finally, deflection of an introducer needle may require manipulation with the ultrasound probe and the biopsy needle to take anterior, posterior, and lateral samples. The present disclosure is directed towards systems and methods for performing prostate biopsies which alleviate some of the above challenges.

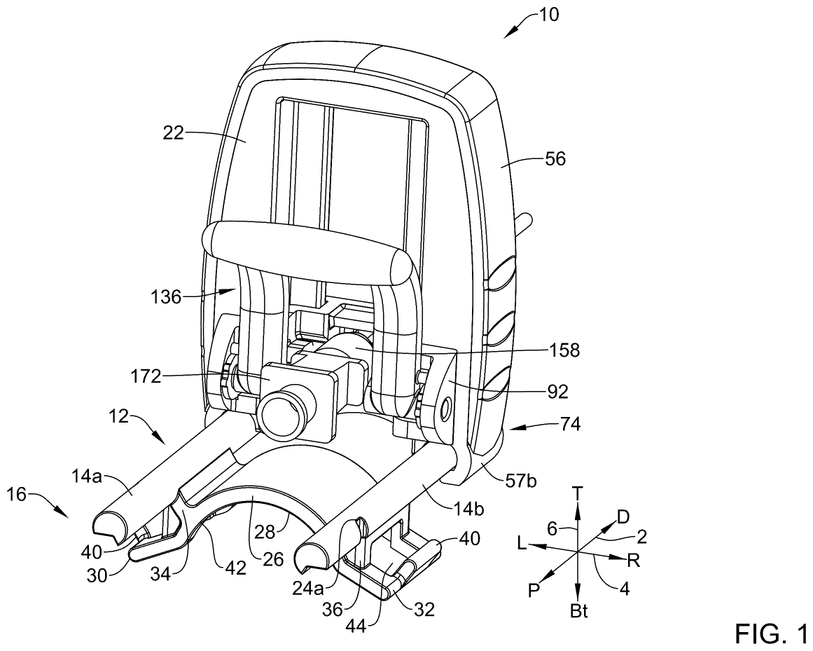

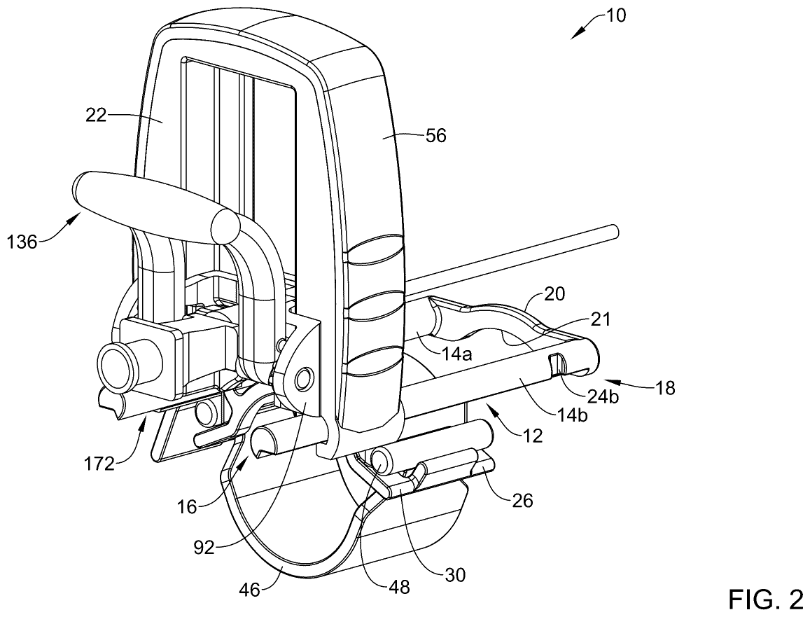

FIG. 1 is a perspective view an illustrative prostate biopsy guide system 10 in a first configuration. FIG. 2 is a perspective view of the illustrative guide system 10 in a second configuration. While the guide system 10 is described with respect to prostate biopsy, it is contemplated that the guide system 10 may be used in other procedures. In one illustrative example, the guide system 10 may be used to facilitate the injection of a spacing material, such as between the rectum and the prostate to prevent damage during radiation therapy. One illustrative system for injecting materials into the space between the rectum and the prostate is the SpaceOAR Vue™ System available from Boston Scientific Corporation, Marlborough, MA, USA. The guide system 10 may be configured to allow for horizontal, vertical, and angular movement of the guide or introducer needle 172 and the biopsy needle (not explicitly shown) while also fixing the biopsy needle in a particular configuration. The guide system 10 may be configured to mount to an ultrasound probe (not explicitly shown) such that the guide system 10 is in a generally fixed position relative to the ultrasound probe for a duration of the procedure. As used herein, movement in the horizontal direction or relational positioning in a direction generally parallel to axis 2, will be described as axial movement or an axial direction with relational positioning using proximal (P) or front and distal (d) or back; movement or relational positioning in a direction generally parallel to axis 4 will be described as lateral movement or a lateral direction with relational positioning using left (L) and right (R); and movement or relational positioning in a direction generally parallel to axis 6 will be described as vertical movement or a vertical direction with relational positioning using top (T) or up and bottom (Bt) or down. The use of the terms “proximal”, “distal”, “front,” “back”, “left”, “right”, “top”, “bottom”, “up”, and “down” are not intended to limit the guide system 10 to a particular orientation, but rather facilitate discussion of relative orientation.

Generally, the guide system 10 may be configured to control a horizontal position, a vertical position, and/or an angle of a guide needle 172. The guide system 10 may be configured to lock the guide needle 172 in a desired orientation independent of and relative to an ultrasound probe. Further, the vertical and/or angular position may be held as the guide needle 172 is moved towards the patient's body and/or away from the patient's body. While not explicitly shown, a biopsy needle may be advanced through the guide needle 172 to obtain the biopsy sample(s). The guide system 10 may include a horizontal slide member 12, a vertical support 22, a vertical slide member 92, a locking mechanism 136, and a needle mount 158, among other features. A portion of the needle mount 158 may extend through and be pivotable relative to a portion of the vertical slide member 92. The vertical slide member 92 may be configured to move up and down along the vertical support 22 to control a vertical position of the needle mount 158 (and thus the guide needle 172). The locking mechanism 136 may be configured to selectively lock a vertical position of the vertical slide member 92 and an angular position of the needle mount 158. The vertical support 22 may be moved along the horizontal slide member 12 to move the guide needle 172 towards and/or way from the patient. The vertical support 22 may be selectively secured to the horizontal slide member 12 to lock the vertical support 22 in a desired axial position.

The horizontal slide member 12 may include a first, or left, rail 14a and a second, or right, rail 14b. The left and right rails 14a, 14b may be laterally spaced and extend generally parallel to one another. The left and right rails 14a, 14b may extend from a first or proximal region 16 of the horizontal slide member 12 to a second or distal region 18 (see, for example, FIG. 2) of the horizontal slide member 12. A third rail or crossbar 20 may extend between the left and right rails 14a, 14b adjacent the distal region 18 thereof. The crossbar 20 may hold the left and right rails 14a, 14b in a laterally spaced configuration and limit horizontal (proximal to distal) movement of a vertical frame or vertical support 22. The crossbar 20 may include a concave lower or bottom surface 21 configured to conform to an outer surface of the ultrasound probe. For example, the concave lower surface 21 may rest on an outer surface of the ultrasound probe. In some embodiments, the crossbar 20 may be formed as single monolithic structure with the left and right rails 14a, 14b. In other embodiments, the crossbar 20 may be formed as a separate component from the first and/or second rails 14a, 14b and subsequently coupled with the first and/or second rails 14a, 14b.

The right rail 14b may include a first recess or notch 24a positioned adjacent to the proximal region 16 and a second recess or notch 24b positioned adjacent to the distal region 18. The first and second recesses 24a, 24b may be formed in a laterally outward edge thereof. While not explicitly shown, the left rail 14a may also include a first recess positioned adjacent to the proximal region 16 and a second recess or notch positioned adjacent to the distal region 18. The recesses in the left rail 14a may be positioned at a same axial location as the corresponding recesses 24a, 24b in the right rail 14b. The recesses 24a, 24b may be positions where the vertical frame 22 may be locked into a particular position or configuration. While FIGS. 1 and 2 illustrate two axial locations (e.g., two recesses 24a, 24b and the corresponding recesses in the left rail 14a) where the vertical frame 22 may be locked into position, the left and right rails 14a, 14b may include more than two axial locations for securing the vertical frame 22 relative to the horizontal slide member 12. In some examples, the vertical support 22 may be secured relative to the horizontal slide member 12 such that the axial location of the vertical support 22 is infinitely adjustable.

The horizontal slide member 12 may further include a probe mount 26 positioned adjacent to the proximal region 16 and extending between the left and right rails 14a, 14b. In some embodiments, the probe mount 26 may be formed as single monolithic structure with the left and right rails 14a, 14b. In other embodiments, the probe mount 26 may be formed as a separate component from the left and/or right rails 14a, 14b and subsequently coupled with the left and/or right rails 14a, 14b. The probe mount 26 may include a concave lower or bottom surface 28 configured to conform to an outer surface of the ultrasound probe. For example, the concave lower surface 28 may rest on an outer surface of the ultrasound probe. Said differently, the probe mount 26 may include an arch or a curve extending between the left and right rails 14a, 14b. The probe mount 26 may extend from a first or left lateral end 30 to a second or right lateral end 32. The probe mount 26 may be connected to the left rail 14a via a first or left connection member 34 extending between a curved intermediate region of the probe mount 26 and the left rail 14a. Similarly, the probe mount 26 may be connected to the right rail 14b via a second or right connection member 36 extending between a curved intermediate region of the probe mount 26 and the right rail 14b.

The left and right lateral ends 30, 32 of the probe mount 26 may extend laterally beyond the left and right rails 14a, 14b, respectively. Each of the left and right lateral ends 30, 32 may include left and right raised edges 38, 40, respectively, extending at a non-parallel angle to the left and right lateral ends 30, 32.

The probe mount 26 may include a first or left opening or aperture 42 positioned adjacent to the left lateral end 30 and extending through a thickness thereof. The first opening 42 may be positioned between the left raised edge 38 and the left connection member 34. The probe mount 26 may further include a second or right opening or aperture 44 positioned adjacent to the right lateral end 32 and extending through a thickness thereof. The second opening 44 may be positioned between the right raised edge 40 and the right connection member 36. The first and second openings 42, 44 may each be configured to receive an end 48 of a strap 46 (see, for example, FIG. 2). The ends 48 of the strap 46 may be sized and shaped to allow the ends 48 to be inserted at an angle through the openings 42, 44 in a bottom to top manner. Once the ends are through the openings 42, 44, the first end (not explicitly shown) may be positioned between the left connection member 34 and the left raised edge 38. The left connection member 34 and the left raised edge 38 may prevent lateral movement of the first end of the strap 46. The end of the strap 46 may have a length greater than a length of the first opening 42 such that the first end of the strap 46 may rest on the probe mount 26. Similarly, the second end 48 may be positioned between the right connection member 36 and the right raised edge 40. The right connection member 36 and the right raised edge 40 may prevent lateral movement of the second end 48 of the strap 46. The second end 48 of the strap 46 may have an axial length greater than a length of the second opening 44 such that the second end 48 of the strap 46 may rest on the probe mount 26. When both ends of the strap 46 are secured between the respective connection members 34, 36 and raised edges 38, 40, the strap 46 may be configured to secure an ultrasound probe to the probe mount 26. For example, the ultrasound probe may be positioned between the concave lower surface 28 and a surface of the strap 46 to releasably secure the ultrasound probe to the guide system 10. The strap 46 may be uncoupled from one or both of the openings 42, 44 by angling the respective end and lowering it through the opening 42, 44. It is contemplated that the guide system 10 may include additional or alternative securement mechanisms for securing the ultrasound probe relative to the guide system 10.

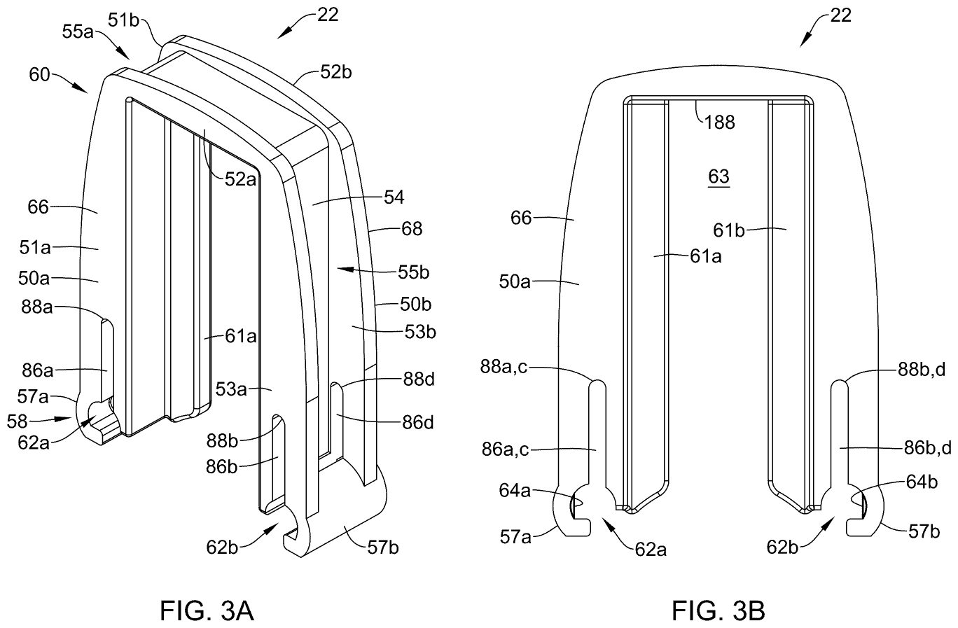

The vertical support 22 may be configured to be axially displaced along the horizontal slide member 12. FIG. 3A is a perspective view of an illustrative vertical support 22 and FIG. 3B is a front view of the illustrative vertical support 22. The vertical support 22 may extend from a bottom end region 58 to a top end region 60. The vertical support 22 may be formed as a single monolithic structure or as two or more components coupled together. The vertical support 22 may have a first or proximal face or plate 50a and a second or distal face or plate 50b. Each of the proximal face 50a and the distal face 50b may have a generally inverted “U” shape having a first or left arm 51a, 51b a second or right arm 53a, 53b laterally spaced from the left arm 51a, 51b, and an interconnecting arm 52a, 52b extending between a top portion 60 of the left arms 51a, 51b and the right arms 53a, 53b. The left arms 51a, 53a and right arms 51b, 53b may extend generally parallel to one another. The interconnecting arms 52a, 52b may extend generally orthogonal to the left arms 51a, 51b and the right arms 53a, 53b.

The proximal and distal faces 50a, 50b may be axially spaced and connected by a generally U-shaped member or bracket 54. The proximal and distal faces 50a, 50b may extend laterally beyond the U-shaped bracket 54 in both the left and right directions to define a first recess 55a extending along a first or left side of the vertical support 22 and a second recess 55b extending along a second or right side of the vertical support 22. The first and second recesses 55a, 55b may extend generally from a point adjacent to the bottom end portion 58 to the top end portion 60. The first and second recesses 55a, 55b may be configured to slidably receive an actuatable end cover 56 (see, for example, FIGS. 1 and 2). Generally, the actuatable end cover 56 may be a movable cover that can be adjusted vertically to lock or unlock the vertical support's 22 position relative to the horizontal slide member 12. As will be described in more detail herein, the actuatable end cover 56 may be vertically displaced to selectively release and secure a locking mechanism between the vertical support 22 and the horizontal slide member 12 to allow the vertical support 22 to be axially displaced and secured relative to the horizontal slide member 12.

A first or left generally C-shaped bracket 57a may extend between the proximal face 50a and the distal face 50b interconnecting the left arms 51a, 51b adjacent the bottom end region 58 of the vertical support 22. Similarly, a second or right generally C-shaped bracket 57a may extend between the proximal face 50a and the distal face 50b interconnecting the right arms 53a, 53b adjacent the bottom end region 58 of the vertical support 22. Collectively, the proximal face 50a, the distal face 50b, the U-shaped bracket 54, the left C-shaped bracket 57a, and the right C-shaped bracket 57b may define a first or left axially extending channel 62a and a second or right axially extending channel 62b.

The left and right axially extending channels 62a, 62b may be sized and shaped to slide over the left and right rails 14a, 14b. Each of the axially extending channels 62a, 62b may extend from a front or proximal side 66 to a back or distal side 68 of the vertical support 22. Further each of the axially extending channels 62a, 62b may include a radially inwardly extending protrusion 64a, 64b. The radially inwardly extending protrusion 64a, 64b may have length that is less than a length of the axially extending channel 62a, 62b and less than the length of the first or second recesses 24a, 24b. For example, the radially inwardly extending protrusions 64a, 64b may be sized and shaped to be selectively received within the first or second recesses 24a, 24b. When the radially inwardly extending protrusions 64a, 64b are received within the first recesses 24a or the second recesses 24b, axial movement of the vertical support 22 may be prevented. For example, the radially inwardly extending protrusions 64a, 64b may form a mechanical stop against the edges of the recess in which it is disposed.

The vertical support 22 may further include a first laterally inwardly extending wall 61a and a second laterally inwardly extending 61b. The laterally inwardly extending walls 61a, 61b may extend into a channel 63 defined by the proximal face 50a, the distal face 50b, and the U-shaped bracket 54. The laterally inwardly extending walls 61a, 61b may be axially offset within the vertical support 22 such that the laterally inwardly extending walls 61a, 61b are positioned closer to the distal face 50b than the proximal face 50a. However, this is not required. As will be described in more detail herein, a vertical slide member 92 may be coupled relative to and slide along the laterally inwardly extending walls 61a, 61b.

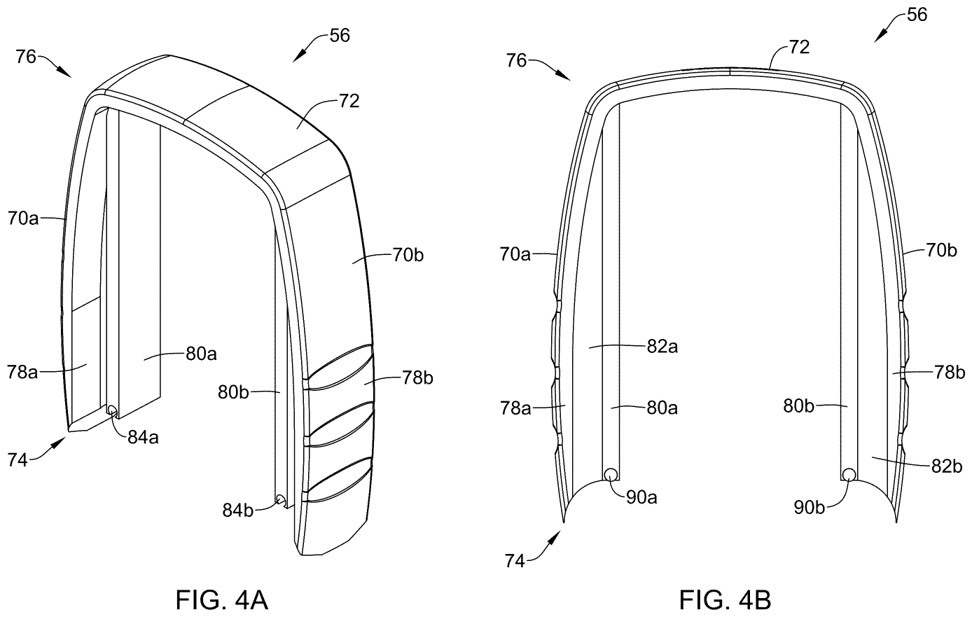

Referring additionally to FIG. 4A which is a perspective view of an illustrative actuatable end cover 56 and FIG. 4B which is a proximal view of the illustrative actuatable end cover 56, the actuatable end cover 56 may have a generally inverted “U” shape having a first or left arm 70a, a second or right arm 70b laterally spaced from the left arm 70a, and an interconnecting arm 72 extending between the left and right arms 70a, 70b. The actuatable end cover 56 may be formed as a single monolithic structure or as two or more components coupled together. The actuatable end cover 56 may extend from a bottom end region 74 to a top end region 76. The left and right arms 70a, 70b may extend generally parallel to one another. The interconnecting arm 72 may extend between left and right arms 70a, 70b adjacent the top end region 76 of the actuatable end cover 56. The interconnecting arm 72 may extend generally orthogonal to the left and right arms 70a, 70b. In some examples, the left and right arms 70a, 70b may each have a generally “I” beam shape. For example, the left arm 70a may include a laterally outward flange 78a, a laterally inward flange 80a, and an interconnecting member 82a extending between the laterally outward flange 78a and the laterally inward flange 80a. Similarly, the right arm 70b may include a laterally outward flange 78b, a laterally inward flange 80b, and an interconnecting member 82b extending between the laterally outward flange 78b and the laterally inward flange 80b. The laterally inward flanges 80a, 80b may have an axial length that is less than an axial length of the laterally outward flanges 78a, 78b. The laterally inwards flanges 80a, 80b may be sized and shaped to mate with the first and second recesses 55a, 55b of the vertical support 22. The interconnecting members 82a, 82b may have a lateral width that varies along a height of the actuatable end cover 56 such that the laterally outward flanges 78a, 78b are disposed along a laterally outward surface of the left and right arms 51a, 51b, 53a, 53b of the vertical support 22. The laterally inward flanges 80a, 80b may include a recess or channel 84a, 84b configured to receive an extension 90a, 90b (not shown in FIG. 4A) therein. The extensions 90a, 90b may extend axially from the front of the laterally inward flanges 80a, 80b and axially from the back of the laterally inward flanges 80a, 80b. The extensions 90a, 90b may be configured to engage vertical channels 86a-d formed in the proximal face 50a and the distal face 50b of the vertical support 22 to limit vertical movement of the actuatable end cover 56 relative to the vertical support 22. In some cases, the extensions 90a, 90b may be separate components that are assembled into the channels 84a, 84b after the actuatable end cover 56 is assembled with the vertical support 22. However, this is not required.

Returning to FIGS. 3A and 3B, the proximal face 50a may include a left vertical channel 86a formed in the left arm 51a thereof and a right vertical channel 86b formed in the right arm 53a thereof. Similarly, the distal face 50b may include a left vertical channel 86c formed in the left arm 51b thereof and a right vertical channel 86d formed in the right arm 53b thereof. The vertical channels 86a-d may each extend from a first end connected to the axially extending channel 62a, 62b to a second end 88a-d positioned between the bottom end region 58 and the top end region 60 of the vertical support 22. When the actuatable end cover 56 is assembled with the vertical support 22, the extensions 90a, 90b may be received within the vertical channels 86a-d. As the actuatable end cover 56 is moved up relative to the vertical support 22, the extensions 90a, 90b may contact the second end 88a-d of the vertical channels 86a-d to provide a mechanical stop between the actuatable end cover 56 and the vertical support 22.

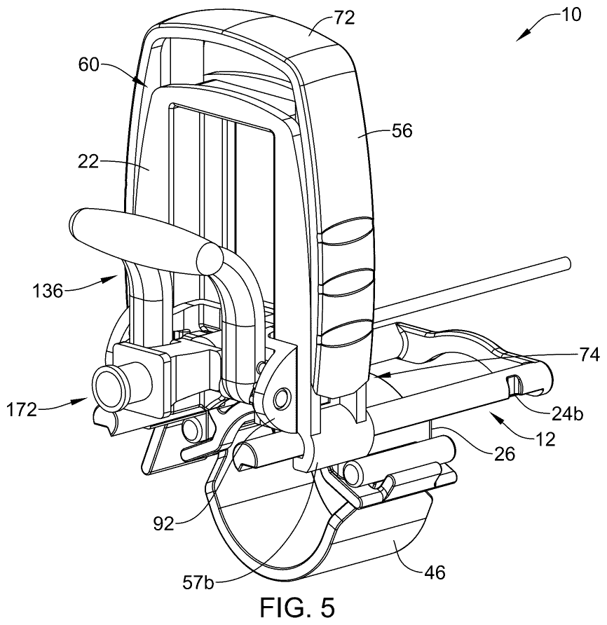

The actuatable end cover 56 may be vertically displaced within the first and second recesses 55a, 55b of the vertical support 22 to selectively bias the radially inwardly extending protrusions 64a, 64b laterally inwards or to remove a biasing force from the C-shaped brackets 57a, 57b or from the left and right arms 51a, 51b, 53a, 53b of the vertical support 22. FIG. 2 illustrates the vertical support 22 engaged with the recesses 24a closest to the proximal region 16 of the horizontal slide member 12. To axially displace the vertical support 22 along the horizontal slide member 12, the user may slide or move the actuatable end cover 56 up, as shown in FIG. 5, which is a perspective view of the guide system 10 with the actuatable end cover 56 in a second or unlocked configuration. In the second or unlocked configuration, the actuatable end cover 56 has been moved up or raised relative to the vertical support 22. For example, in the unlocked configuration, the interconnecting arm 72 of the actuatable end cover 56 is vertically spaced from the top end region 60 of the vertical support 22 and/or the interconnecting arm 52a, 52b of the proximal and distal faces 50a, 50b. Further, the bottom end region 74 of the actuatable end cover 56 is vertically spaced from the C-shaped brackets 57a, 57b and/or the bottom end region 58 of the vertical support 22. When the bottom end region 74 of the actuatable end cover 56 is vertically spaced from the C-shaped brackets 57a, 57b and/or the bottom end region 58 of the vertical support 22, the C-shaped brackets 57a, 57b may move laterally outwards to disengage the radially inward extending protrusions 64a, 64b from the recesses 24a in the left and right rails 14a, 14b. The vertical support 22 may then be axially displaced along the horizontal slide member 12. For example, the vertical support 22 may be moved towards the distal region 18 of the horizontal slide member 12. When the vertical support 22 is in the desired location, the actuatable end cover 56 may be vertically lowered to a first or locked configuration (see, for example, FIG. 1) position where the bottom end region 74 of the actuatable end cover 56 engages the C-shaped brackets 57a, 57b. When the bottom end region 74 of the actuatable end cover 56 engages the C-shaped brackets 57a, 57b, the actuatable end cover 56 may exert a biasing force on the C-shaped bracket 57a, 57b and/or the left and right arms 51a, 51b, 53a, 53b of the vertical support 22 to bias the radially inward extending protrusions 64a, 64b laterally inwards and into the recesses 24b. This may secure the vertical support 22 in place along the horizontal slide member 12.

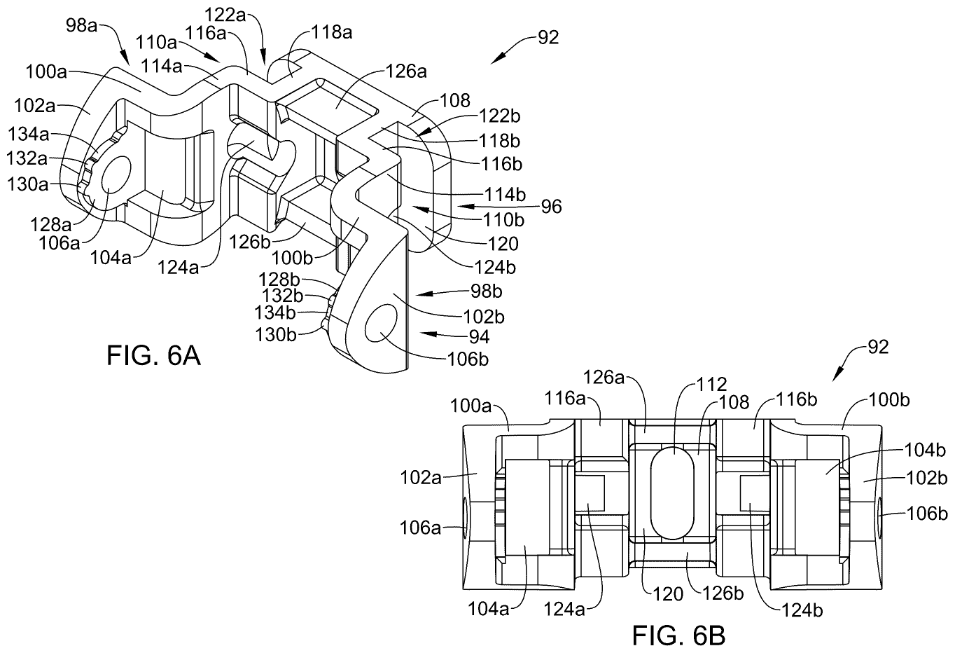

FIG. 6A is a perspective view of the illustrative vertical slide member 92 and FIG. 6B is a front view of the illustrative vertical slide member 92. The vertical slide member 92 may be formed as a single monolithic structure or as two or more components coupled together. The vertical slide member 92 may include a first or proximal region 94 configured to be coupled to the locking mechanism 136 and a second or distal region 96 configured to be movably coupled to the vertical support 22. The proximal region 94 may have a lateral width greater than a lateral width of the distal region 96. Generally, the proximal region 94 may be positioned in front of the proximal face 50a of the vertical support 22 while the distal region 96 may be positioned within the channel 63. The proximal region 94 may include a first or left bent or “L” shaped bracket 98a and a second or right bent or “L” shaped bracket 98b. Each bracket 98a may include a first member 100a, 100b and a second member 102a, 102b. The second member 102a, 102b may extend generally orthogonal to the first member 100a, 100b. A first opening 104a, 104b may extend through a thickness of each of the first member 100a, 100b. Each first opening 104a, 104b may be sized and shaped to receive one or more swivel lock cam members 142a, 142b of the locking mechanism 136 therethrough, as will be described in more detail herein. A second opening or aperture 106a, 106b may extend through a thickness of each of the second members 102a, 102b. The second apertures 106a, 106b may be sized and shaped to receive mating mounting members 148a, 148b of the locking mechanism 136. In some embodiments, the second apertures 106a, 106b may have a generally circular cross-sectional shape to allow the mounting members 148a, 148b to rotate therein. However, this is not required. The second apertures 106a, 106b may extend generally parallel to axis 4.

In some embodiments, the second members 102a, 102b may include laterally inwardly extending protrusions 128a, 128b extending around the second apertures 106a, 106b. The laterally inwardly extending protrusions 128a, 128b may each include a first protrusion 130a, 130b and a second protrusion 132a, 132b extending from a perimeter 134a, 134b thereof. The first and second protrusions 130a, 130b, 132a, 132b may be configured to engage guide members 150a, 150b of the locking mechanism 136 to provide tactile feedback to the user when the locking mechanism 136 is in a particular configuration. Further, the first and second protrusions 130a, 130b, 132a, 132b may secure the locking mechanism 136 in a desired configuration, as will be described in more detail herein.

The distal region 96 may include a distal plate 108, a first or left intermediate portion 110a, and a second or right intermediate portion 110b. The distal plate 108 may have a generally rectangular prism shape with curved corners. However, the distal plate 108 may take other shapes, as desired. The distal plate 108 may include an elongated central aperture 112 extending through a thickness thereof. The central aperture 112 may be elongated in the vertical direction to allow the elongate needle region 182 of the guide needle 172 to be angled upwards or downwards, as will be described in more detail herein. In some embodiments, a cross-section of the central aperture 112 may be generally stadium shaped or pill shaped. For example, the cross-section of the elongated central aperture 112 may be rectangular with semi-circles at a pair of opposing ends. However, other cross-sectional shapes may be used as desired, such as, but not limited to, oblong, rectangular, or the like.

The intermediate regions 110a, 110b may each include a first leg 114a, 114b, a second leg 116a, 116b, and a third leg 118a, 118b. The first legs 114a, 114b may be connected to the first members 100a, 100b of the left and right “L” brackets 98a, 98b. The third legs 118a, 118b may be connected to the distal plate 108 at locations between the central aperture 112 and the lateral (left and right) ends of the distal plate 108. The first legs 114a, 114b may extend generally parallel to each other and to the third legs 118a, 118b. The second legs 116a, 116b may extend generally orthogonal to and between the respective first legs 114a, 114b and third legs 118a, 118b. The left and right “L” brackets 98a, 98b and the intermediate regions 110a, 110b may have a generally stair-step profile when viewed from the top such that a lateral width of the vertical slide member 92 decreases in an incremental or stair-step manner from the proximal end to the distal end of the intermediate regions 110a, 110b. The distal plate 108 may have a width that is greater than a width between the third legs 118a, 118b. The second legs 116a, 116b may extend generally parallel to the distal plate 108. The second legs 116a, 116b may be spaced a distance (e.g., approximately equal to an axial length of the third legs 118a, 118b) from the front surface 120 of the distal plate 108 to define a first vertically extending slot 122a and a second vertically extending slot 122b. When the vertical slide member 92 is assembled with the vertical support 22, the slots 122a, 122b may be configured to receive the laterally inwardly extending walls 61a, 61b therein. The vertical slide member 92 may be configured to move vertically along the laterally inwardly extending walls 61a, 61b in an up and down (top to bottom) manner (e.g., parallel to axis 6).

The intermediate regions 110a, 110b may each include an aperture 124a, 124b formed therein. The apertures 124a, 124b may extend in a direction generally parallel to the lateral axis 4 and may extend through portions of the first leg 114a, 114b, portions of the second leg 116a, 116b, and portions of the third leg 118a, 118b. The apertures 124a, 124b may be sized and shaped to receive mating mounting members 148a, 148b of the needle mount 158. In some embodiments, the apertures 124a, 124b may have a generally circular cross-sectional shape to allow the mounting members 148a, 148b to rotate therein. However, this is not required.

In some embodiments, the vertical slide member 92 may further include a first wall 126a extending between the third legs 118a, 118b and a second wall 126b extending between the third legs 118a, 118b. The first wall 126a may be positioned above the central aperture 112 and the second wall 126b may be positioned below the central aperture 112. The first and second wall 126a, 126b may extend generally parallel to one another. The first and second walls 126a, 126b may provide support to the distal region 96 of the vertical slide member 92.

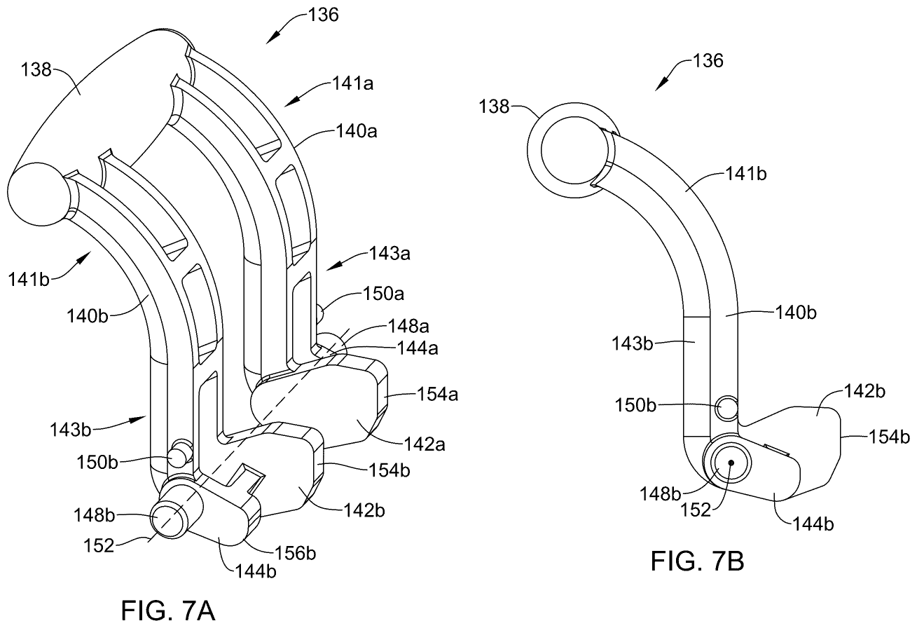

FIG. 7A is a perspective view of an illustrative actuatable locking mechanism 136. FIG. 7B is a right-side view of the illustrative actuatable locking mechanism 136. The locking mechanism 136 may generally include a gripping member or handle 138, a first or left connection member 140a extending between the handle 138 and a first swivel lock cam member 142a and a first vertical lock cam member 144a, and a right connection member 140b extending between the handle 138 and a second swivel lock cam member 142b and a second vertical lock cam member 144b. Generally, the swivel lock cam members 142a, 142b and the vertical lock cam members 144a, 144b function like rotating levers that, when turned, either engage or disengage with other parts to prevent or allow movement, as will be described in more detail herein.

The locking mechanism 136 may be formed as a single monolithic structure or may be formed from two or more components subsequently connected together. The left and right connection members 140a, 140b may each include a generally curved portion 141a, 141b and a generally linear portion 143a, 143b. The curved portion 141a, 141b may be connected to and extend from the handle 138 and the linear portion 143a, 143b may be connected and extend from the swivel lock cam member 142a, 142b and the vertical lock cam member 144a, 144b. The curved portion 141a, 141b and the linear portion 143a, 143b may intersect at an intermediate region of the connection members 140a, 140b.

A left mounting member 148a may extend laterally outward from an outer surface of the first vertical lock cam member 144a. The left mounting member 148a may be configured to be received in the left aperture 106a of the vertical slide member 92. A right mounting member 148b may extend laterally outward from an outer surface of the second vertical lock cam member 144b. The right mounting member 148b may be configured to be received in the right aperture 106b of the vertical slide member 92. The mounting members 148a, 148b may have a generally circular cross-section such that the mounting members 148a, 148b may rotate within the apertures 106a, 106b. However, other cross-sectional shapes may be used, as desired. A left guide member 150a may extend laterally outward from an outer surface of the left connection member 140a adjacent to the left mounting member 148a. For example, the left guide member 150a may be positioned near the left mounting member 148a. A right guide member 150b may extend laterally outward from an outer surface of the right connection member 140b adjacent to the right mounting member 148b. For example, the right guide member 150b may be positioned near the right mounting member 148b. The left and right guide members 150a, 150b may be configured to move along the perimeter 134a, 134b of the laterally inwardly extending protrusions 128a, 128b of the vertical slide member 92 and selectively engage the first and second protrusions 130a, 130b, 132a, 132b.

The first and second swivel lock cam members 142a, 142b may be have an irregular shape such that as the locking mechanism 136 is rotated about an axis 152 extending through a center of the left and right mounting members 148a, 148b, a distal edge 154a, 154b of the first and second swivel lock cam members 142a, 142b is configured to selectively contact and apply a biasing force to the mounting members 148a, 148b to the needle mount 158, as will be described in more detail herein. When the handle 138 is actuated towards the horizontal slide member 12, the distal edges 154a, 154b of the first and second swivel lock cam members 142a, 142b disengage from the mounting members 148a, 148b of the needle mount 158.

The first and second vertical lock cam members 144a, 144b may have a generally oblong shape such that as the locking mechanism 136 is rotated about an axis 152 extending through a center of the left and right mounting members 148a, 148b, a curved edge 156b of the first and second vertical lock cam members 144a, 144b is configured to selectively contact and apply a biasing force to the proximal face 50a of the vertical support 22, as will be described in more detail herein. When the handle 138 is actuated towards the horizontal slide member 12, the curved edges 156b of the first and second vertical lock cam members 144a, 144b disengage from the proximal face 50a of the vertical support 22.

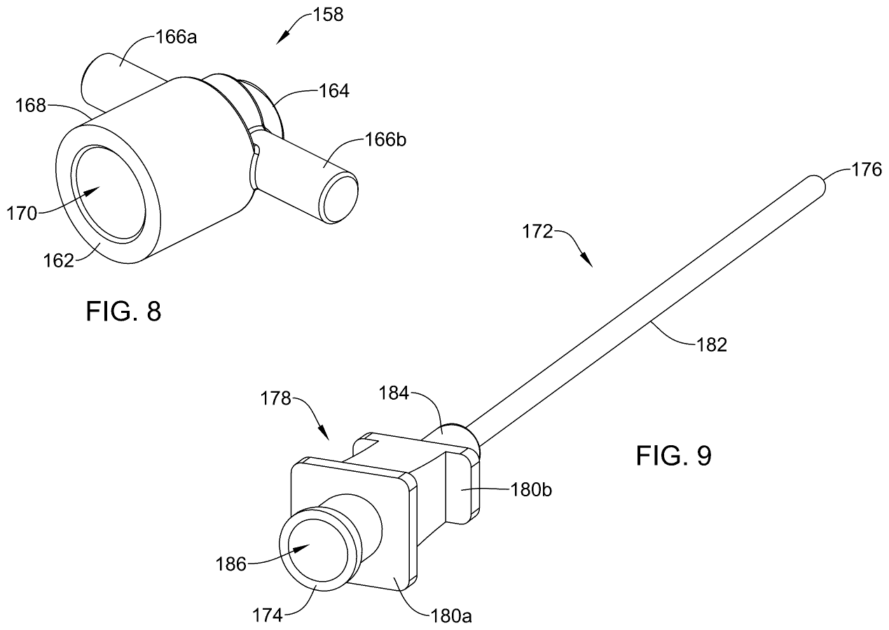

FIG. 8 is a perspective view of an illustrative needle mount 158. The needle mount 158 may include a generally tubular body 168 extending from a proximal end 162 to a distal end 164. A first or left mounting member 166a may extend laterally outward from an outer surface of the tubular body 168 and a second or right mounting member 166b may extend laterally outward from an opposing outer surface of the tubular body 168. The left member 166a may be configured to be received in the left aperture 124a of the left intermediate region 110a of the vertical slide member 92. The right mounting member 166b may be configured to be received in the right aperture 124b of the right intermediate region 110b of the vertical slide member 92. The mounting members 166a, 166b may have a generally circular cross-section such that the mounting members 166a, 166b may rotate within the apertures 124a, 124b.

The tubular body 168 may define a lumen 170 for receiving a portion of the guide needle 172 therethrough. An inner diameter of the tubular body 168 may reduce between the proximal end 162 and the distal end 164. In some cases, the inner diameter may reduce in a tapered or gradual manner. In other cases, the inner diameter may reduce in an abrupt or stair-step manner. In some examples, the inner diameter of the tubular body 168 adjacent to the proximal end 162 may be configured to form a friction fit with a transition region 184 of the guide needle 172 to releasably secure the guide needle 172 within the needle mount 158. However, this is not required.

FIG. 9 is a perspective view of an illustrative guide needle 172. The guide needle 172 may extend from a proximal end 174 to distal end 176. The distal end 176 may be configured to be inserted into the patient. The guide needle 172 may include a gripping region 178 adjacent to the proximal end 174 thereof. The gripping region 178 may include flanges 180a, 180b extending laterally therefrom to facilitate gripping of the guide needle 172. An elongate needle region 182 may have smaller profile than the gripping region 178 to facilitate insertion into the body. A transition region 184 may be positioned between the gripping region 178 and the elongate needle region 182. As described above, an outer surface of the transition region 184 may form a friction fit with the inner surface of the tubular body of the needle mount 158 to releasably secure the guide needle 172 within the needle mount 158. A lumen 186 may extend from the proximal end 174 to the distal end 176 of the guide needle 172. The lumen 186 may be configured to receive a biopsy needle (not explicitly shown) therethrough. A diameter of the lumen 186 may reduce between the proximal end 174 and the distal end 176. In some cases, the diameter may reduce in a tapered or gradual manner. In other cases, the diameter may reduce in an abrupt or stair-step manner.

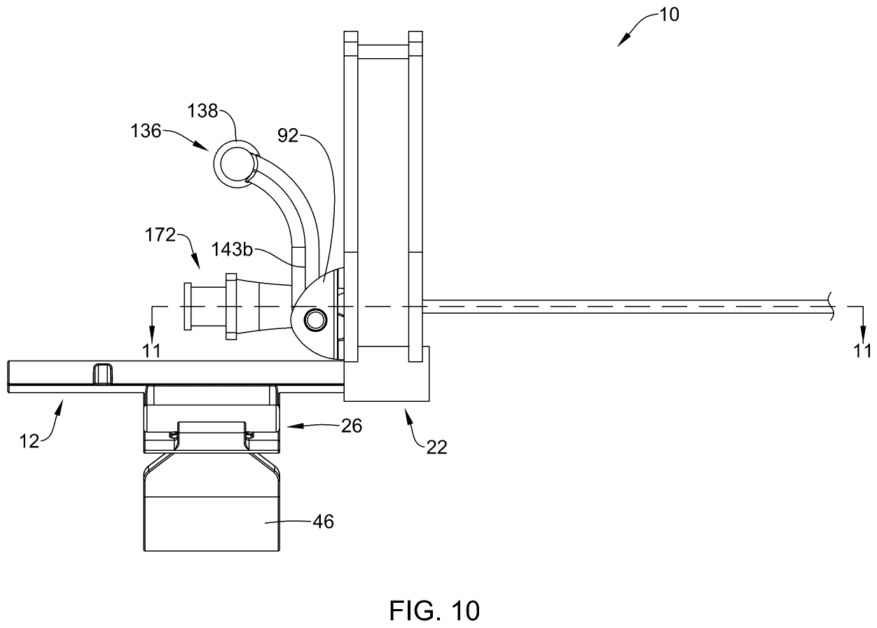

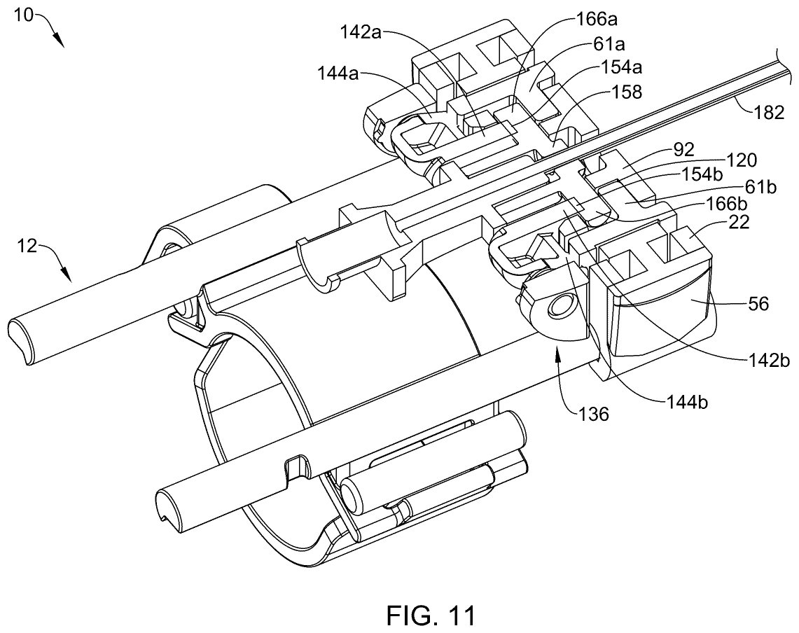

The locking mechanism 136 may be movable between a locked first configuration (shown in FIGS. 1, 2, and 5) in which both vertical movement of the vertical slide member 92 and swivel (up and down angulation) movement of the needle mount 158 are prevented, a partially locked second configuration in which vertical movement of the vertical slide member 92 is prevented while allowing swivel movement of the needle mount 158, and an unlocked third configuration in which both vertical movement of the vertical slide member 92 and swivel movement needle mount 158 are allowed. FIG. 10 is a partial side view of the guide system 10 with the locking mechanism 136 in the first configuration. FIG. 11 is a partial cross-sectional view of the guide system 10, taken at line 11-11 of FIG. 10. When the locking mechanism 136 is in the first configuration, the generally linear portions 143a, 143b of the connection members 140a, 140b may extend in the vertical direction (e.g., generally parallel to axis 6). In the first configuration, the distal edges 154a, 154b of the swivel lock cam members 142a, 142b contact and push against the mounting members 166a, 166b of the needle mount 158. The engagement of the swivel lock cam members 142a, 142b with the mounting members 166a, 166b of the needle mount 158 may prevent the mounting members 166a, 166b from rotating or pivoting within the apertures 124a, 124b and thus prevent swivel (angular) movement of the needle mount 158. For example, the mechanical force of the swivel lock cam members 142a, 142b with the mounting members 166a, 166b may create a frictional and/or mechanical engagement therebetween to prevent rotation of the needle mount 158. In the first configuration, the vertical lock cam members 144a, 144b may extend through the openings 104a, 104b to contact and push against the proximal face 50a of the vertical support 22 at the left and right arms 51a, 53a thereof. The vertical lock cam members 144a, 144b may exert a force against the proximal face 50a which axially displaces the vertical support 22 such that a distal surface of the laterally inwardly extending walls 61a, 61b is pushed into and contacts the front surface 120 of the distal plate 108 of the vertical slide member 92. The frictional and/or mechanical engagement between the laterally inwardly extending walls 61a, 61b and the front surface 120 of the vertical slide member 92 may prevent the vertical slide member 92 from sliding vertically up and down along the laterally inwardly extending walls 61a, 61b.

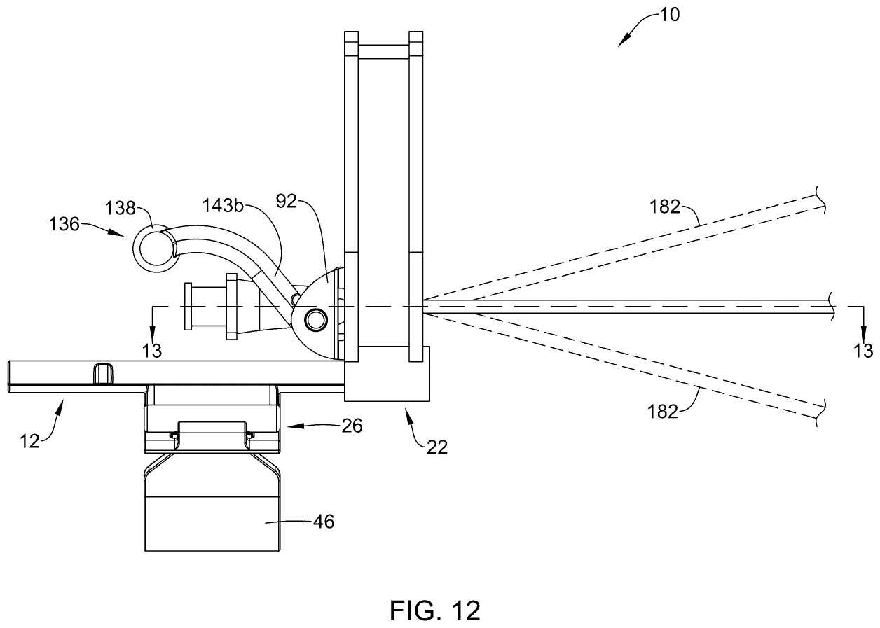

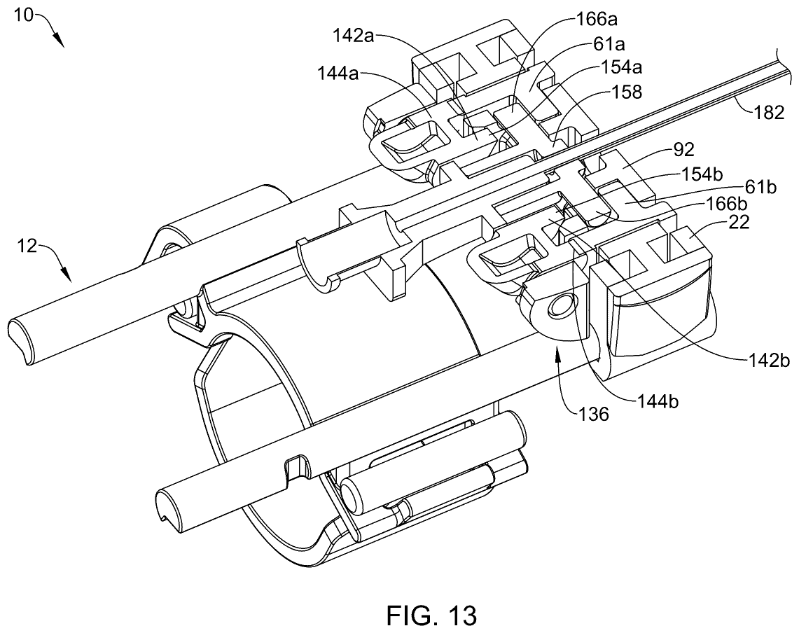

FIG. 12 is a partial side view of the guide system 10 with the locking mechanism 136 in the second, partially locked, configuration. FIG. 13 is a partial cross-sectional view of the guide system 10, taken at line 13-13 of FIG. 12. When the locking mechanism 136 is in the second configuration, the handle 138 is moved towards the horizontal slide member 12 such that the generally linear portions 143a, 143b of the connection members 140a, 140b may extend in a non-vertical direction (e.g., generally non-parallel to axis 6). In some examples, the linear portions 143a, 143b may extend at an angle in the range of about 25° to about 45° relative to the vertical axis 6. However, the angle may be less than 25° or greater than 45°, as desired. In the second configuration, the distal edges 154a, 154b of the swivel lock cam members 142a, 142b are spaced a distance from the mounting members 166a, 166b of the needle mount 158. This may allow the mounting members 166a, 166b to rotate within the apertures 124a, 124b to allow the elongate needle region 182 to be angled relative to the horizontal axis 2. For example, the needle mount 158 may be rotated within the apertures 124a, 124b to allow the elongated needle region 182 of the guide needle 172 to extend at an angle parallel to or non-parallel to the horizontal axis 2. For example, FIG. 12 illustrates some alternative positions of the elongated needle region 182 of the guide needle 172. While not explicitly shown the proximal end region of the guide needle 172 angles with elongated needle region 182. It is contemplated that the extent to which the guide needle 172 may be offset (up or down) from parallel to the horizontal axis 2 may be determined at least in part by a height of the elongated central aperture 112. For example, upward angulation may be limited by the upper edge of the elongated central aperture 112 and downward angulation may be limited by the lower edge of the elongated central aperture 112. In the second configuration, the vertical lock cam members 144a, 144b may extend through the openings 104a, 104b to contact and push against the proximal face 50a of the vertical support 22 at the left and right arms 51a, 53a thereof. The vertical lock cam members 144a, 144b may exert a force against the proximal face 50a which axially displaces the vertical support 22 such that a distal surface of the laterally inwardly extending walls 61a, 61b is pushed into and contacts the front surface 120 of the distal plate 108 of the vertical slide member 92. The frictional and/or mechanical engagement between the laterally inwardly extending walls 61a, 61b and the front surface 120 of the vertical slide member 92 may prevent the vertical slide member 92 from sliding vertically up and down along the laterally inwardly extending walls 61a, 61b.

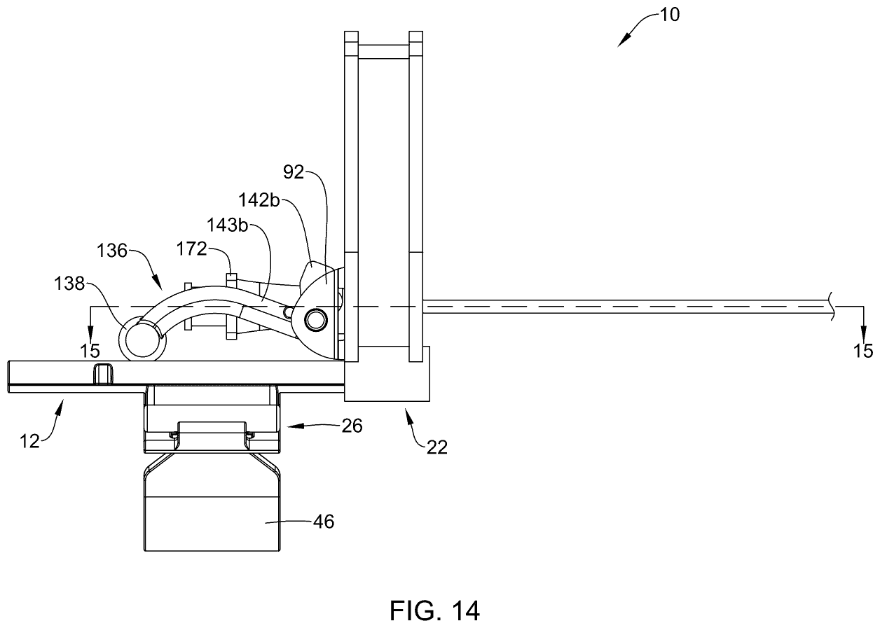

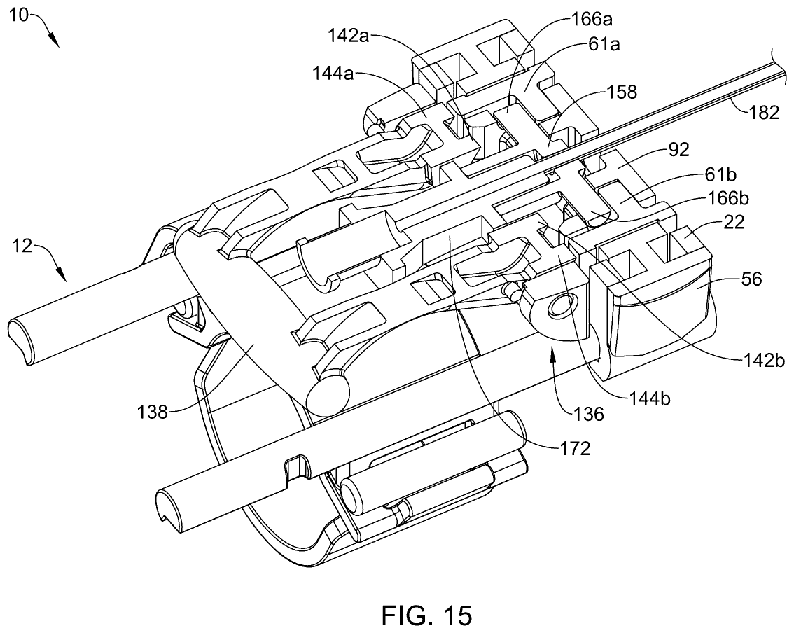

FIG. 14 is a partial side view of the guide system 10 with the locking mechanism 136 in the third, fully unlocked, configuration. FIG. 15 is a partial cross-sectional view of the guide system 10, taken at line 15-15 of FIG. 14. When the locking mechanism 136 is in the third configuration, the handle 138 is moved towards the horizontal guide member 12 such that the generally linear portions 143a, 143b of the connection members 140a, 140b may extend in a non-vertical direction (e.g., generally non-parallel to axis 6). In some examples, the linear portions 143a, 143b may extend at an angle in the range of about greater than 50° relative to the vertical axis 6. However, the angle may be less than 50°, but greater than the angle of the second configuration, as desired. In the third configuration, the distal edges 154a, 154b of the swivel lock cam members 142a, 142b are spaced a distance from the mounting members 166a, 166b of the needle mount 158. This may allow the mounting members 166a, 166b to rotate within the apertures 124a, 124b to allow the elongate needle region 182 to be angled relative to the horizontal axis 2. For example, the needle mount 158 may be rotated within the apertures 124a, 124b to allow the elongate needle region 182 of the guide needle 172 to extend at an angle parallel or non-parallel to the horizontal axis 2. It is contemplated that the extent to which the guide needle 172 may be offset (up or down) from the horizontal axis 2 may be determined at least in part by a height of the elongated central aperture 112. For example, upward angulation may be limited by the upper edge of the elongated central aperture 112 and downward angulation may be limited by the lower edge of the elongated central aperture 112. In the third configuration, the vertical lock cam members 144a, 144b may be spaced from the proximal face 50a of the vertical support 22 at the left and right arms 51a, 53a thereof. This may remove the frictional and/or mechanical engagement between the distal surface of the laterally inwardly extending walls 61a, 61b and the front surface 120 of the distal plate 108 of the vertical slide member 92 allowing the vertical slide member 92 to move vertically along the laterally inwardly extending walls 61a, 61b in an up and down (top to bottom) direction. A laterally extending surface 188 of the vertical support 22 may limit upwards movement of the vertical slide member 92 and the rails 14a, 14b may limit downwards movement of the vertical slide member 92.

In use, the guide system 10 may be secured to an ultrasound probe using the strap 46 before or after the ultrasound probe is positioned within the anatomy. For example, for a prostate biopsy, the ultrasound probe may be positioned within the rectum. Once the guide system 10 is secured to the ultrasound probe and the ultrasound probe is positioned, the vertical support 22 may be axially displaced along the horizontal slide member 12 and secured in the desired axial position. This may be performed with the locking mechanism 136 in the first (locked), second (partially locked), or third (unlocked) configurations. Next, a vertical position of the vertical slide member 92 (and thus a vertical position of the guide needle 172) may be adjusted. The locking mechanism 136 may be moved to the third or unlocked configuration to allow the vertical slide member 92 to be moved along the laterally inwardly extending walls 61a, 61b of the vertical support 22. Once the vertical slide member 92 is at the desired height or vertical location, the locking mechanism 136 may be moved to the second or partially locked configuration to lock the vertical position of the vertical slide member 92. As the locking mechanism 136 is moved from the third or fully unlocked configuration to the second or partially locked configuration, the guide members 150a, 150b of the locking mechanism 136 may be advanced over the first protrusions 130a, 130b on the vertical slide member 92. This may provide some resistance that indicates to the user the locking mechanism 136 has been moved to a different functional position. Similarly, the second protrusions 132a, 132b may provide a mechanical stop indication to the user to indicate when the locking mechanism 136 is being moved to the first or locked configuration. When the locking mechanism 136 is in the second or partially locked configuration, the guide members 150a, 150b may be positioned between the first protrusions 130a, 130b and the second protrusions 132a, 132b. In the second or partially locked configuration, the angle of the needle mount 158 may be adjusted to position the guide needle 172 at a desired angle. Once the needle mount 158 is in the desired position, the locking mechanism 136 may be moved to the first or locked configuration to lock or secure the needle mount 158 at the desired angle. As the locking mechanism 136 is moved from the second or partially locked configuration to the first or locked configuration, the guide members 150a, 150b of the locking mechanism 136 may be advanced over the second protrusions 132a, 132b. This may provide some resistance that indicates to the user the locking mechanism 136 has been moved to a different functional position. As the first protrusions 130a, 130b and the second protrusions 132a, 132b provide some resistance to movement of the locking mechanism 136, the protrusions 130a, 130b, 132a, 132b may help hold the locking mechanism 136 in the desired configuration and limit unintended actuation thereof. It is contemplated that the locking mechanism 136 and/or other portions of the guide system 10 may include visual markings to indicate to the user when the locking mechanism 136 is in a fully locked, a partially locked, or fully unlocked configuration.

It is contemplated that multiple samples may be taken with the vertical slide member 92 at a same height. For example, the angle of the guide needle 172 may be adjusted to obtain multiple samples within a same plane. It is further contemplated that the order of the steps described herein may be rearranged. For example, the vertical and/or angular position of the guide needle 172 may be adjusted prior to moving the vertical support 22 along the horizontal slide member 12 towards or away from the patient's body.

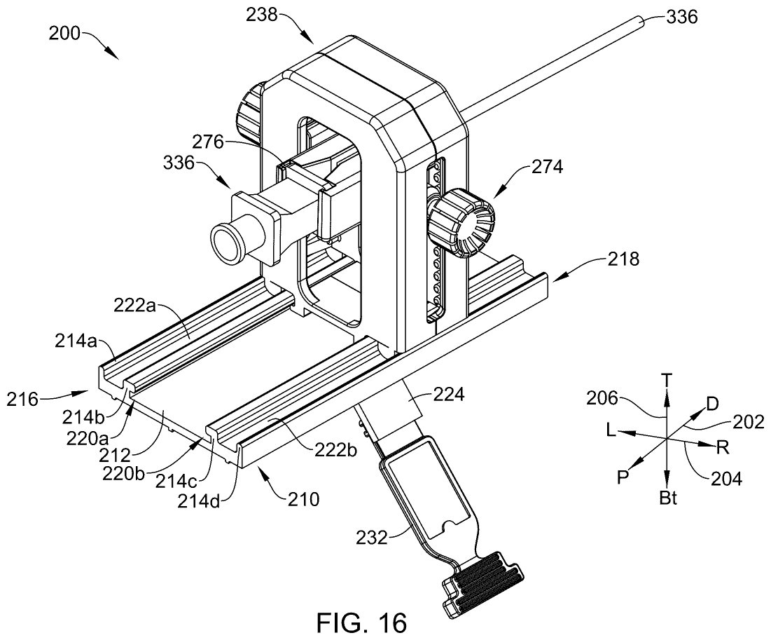

FIG. 16 is a perspective view another illustrative prostate biopsy guide system 200 in a first configuration. While the guide system 200 is described with respect to prostate biopsy, it is contemplated that the guide system 200 may be used in other procedures. In one illustrative example, the guide system 200 may be used to facilitate the injection of a spacing material, such as between the rectum and the prostate to prevent damage during radiation therapy. One illustrative system for injecting materials into the space between the rectum and the prostate is the SpaceOAR Vue™ System available from Boston Scientific Corporation, Marlborough, MA, USA. The guide system 200 may be configured to allow for horizontal, vertical, and angular movement of the biopsy needle (not explicitly shown) while also fixing the biopsy needle in a particular configuration. The guide system 200 may be configured to mount to an ultrasound probe (not explicitly shown) such that the guide system 200 is in a generally fixed position relative to the ultrasound probe. As used herein, movement in the horizontal direction or relational positioning in a direction generally parallel to axis 202, will be described as axial movement or an axial direction with relational positioning using proximal (P) or front and distal (D) or back; movement or relational positioning in a direction generally parallel to axis 204 will be described as lateral movement or a lateral direction with relational positioning using left (L) and right (R); and movement or relational positioning in a direction generally parallel to axis 206 will be described as vertical movement or a vertical direction with relational positioning using top (T) or up and bottom (Bt) or down. The use of the terms “proximal”, “distal”, “front,” “back”, “left”, “right”, “top”, “bottom”, “up”, and “down” are not intended to limit the guide system 200 to a particular orientation, but rather facilitate discussion of relative orientation.

Generally, the guide system 200 may be configured to control a horizontal position, a vertical position, and/or an angle of a guide needle. The guide system 200 may be configured to lock the guide needle 330 in a desired orientation independent of an ultrasound probe. Further, the vertical and/or angular position may be held or locked as the guide needle 330 is moved towards the patient's body and/or away from the patient's body. The guide system 200 may include a horizontal slide member 210, a vertical housing 238, a needle mount member 276, and a locking and actuation mechanism 274, among other features. The needle mount member 276 may be configured to move up and down (e.g., from top to bottom) along the vertical housing 238 to control a vertical position of the needle mount member 276 (and thus the guide needle 330). The locking and actuation mechanism 274 may be configured to selectively lock a vertical position of the needle mount member 276 and an angular position of the needle mount member 276. The vertical housing 238 may be moved along the horizontal slide member 210 to move the guide needle 330 towards and/or way from the patient. The vertical housing 238 may be selectively secured to the horizontal slide member 210 to lock the vertical housing 238 in a desired axial position, although this is not required.

The horizontal slide member 210 may include a generally planar base portion 212 having a plurality of axially extending rails 214a-d extending from a proximal region 216 to a distal region 218 of the base portion 212. The rails 214a-d may extend upwards from a top surface of the generally planar base portion 212. In some cases, the laterally outward most rails 214a, 214d may be generally linear and extend generally orthogonal to the generally planar base portion 212. The laterally inwards rails 214b, 214c may have an inverted “L” shape each defining an axially extending channel 220a, 220b extending from the proximal region 216 to the distal region 218 of the horizontal slide member 210. The channels 220a, 220b may be configured to receive a mating rail 264a-d of the vertical housing 238. A first or left laterally outward rail 214a may extend along the left edge of the generally planar base portion 212. A first or left laterally inward rail 214b may be laterally spaced from the left laterally outward rail 214a to define a left axially extending recess 222a configured to receive one or more protrusions 262a, 262c extending from a lower surface of the vertical housing 238. Similarly, a second or right laterally outward rail 214d may extend along the right edge of the generally planar base portion 212. A second or right laterally inward rail 214c may be laterally spaced from the right laterally outward rail 214a to define a right axially extending recess 222b configured to receive one or more protrusions 262b, 262d extending from a lower surface of the vertical housing 238.

The left laterally inward rail 214b may be laterally spaced from the right laterally inward rail 214c. A bottom or lower portion of the laterally inward rails 214b, 214c may be spaced a greater distance from one another than a top or upper portion of the laterally inward rails 214b, 214c to define the axially extending channels 220a, 220b.

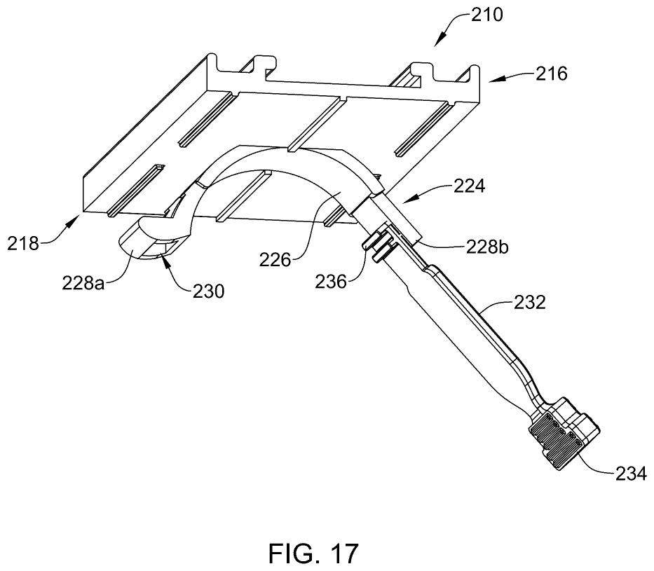

Referring additionally to FIG. 17, which is a bottom perspective view of the horizontal slide member 210, the horizontal slide member 210 may further include a probe mount 224 extending from a bottom surface of the generally planar base portion 212. The probe mount 224 may be positioned between the proximal region 216 and the distal region 218. In some embodiments, the probe mount 224 may be formed as single monolithic structure with the generally planar base portion 212. In other embodiments, the probe mount 224 may be formed as a separate component from the generally planar base portion 212 and subsequently coupled with the generally planar base portion 212. The probe mount 224 may include a concave lower or bottom surface 226 configured to conform to an outer surface of the ultrasound probe. For example, the concave lower surface 226 may rest on an outer surface of the ultrasound probe. Said differently, the probe mount 224 may include an arch or a curve extending between a first or left lateral end 228a and a second or right lateral end 228b. The probe mount 224 may be connected to the generally planar base portion 212 at a central region between the left lateral end 228a and the right lateral end 228b. One or both of the left and right lateral ends 228a, 228b of the probe mount 224 may extend laterally beyond the left and right lateral edges of the generally planar base portion 212, respectively. However, this is not required.

The probe mount 224 may include a first or left opening or aperture 230 positioned adjacent to the left lateral end 228a and extending through a thickness thereof. In some embodiments, the first opening 230 may extend through a generally linear portion of the probe mount 224 extending laterally from the curved portion thereof. The first opening 230 may be configured to receive a left end 234 of a strap 232. An opposing end 236 of the strap 232 may be fixedly or releasably secured adjacent to the right lateral end 228b of the probe mount 224. For example, the probe mount 224 may be overmolded with the right end 236 of the strap 232 or the right end of the strap 232 may form a friction fit with a mating feature on the probe mount 224. The left end 234 of the strap 232 may be sized and shaped to allow the left end 234 to be inserted at an angle through the opening 230 in a bottom to top manner. The left end 234 of the strap 232 may have a length greater than a length of the first opening 230 such that the first end 234 of the strap 232 may rest on the probe mount 224. When the left end 234 of the strap 232 is secured relative to the opening 230, the strap 232 may be configured to secure an ultrasound probe to the probe mount 224. For example, the ultrasound probe may be positioned between the concave lower surface 226 and a surface of the strap 232 to releasably secure the ultrasound probe to the guide system 200. The strap 232 may be uncoupled from the opening 230 by angling the end 234 and lowering it through the opening 230. The strap 232 may be formed from a flexible or deformable material configured to be wrapped around a lower surface of an ultrasound probe. It is contemplated that the guide system 200 may include additional or alternative securement mechanisms for securing the ultrasound probe relative to the guide system 200.

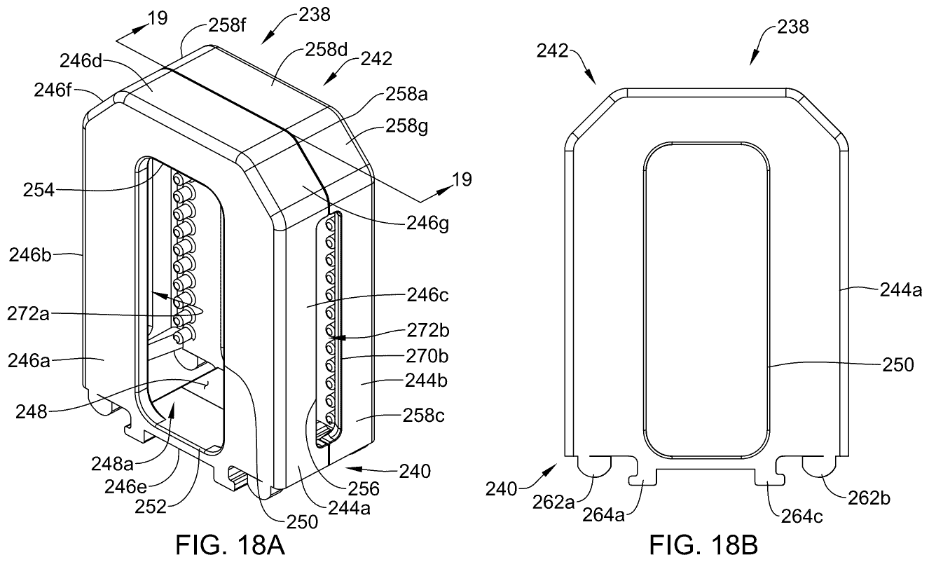

The vertical housing 238 may be configured to be axially displaced along the horizontal slide member 210. FIG. 18A is a perspective view of an illustrative vertical housing 238 and FIG. 18B is a front view of the illustrative vertical housing 238. The vertical housing 238 may extend from a bottom end region 240 to a top end region 242. The vertical housing 238 may be formed as a single monolithic structure or as two or more components coupled together. In some embodiments, the vertical housing 238 may include a proximal housing portion 244a and a distal housing portion 244b releasably coupled together. The distal housing portion 244b may be a mirror image of the proximal housing portion 244a, although this is not required. When provided as two separate components, the proximal housing portion 244a and the distal housing portion 244b may include features such as, but not limited to mating friction fits, mating snap fits, or the like, to releasably secure the two components together.

The proximal housing portion 244a may include at least a proximal wall 246a, a left lateral wall 246b, a right lateral wall 246c, a top wall 246d, and a bottom wall 246e. In some cases, the proximal housing portion 244a may further include a left angular wall 246f and a right angular wall 246g. However, this is not required. The left and right lateral walls 246b, 246c may extend between the bottom wall 246e and the left and right angular walls 246f, 246g. The bottom wall 246e extends generally orthogonal to and between the left and right lateral walls 246b, 246c adjacent to the bottom end region 240 of the vertical housing 238. The left and right angular walls 246f, 246g may extend between the top wall 246d and the top end region of the left and right lateral walls 246b, 246c. The left and right angular walls 246f, 246g may extend at a non-orthogonal angle relative to the top wall 246d and/or the left and right lateral walls 246b, 246c. In some cases, the left and right angular walls 246f, 246g may be omitted and the left and right lateral walls 246b, 246c may extend to the top wall 246d. The top wall 246d may extend generally parallel to the bottom wall 246e. The proximal wall 246a may extend between the left and right lateral walls 246b, 246c, the top and bottom walls 246e, 246e, and the left and right angular walls 246f, 246g. The back or distal region of the proximal housing portion 244a may be free from a distal wall. The walls 246a-g collectively define an interior cavity 248a. The interior cavity 248a may be configured to movably receive the needle mount member 276 therein. The proximal wall 246a may define a central opening 250 extending from a bottom end 252 to a top end 254. The height of the central opening 250 may be determined by a desired vertical travel distance of the needle mount member 276. For example, the greater the height of the central opening 250, the greater the vertical travel distance of the needle mount member 276 may be. In the illustrated embodiment, the central opening 250 has a height that is almost the same as a height of the proximal housing portion 244a. However, this is not required.

The left and right lateral walls 246b, 246c may each include a generally rectangular cut-out region 256 (not explicitly shown in the left lateral wall 246b). The cut-out region 256 may have a height that is the same as or less than the height of the central opening 250. However, this is not required. The cut-out regions 256 may each define an opening for a portion of the locking and actuation mechanism 274 to pass therethrough.

The distal housing portion 244b may include at least a distal wall 258a, a left lateral wall 258b, a right lateral wall 258c, a top wall 258d, and a bottom wall 258e. In some cases, the distal housing portion 244b may further include a left angular wall 258f and a right angular wall 258g. However, this is not required. The left and right lateral walls 258b, 258c may extend between the bottom wall 258e and the left and right angular walls 258f, 258g. The bottom wall 258e extends generally orthogonal to and between the left and right lateral walls 258b, 258c adjacent to the bottom end region 240 of the vertical housing 238. The left and right angular walls 258f, 258g may extend between the top wall 258d and the top end region of the left and right lateral walls 258b, 258c. The left and right angular walls 258f, 258g may extend at a non-orthogonal angle relative to the top wall 258d and/or the left and right lateral walls 258b, 258c. In some cases, the left and right angular walls 258f, 258g may be omitted and the left and right lateral walls 258b, 258c may extend to the top wall 258d. The top wall 258d may extend generally parallel to the bottom wall 258e. The distal wall 258a may extend between the left and right lateral walls 258b, 258c, the top and bottom walls 258e, 258e, and the left and right angular walls 258f, 258g. The front of the distal housing portion 244b may be free from a proximal wall. The walls 258a-g collectively define an interior cavity 248b. The interior cavity 248b may be configured to movably receive the needle mount member 276 therein. The distal wall 258a may define a central opening 260 extending from a bottom end 262 to a top end 264. The height of the central opening 260 may be determined by a desired vertical travel distance of the needle mount member 276. For example, the greater the height of the central opening 260, the greater the vertical travel distance of the needle mount member 276 may be. In the illustrated embodiment, the central opening 260 has a height that is almost the same as a height of the distal housing portion 244b. However, this is not required.

The left and right lateral walls 258b, 258c may each include a generally rectangular cut-out region 270a, 270b. The cut-out regions 270a, 270b may have a height that is the same as or less than the height of the central opening 260. However, this is not required. The cut-out regions 270a, 270b may each define an opening for a portion of the locking and actuation mechanism 274 to pass therethrough.

When the proximal housing portion 244a is assembled with the distal housing portion 244b, the cut-out regions 256 of the proximal housing portion 244a align with the cut-out regions 270a, 270b of the distal housing portion 244b to form a left lateral side opening 272a and a right lateral side opening 272b. Further, the central opening 250 in the proximal housing portion 244a may be aligned with the central opening 260 in the distal housing portion 244b such that an opening extends through an entire axial thickness of the vertical housing 238. Further, the interior cavities 248a, 248b may form one large cavity 248.

Each of the proximal housing portion 244a and the distal housing portion 244b may include a left protrusion 262a, 262c and a right protrusion 262b, 262d. The protrusions 262a-d may extend downward from an outer surface of the bottom wall 246e, 258e and have an axial length that is less than the axial length of the proximal housing portion 244a and/or the distal housing portion 244b. The protrusions 262a-d may be positioned near the left and right lateral edges of the vertical housing 238 and may be configured to be received within the axially extending recesses 222a, 222b of the horizontal slide member 210. In some cases, the protrusions 262a-d may be sized and shaped to slide along the length of the axially extending recesses 222a, 222b. Each of the proximal housing portion 244a and the distal housing portion 244b may further include a left axially extending rail 264a, 264b and a right axially extending rail 264c, 264d. The left axially extending rails 264a, 264b may be laterally aligned to form a continuous rail extending from the proximal end region of the vertical housing 238 to the distal end region of the vertical housing 238. Similarly, the right axially extending rails 264c, 264d may be laterally aligned to form a continuous rail extending from the proximal end region of the vertical housing 238 to the distal end region of the vertical housing 238. The axially extending rails 264a-d may have a generally “L” shape with the bottom portion thereof configured to be received within the axially extending channels 220a, 220b of the horizontal slide member 210.

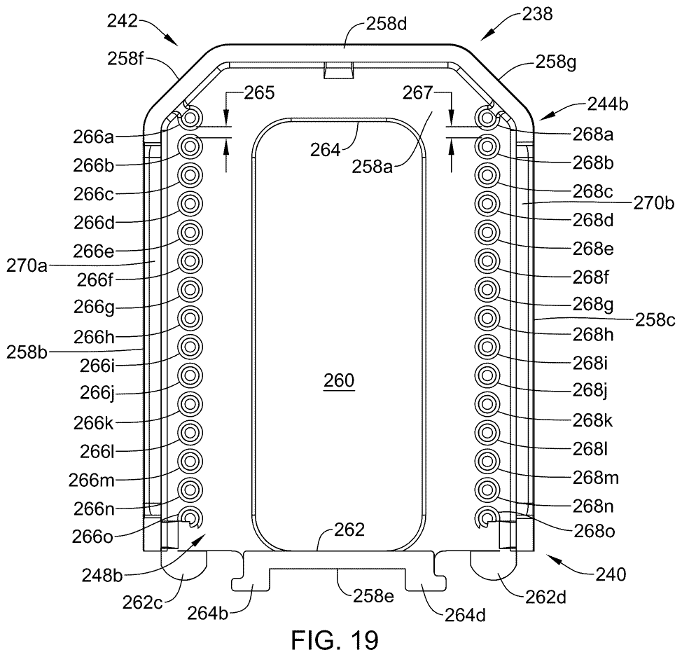

Referring additionally to FIG. 19, which is a cross-sectional view of the illustrative vertical housing 238, taken at line 19-19 of FIG. 18A, the distal housing portion 244b may further include a first plurality of dowels or pins 266a-o extending in a vertical array parallel to and adjacent to the left lateral side wall 258b. The first plurality of pins 266a-o may extend into the cavity 248b from an interior surface of the distal wall 258a. The plurality of pins 266a-o may be uniformly spaced from one another to define a gap 265 between adjacent pins. The distal housing portion 244b may further include a second plurality of dowels or pins 268a-o extending in a vertical array parallel to the right lateral side wall 258c. The second plurality of pins 268a-o may extend into the cavity 248b from an interior surface of the distal wall 258a. The second plurality of pins 268a-o may be uniformly spaced from one another to define a gap between adjacent pins. The first plurality of pins 266a-o and the second plurality of pins 268a-o may be positioned at similar vertical locations. For example, the uppermost pin 266a of the first plurality of pins 266a-o may be vertically aligned with the uppermost pin 268a of the second plurality of pins 268a-o. The spacing of the first and second plurality of pins 266a-o, 268a-o may be the same so that each pin of the first plurality of pins 266a-o is vertically aligned with the corresponding pin of the second plurality of pins 268a-o.

While not explicitly shown, the proximal housing portion 244a may similarly include first plurality of dowels or pins extending in a vertical array parallel to and adjacent to the left lateral side wall 246b. The first plurality of pins may extend into the cavity 248a from an interior surface of the proximal wall 246a. The plurality of pins may be uniformly spaced from one another to define a gap between adjacent pins. The proximal housing portion 244a may further include a second plurality of dowels or pins extending in a vertical array parallel to the right lateral side wall 246c. The second plurality of pins may extend into the cavity 248a from an interior surface of the proximal wall 246a. The plurality of pins may be uniformly spaced from one another to define a gap between adjacent pins. The first plurality of pins and the second plurality of pins may be positioned at similar vertical locations as described with respect to the first and second plurality of pins 266a-o, 268a-o of the distal housing portion 244b. The spacing of the first and second plurality of pins may be the same so that each pin of the first plurality of pins is vertically aligned with the corresponding pin of the second plurality of pins. Further, the pins of the proximal housing portion 244a may be vertically aligned with corresponding pins of the distal housing portion 244b. The proximal housing portion 244a may include a same number of pins as the distal housing portion 244b.

While the illustrated embodiment includes fifteen positioning protrusions or pins 266a-o, 268a-o in each array of pins, there may be fewer than fifteen or more than fifteen positioning protrusions or pins 266a-o, 268a-o, as desired. It is contemplated that increasing the number of pins in each array 266a-o, 268a-o may increase a number of possible vertical height locations of the needle mount member 276 while decreasing the number of pins in each array 266a-o, 268a-o may decrease a number of possible vertical height locations of the needle mount member 276. Further, while the first and second plurality of pins 266a-o, 268a-o are illustrated as having a generally cylindrical shape, the first and second plurality of pins 266a-o, 268a-o may take other shapes, as desired. Collectively, the first and second plurality of pins 266a-o, 268a-o of the distal housing portion 244b and the pins of the proximal housing portion 244a may form a rack for receiving a securement member 326a, 326b of a floating locking member 296a, 296b. When the securement member 326a, 326b is disposed between adjacent pins, vertical adjustment of the needle mount member 276 may be prevented or precluded. The securement member 326a, 326b may be selectively displaced to allow the needle mount member 276 to be moved up and down along the vertical housing 238, as will be described in more detail herein.

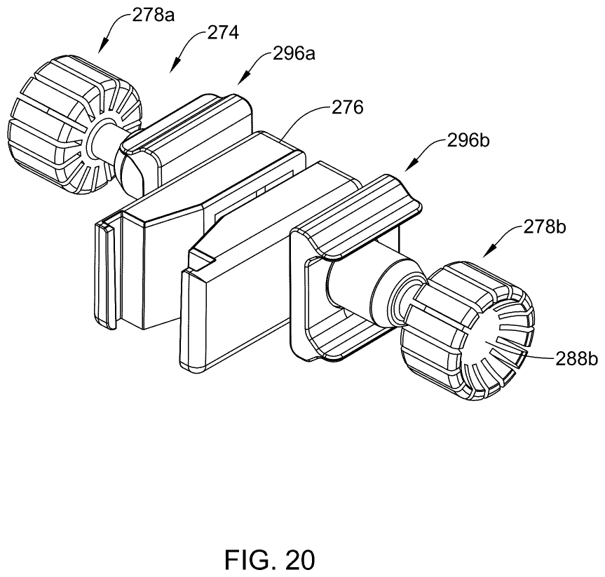

FIG. 20 is a perspective view of an illustrative locking and actuation mechanism 274 assembled with a needle mount member 276. Generally, the needle mount member 276 may be at least partially disposed within the cavity 248 of the vertical housing 238. The needle mount member 276 may be configured to move up and/or down (e.g., between the top and bottom) within the cavity 248 to position the guide needle 330 at a desired vertical location. The locking and actuation mechanism 274 may be configured to be actuated to move the needle mount member 276 vertically. In some cases, the locking and actuation mechanism 274 may also be actuated to angle the needle mount member 276. The locking and actuation mechanism 274 may include a first or left push button 278a and a second or right push button 278b. The left and right push buttons 278a, 278b may have a same structure.

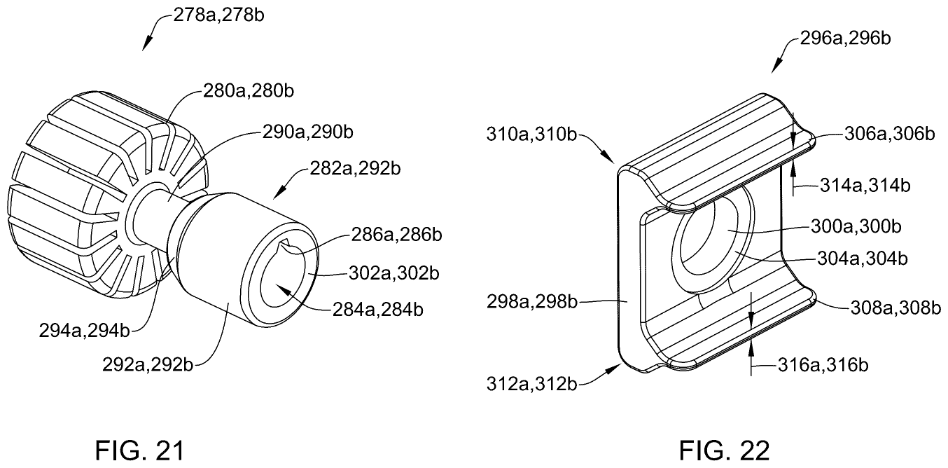

Referring additionally to FIG. 21, which is a perspective view of the illustrative push buttons 278a, 278b, the push buttons 278a, 278b may include an actuation member 280a, 280b and a stem 282a, 282b extending from the actuation member 280a, 280b. The actuation member 280a, 280b may include features configured to increase the gripability or friction thereof. For example, the actuation member 280a, 280b may include grooves, bumps, protrusions, slots, etc. along an outer surface thereof. In some cases, a lateral end surface of the actuation member 280a, 280b may include a concave recess 288b (see, for example, FIG. 20) configured to receive a user's finger. The stem 282a, 282b may include a lumen 284a, 284b extending at least partially therethrough. The lumen 284a, 284b may be configured to receive a mating mounting member 326a, 326b of the needle mount member 276. The internal side wall of the stem 282a, 282b may include a keyway such as a slot or groove 286a, 286b configured to receive a mating key or ridge 328a, 328b on the mounting member 326a, 326b of the needle mount member 276. The engagement of the ridge 328a, 328b with the slot 286a, 286b may allow the needle mount member 276 to rotate or pivot as the left and right push buttons 278a, 278b are rotated or pivoted.

The stem 282a, 282b may include a first region 290a, 290b connected to the actuation member 280a, 280b and having a first outer diameter, a second region 292a, 292b having a second outer diameter greater than the first outer diameter, and a third region 294a, 294b extending between the first region 290a, 290b and the second region 292a, 292b. The third region 294a, 294b may have an angled outer surface where the outer diameter varies along the length thereof. The outer diameter of the third region 294a, 294b may be sloped or tapered. The left and right push buttons 278a, 278b may be assembled with the vertical housing 238 such that the stem 282a, 282b is at least partially disposed within left and/or right lateral side openings 272a, 272b. For example, the actuation member 280a, 280b may be exterior to the vertical housing 238 while the second region 292a, 292b of the stem 282a, 282b may be disposed within the cavity 248 of the vertical housing 238.

In some embodiments, the first outer diameter of the first region 290a, 290b may be less than an axial length of the left and/or right lateral side openings 272a, 272b such that the first region 290a, 290b may move vertically within the left and/or right lateral side openings 272a, 272b. The second outer diameter of the second region 292a, 292b may be greater than the axial length of the left and/or right lateral side openings 272a, 272b to prevent the push buttons 278a, 278b from disengaging from the vertical housing 238. The sloped intermediate or third region 294a, 294b may allow the push buttons 278a, 278b to be partially actuated to allow for rotation of the push buttons 278a, 278b without vertical movement thereof, as will be described in more detail herein.

Returning to FIG. 20, the locking and actuation mechanism 274 may further include a left floating locking member 296a and a right floating locking member 296b. Generally, the left and right floating locking members 296a, 296b may act like spring-loaded pins that can move in and out of position to engage with other parts and create a locking action. The left and right floating locking members 296a, 296b may have a same structure. Referring additionally to FIG. 22 which is a perspective view of the illustrative floating locking members 296a, 296b, the floating locking members 296a, 296b may include a generally planar body portion 298a, 298b. An opening or aperture 300a, 300b may extend through an entirety of the body portion 298a, 298b. The mounting members 326a, 326b of the needle mount member 276 may be configured to extend through the opening 300a, 300b. In some cases, an annular recess 304a, 304b may be disposed around the opening 300a, 300b. The annular recess 304a, 304b may extend through less than entirety of the body portion 298a, 298b. When assembled with the push buttons 278a, 278b, an end surface 302a, 302b of the stem 282a, 282b may be configured to be disposed within the annular recess 304a, 304b. The floating locking members 296a, 296b may further include a first securement member 306a, 306b extending across a length thereof. The first securement member 306a, 306b may extend generally orthogonal from the body portion 298a, 298b and may be positioned adjacent to an upper end region 310a, 310b of the floating locking members 296a, 296b. The first securement member 306a, 306b may have a thickness 314a, 314b at a free end thereof that is less than the spacing 265, 267 between adjacent pins of the first or second plurality of pins 266a-o, 268a-o on the distal housing portion 244b or the pins of the proximal housing portion 244a. This may allow the first securement member 306a, 306b to be selectively positioned between adjacent pins. The floating locking members 296a, 296b may further include a second securement member 308a, 308b extending across a length thereof. The second securement member 308a, 308b may extend generally orthogonal from the body portion 298a, 298b and may be positioned adjacent to a lower end region 312a, 312b of the floating locking members 296a, 296b. The second securement member 308a, 308b may have a thickness 316a, 316b at a free end thereof that is less than the spacing 265, 267 between adjacent pins of the first or second plurality of pins 266a-o, 268a-o on the distal housing portion 244b or the pins of the proximal housing portion 244a. This may allow the second securement member 308a, 308b to be selectively positioned between adjacent pins. The first and second securement members 306a, 306b may extend from a same side of the body portion 298a, 298b. Further, the annular recess 300a, 300b may be positioned on a same side of the body portion 298a, 298b as the first and second securement members 306a, 306b.

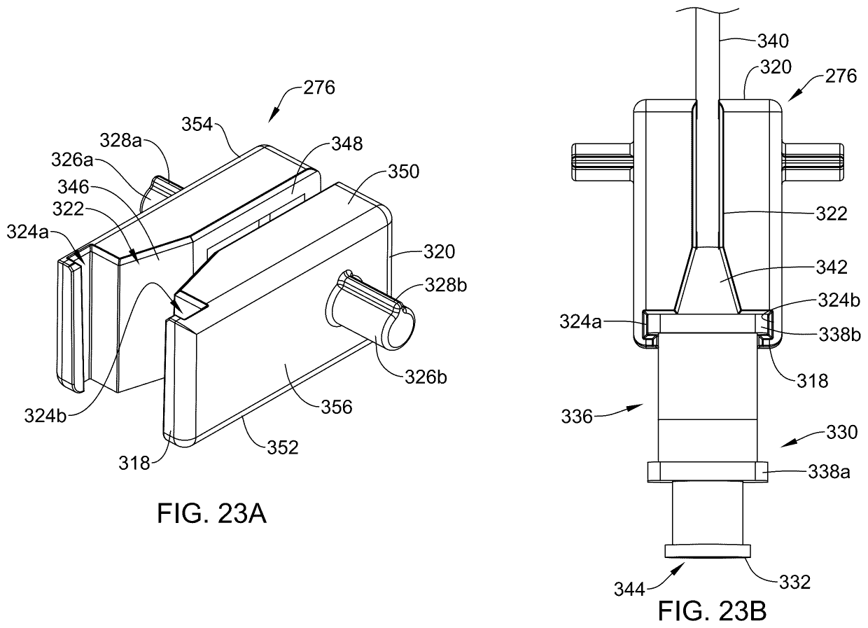

FIG. 23A is a perspective view of the illustrative needle mount member 276 and FIG. 23B is a top view of the needle mount member 276 with a guide needle 330 secured relative to the needle mount member 276. The needle mount member 276 may extend from a proximal end 318 to a distal end 320. A channel 322, configured to receive a portion of the guide needle 330, may extend from the proximal end 318 to the distal end 320. A profile of the channel 322 may vary from the proximal end 318 to the distal end 320. For example, the width of the channel 322 may mirror an outer surface or outer profile of the guide needle 330.

The guide needle 330 may extend from a proximal end 332 to distal end 334 (see, for example, FIG. 16). The distal end 334 may be configured to be inserted into the patient. The guide needle 330 may include a gripping region 336 adjacent to the proximal end 332 thereof. The gripping region 336 may include flanges 338a, 338b extending laterally therefrom to facilitate gripping of the guide needle 330. An elongate needle region 340 may have smaller profile than the gripping region 336 to facilitate insertion into the body. A transition region 342 may be positioned between the gripping region 336 and the elongate needle region 340. The transition region 342 may have a tapered outer profile. However, this is not required. A lumen 344 may extend from the proximal end 332 to the distal end 334 of the guide needle 330. The lumen 344 may be configured to receive a biopsy needle (not explicitly shown) therethrough. A diameter of the lumen 344 may reduce between the proximal end 332 and the distal end 334. In some cases, the diameter may reduce in a tapered or gradual manner. In other cases, the diameter may reduce in an abrupt or stair-step manner.

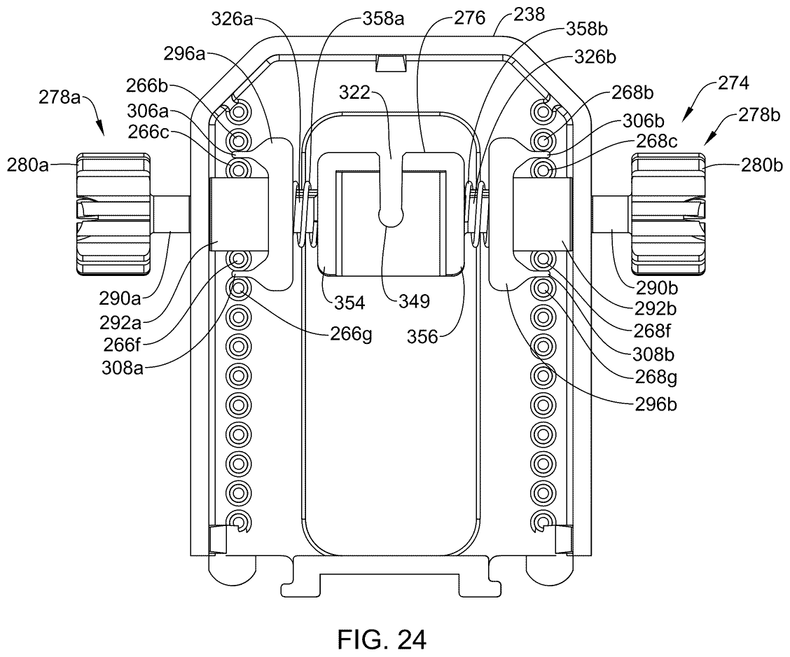

The channel 322 may include a left keyed region 324a and a right keyed region 324b for receiving a flange 338b of the guide needle 330. The channel 322 may include an angled region 346 positioned between the keyed regions 324a, 324b and a distal portion 348. The angled region 346 may reduce in width in the front to back direction and may be configured to receive the transition region 342 of the guide needle 330. The distal portion 348 of the channel 322 may have a relatively constant width that is sized to receive the elongate needle region 340 of the guide needle 330. In some examples, the distal portion 348 may be sized to limit or prevent lateral or left to right movement of the guide needle 330. The keyed regions 324a, 324b and the angled region 346 may extend from a top surface 350 to a bottom surface 352 of the needle mount member 276. The distal portion 348 may extend through less than an entire thickness of the needle mount member 276. For example, the distal portion 348 of the channel 322 may extend from the top surface 350 towards the bottom surface 352 and terminate before reaching the bottom surface 352. This may create a wall or ledge 349 (see, for example, FIG. 24) for the elongate needle region 340 to rest on.

The needle mount member 276 may further include a first or left mounting member 326a extending from a left lateral side 354 thereof and a second or right mounting member 326b extending from a right lateral side 356. The mounting members 326a, 326b may have a generally cylindrical shape configured to be received within the lumens 284a, 284b of the push buttons 278a, 278b. The mounting members 326a, 326b may include a mating key or ridge 328a, 328b configured to align with and be received within the slot 286a, 286b extending within the lumens 284a, 284b of the push buttons 278a, 278b.

The locking and actuation mechanism 274 may be movable between a locked first configuration in which both vertical movement of the needle mount member 276 and swivel movement of the needle mount member 276 are prevented, a partially locked second configuration in which vertical movement of the needle mount member 276 is prevented while allowing swivel movement of the needle mount member 276, and an unlocked third configuration in which both vertical movement of the needle mount member 276 and swivel movement needle mount member 276 are allowed.

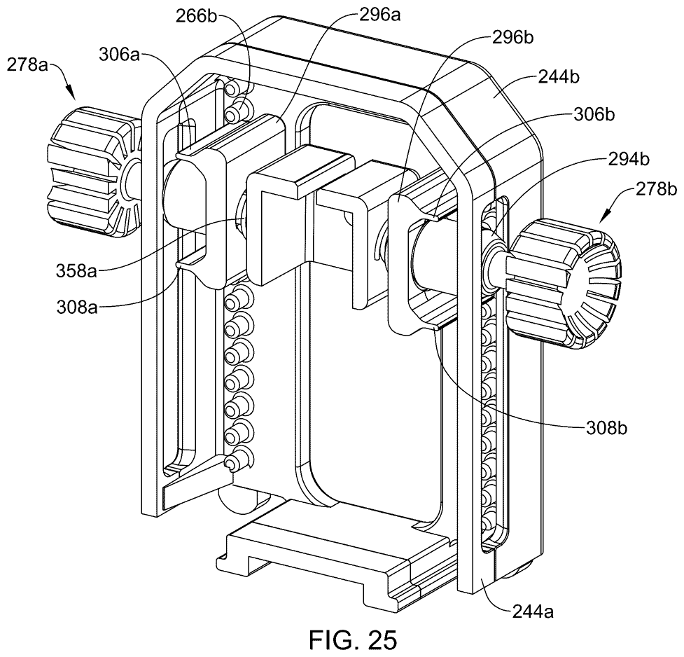

FIG. 24 is a partial cross-sectional view of the vertical housing 238 with the locking and actuation mechanism 274 and needle mount member 276 disposed therein and in the first or fully locked configuration. FIG. 25 is a perspective view of the partial cross-sectional view of FIG. 24. The left floating locking member 296a may be positioned over the left mounting member 326a of the needle mount member 276 with the first and second securement members 306a, 308a oriented laterally outwards (e.g., towards the left lateral side of the vertical housing 238. The right floating locking member 296b may be positioned over the right mounting member 326b of the needle mount member 276 with the first and second securement members 306b, 308b oriented laterally outwards (e.g., towards the right lateral side of the vertical housing 238. A first or left biasing member 358a may be positioned between the left lateral side 354 of the needle mount member 276 and the laterally inward surface of the left floating locking member 296a and a second or right biasing member 358b may be positioned between the right lateral side 356 of the needle mount member 276 and the laterally inward surface of the right floating locking member 296b. In one example, the biasing members 358a, 358b may be compression springs, wave spring washers, leaf springs, or the like. However, this is not required. The biasing members 358a, 358b may be resilient members configured to exert a laterally outward biasing force on the floating locking members 296a, 296b. The biasing force of the biasing members 358a, 358b may be temporarily overcome by applying a laterally inward force to the push buttons 278a, 278b, such as, for example, by a user pinching the push buttons 278a, 278b. The push buttons 278a, 278b may be moved between a laterally outward most position to a laterally inward most position or a location therebetween. When the laterally inward force is removed, the biasing members 358a, 358b return to their original configuration.

In the first or fully locked configuration, the push buttons 278a, 278b are free from a laterally inward biasing force and the biasing members 358a, 358b are pushing the floating locking members 296a, 296b laterally outwards. As the floating locking members 296a, 296b are moved laterally outwards, the securement members 306a, 306b, 308a, 308b are positioned in the gap between adjacent pins. For example, the first securement member 306a of the left floating locking member 296a is positioned between pin 266b and pin 266c (as well as corresponding pins on the proximal housing portion 244a) and the second securement member 308a of the left floating locking member 296a is positioned between pin 266f and pin 266g (as well as corresponding pins on the proximal housing portion 244a). Similarly, the first securement member 306b of the right floating locking member 296b is positioned between pin 268b and pin 268c (as well as corresponding pins on the proximal housing portion 244a) and the second securement member 308b of the right floating locking member 296b is positioned between pin 268f and pin 268g. The mechanical engagement between the securement members 306a, 306b, 308a, 308b and the pins 266b, 266c, 268b, 268c, 266f, 266g, 268f, 268g prevent the locking and actuation mechanism 274 from being moved vertically within the vertical housing 238. For example, if a user were to grip one or both of the push buttons 278a, 278b and try to vertically displace the push buttons 278a, 278b (and hence the needle mount member 276), upward movement would be prevented by pins 266b, 268b, 266f, 268f and downward movement would be prevented by pins 266c, 268c, 266g, 268g. Further, the biasing members 358a, 358b may bias the intermediate region 294a, 294b of the stems 282a, 282b into the left and/or right lateral side openings 272a, 272b of the vertical housing 238 to form a friction fit (see, for example, FIG. 25). This may prevent the push buttons 278a, 278b from being rotated in a clockwise or counterclockwise direction (about a longitudinal axis of the push buttons 278a, 278b). Thus, vertical and angular movement of the needle mount member 276 may be prevented.

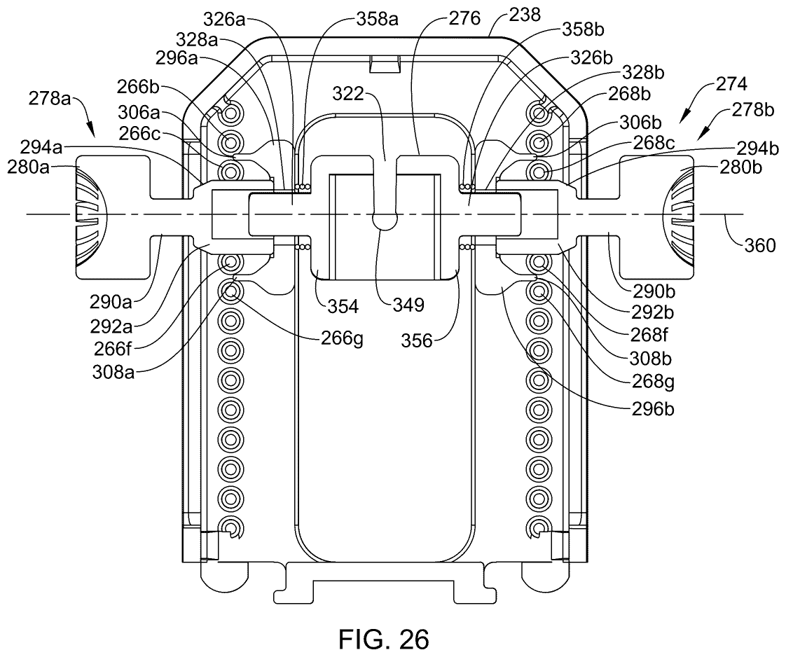

FIG. 26 is a partial cross-sectional view of the of the vertical housing 238 with the locking and actuation mechanism 274 and needle mount member 276 disposed therein and in the second or partially locked configuration. When the locking and actuation mechanism 274 is in the second, partially locked, configuration, the push buttons 278a, 278b are moved laterally inwards to a point between the laterally outward most position and the laterally inward most position. For example, the push buttons 278a, 278b may be moved laterally inward just enough to remove the frictional engagement between the intermediate region 294a, 294b of the stems 282a, 282b and the walls of the left and/or right lateral side openings 272a, 272b. In this configuration, the mechanical engagement between the securement members 306a, 306b, 308a, 308b and the pins 266b, 266c, 268b, 268c, 266f, 266g, 268f, 268g remains. However, as the push buttons 278a, 278b are not mechanically secured to the floating locking members 296a, 296b, the push buttons 278a, 278b may be rotated independently of the floating locking members 296a, 296b. For example, the push buttons 278a, 278b may be rotated about their lateral axis 360. The rotational force may be translated to the needle mount member 276 via the mating slot 286a, 286b and ridge 328a, 328b arrangement between the push buttons 278a, 278b and the mounting members 326a, 326b of the needle mount member 276 causing the needle mount member 276 to pivot about the axis 360. As the guide needle 330 is removably coupled to the needle mount member 276, as the needle mount member 276 pivots the guide needle 330 may also pivot.

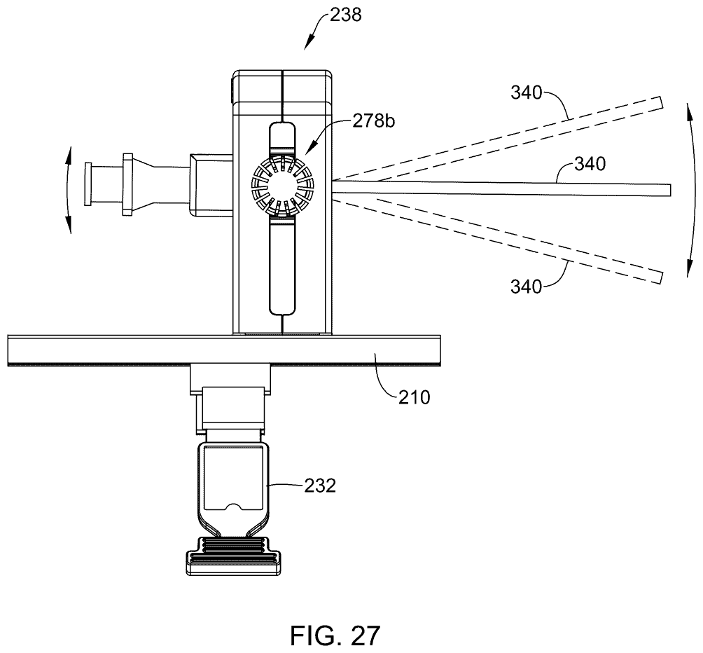

FIG. 27 is a schematic side view of the of the guide system 200 illustrating the pivotable arrangement of the guide needle 330 and the needle mount member 276. For example, the push buttons 278a, 278b and the needle mount member 276 may be rotated to allow the elongate needle region 340 of the guide needle 330 to extend at an angle parallel and non-parallel to the horizontal axis 202. For example, FIG. 27 illustrates some alternative positions of the guide needle 330 and needle mount member 276. It is contemplated that the extent to which the guide needle 330 may be offset (up or down) from parallel to the horizontal axis may be determined at least in part by a vertical location of the needle mount member 276. For example, upward angulation may be limited by the vertical housing and downward angulation may be limited by the horizontal slide member 210.

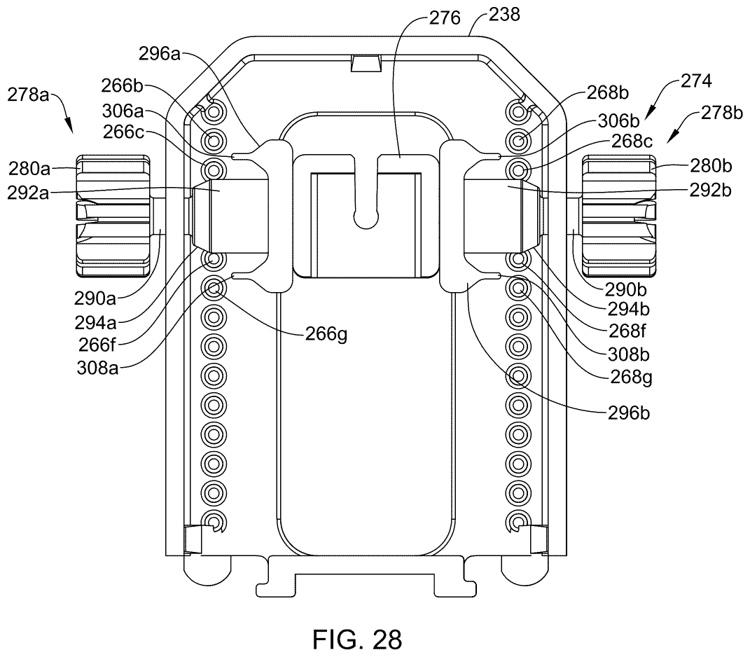

FIG. 28 is a partial cross-sectional view of the vertical housing 238 with the locking and actuation mechanism 274 and needle mount member 276 disposed therein and in the third or fully unlocked configuration. When the locking and actuation mechanism 274 is in the third configuration, the push buttons 278a, 278b are moved laterally inwards to the laterally inward most position. The push buttons 278a, 278b displace the left and right floating locking members 296a, 296b laterally inwards. As the left and right floating locking members 296a, 296b are moved laterally inwards, the securement members 306a, 306b, 308a, 308b are moved out of the gaps between the pins 266b, 266c, 268b, 268c, 266f, 266g, 268f, 268g. The locking and actuation mechanism 274 and the needle mount member 276 may then be moved vertically up or down (e.g., along the vertical axis 206) within the vertical housing 238. Further, the locking and actuation mechanism 274 and the needle mount member 276 may be rotated as described with respect to FIGS. 26 and 27 to adjust an orientation of the guide needle 330. The height of the openings 250, 260 may limit upwards and downwards movement of the needle mount member 276 and the locking and actuation mechanism 274. When the locking and actuation mechanism 274 and needle mount member 276 are at the desired vertical height, the biasing force on the push buttons 278a, 278b may be released. The biasing mechanisms 358a, 358b may bias the push buttons 278a, 278b and the floating locking members 296a, 296b laterally outwards. If the securement members 306a, 306b, 308a, 308b are not directly aligned with a gap between adjacent pins, the curved outer surface of the pins may direct the securement members 306a, 306b, 308a, 308b into the closest gap.

In use, the guide system 200 may be secured to an ultrasound probe using the strap 232 before or after the ultrasound probe is positioned within the anatomy. For example, for a prostate biopsy, the ultrasound probe may be positioned within the rectum. Once the guide system 200 is secured to the ultrasound probe and the ultrasound probe is positioned, the vertical housing 238 may be axially displaced along the horizontal slide member 210 and secured in the desired axial position. This may be performed with the locking and actuation mechanism 274 in the first (locked), second (partially locked), or third (unlocked) configurations. Next, a vertical position of the needle mount member 276 (and thus a vertical position of the guide needle 330) may be adjusted. The locking and actuation mechanism 274 may be moved to the third or unlocked configuration to allow the needle mount member 276 to be moved vertically within the cavity 248 of the vertical housing 238. Once the needle mount member 276 is at the desired height or vertical location, the locking and actuation mechanism 274 may be moved to the second or partially locked configuration to lock the vertical position of the needle mount member 276. In some cases, the locking and actuation mechanism 274 may be moved to the first configuration to lock the vertical position of the needle mount member 276 before depressing the push buttons 278a, 278b to the second configuration to adjust an angle of the guide needle 330. In the second or partially locked configuration, the angle of the needle mount member 276 may be adjusted to position the guide needle 330 at a desired angle. Once the needle mount member 276 is in the desired position, the locking and actuation mechanism 274 may be moved to the first or locked configuration to lock or secure the needle mount member 276 at the desired angle.

It is contemplated that multiple samples may be taken with the needle mount member 276 at a same height. For example, the angle of the guide needle 330 may be adjusted to obtain multiple samples within a same plane. It is further contemplated that the order of the steps described herein may be rearranged. For example, the vertical and/or angular position of the guide needle 330 may be adjusted prior to moving the vertical housing 238 along the horizontal slide member 210 towards or away from the patient's body.

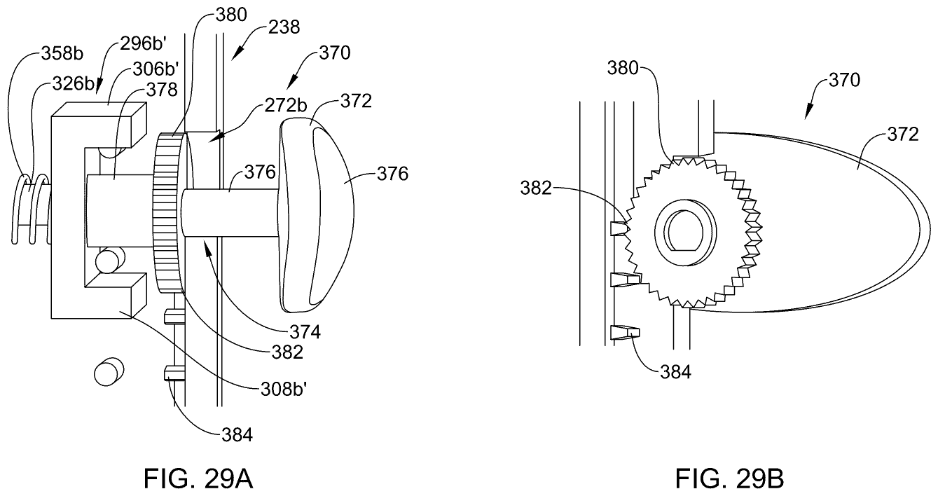

FIG. 29A is a side view of another illustrative push button 370 that may be used with the guide system 200 with the vertical housing 238 in partial cross-section. FIG. 29B is a perspective view of the illustrative push button 370 in partial cross-section with the vertical housing 238 in partial cross-section. While FIGS. 29A and 29B illustrate only a single push button 370, it should be understood a second push button may be provided on the opposing lateral side of the vertical housing 238. The second push button may interact with the needle mount member 276 in a similar manner to the first push button 370. The push button 370 may include an actuation member 372 and a stem 374 extending from the actuation member 372. The actuation member 372 may include features configured to increase the gripability or friction thereof. For example, the actuation member 372 may include grooves, bumps, protrusions, slots, etc. along an outer surface thereof. In some cases, a lateral end surface of the actuation member 372 may include a concave recess 376 configured to receive a user's finger. The stem 374 may include a lumen (not explicitly shown) extending at least partially therethrough. The lumen may be configured to receive a mating mounting member 326a of the needle mount member 276. The internal side wall of the stem 374 may include a keyway such as a slot or groove (similar in form and function slots 286a, 286b) configured to receive a mating key or ridge 328b on the mounting member 326b of the needle mount member 276. The engagement of the ridge 328b with the slot may allow the needle mount member 276 to rotate or pivot as the left and right push button 370 are rotated or pivoted.

The stem 374 may include a first region 376 connected to the actuation member 372 and having a first outer diameter, a second region 378 having a second outer diameter greater than the first outer diameter, and a third region 380 extending between the first region 376 and the second region 378. The third region 380 may be a gear having a plurality of grooves and ridges or teeth 382 extending from an outer surface thereof. The teeth 382 may be configured to engage mating grooves and ridges or teeth 384 on the vertical housing 238. The teeth 384 may extend laterally inwards from a laterally inwards surface of the lateral side walls 246b, 246c, 258b, 258c of front and back housing portions 244a, 244b. The left and right push button 370 may be assembled with the vertical housing 238 such that the stem 374 is at least partially disposed within left and/or right lateral side openings 272a, 272b. For example, the actuation member 372 may be exterior to the vertical housing 238 while the second region 378 of the stem 374 may be disposed within the cavity 248 of the vertical housing 238.

In some embodiments, the first outer diameter of the first region 376 may be less than an axial length of the left and/or right lateral side openings 272a, 272b such that the first region 376 may move vertically within the left and/or right lateral side openings 272a, 272b. The second outer diameter of the second region 378 may be greater than the axial length of the left and/or right lateral side openings 272a, 272b to prevent the push button 370 from disengaging from the vertical housing 238. The third region 380 may allow the push button 370 to be partially actuated to allow for rotation of the push button 370 without vertical movement thereof.

Vertical movement of the locking and actuation mechanism 274 and the needle mount member 276 may be achieved in manner similar to that described herein. For example, the locking and actuation mechanism 274 may be movable between a locked first configuration in which both vertical movement of the needle mount member 276 and swivel movement of the needle mount member 276 are prevented, a partially locked second configuration in which vertical movement of the needle mount member 276 is prevented while allowing swivel movement of the needle mount member 276, and an unlocked third configuration in which both vertical movement of the needle mount member 276 and swivel movement needle mount member 276 are allowed. FIGS. 29A and 29B illustrate the push button 370 in the locked or first configuration. Here, the biasing member 358b biases or pushes the floating locking member 296b′ laterally outwards such that the securement members 306b′, 308b′ positioned between pins. The laterally outward force of the biasing member 358b may also bias the gear 380 against the laterally inward surface of the right lateral side wall 258c. In this position, one or more of the teeth 382 of the push button 370 engage one or more of the teeth 384 of the vertical housing 238, as shown in FIG. 29B. When the teeth 382, 384 are engaged, rotation or pivoting of the push button 370 is precluded.

To move the locking and actuation mechanism 274 to the second, partially locked, configuration, the push button 370 is moved laterally inwards to a point between a laterally outward most position (first locked configuration) and a laterally inward most position (third fully unlocked configuration). For example, the push button 370 may be moved laterally inward to laterally space the gear 380 of the push button 370 from the teeth 384 of the vertical housing 238. In this configuration, the mechanical engagement between the securement members 306b', 308b′ and the pins remains. However, as the push button 370 is not mechanically secured to the floating locking member 296b′, the push button 370 may be rotated independently of the floating locking member 296b′. For example, the push button 370 may be rotated about its longitudinal axis when the teeth 382 of the gear 380 are disengaged from the teeth 384 of the vertical housing 238. The rotational force may be translated to the needle mount member 276 via the mating slot and ridge 328b arrangement between the needle mount member 276 and the mounting members 326a, 326b of the needle mount member 276 causing the needle mount member 276 to pivot about the longitudinal axis of the push button 370. As the guide needle 330 is removably coupled to the needle mount member 276, as the needle mount member 276 pivots the guide needle 330 may also pivot. To secure the needle mount member 276 at the desired angular offset, the laterally inward force on the push button 370 may be released and the teeth 382 of the gear 380 may mechanically engage the teeth 384 on the vertical housing 238 to prevent further rotation.

To move the locking and actuation mechanism 274 to the third configuration, the push button 370 is moved laterally inwards to the laterally inward most position. The push button 370 displaces the right floating locking member 296b′ laterally inwards. As the right floating locking member 296b′ is moved laterally inwards, the securement members 306b', 308b′ are moved out of the gaps between the pins. The locking and actuation mechanism 274 and the needle mount member 276 may then be moved vertically (e.g., along the vertical axis 206) within the vertical housing 238. Further, the locking and actuation mechanism 274 and the needle mount member 276 may be rotated as described herein to adjust an orientation of the guide needle 330. The height of the openings 250, 260 may limit upwards and downwards movement of the needle mount member 276 and the locking and actuation mechanism 274. When the locking and actuation mechanism 274 and needle mount member 276 are at the desired vertical height, the biasing force on the push button 370 may be released. The biasing mechanism 258b may bias the push button 370 and the floating locking member 296b′ laterally outwards. If the securement members 306b′, 308b′ are not directly aligned with a gap between adjacent pins, the curved outer surface of the pins may direct the securement members 306b′, 308b′ into the closest gap.

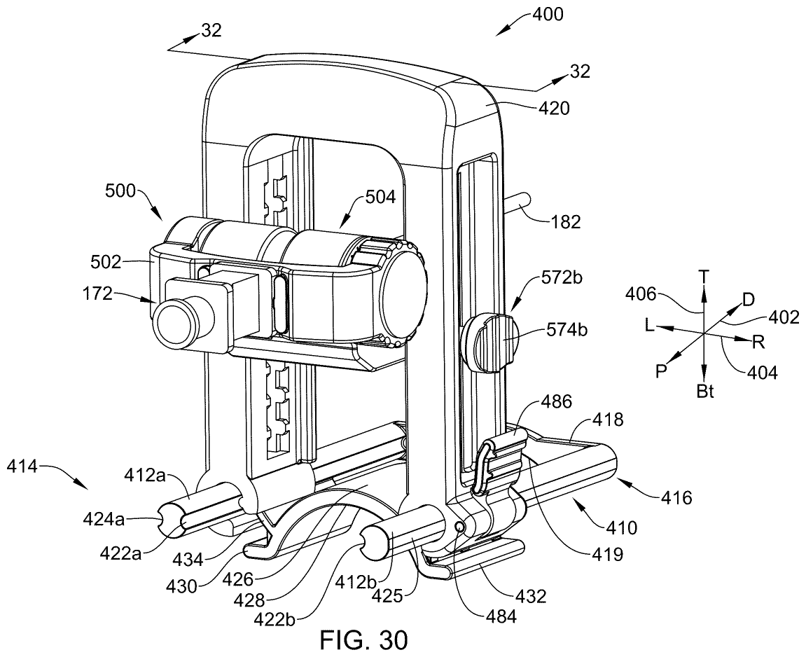

FIG. 30 is a perspective view an illustrative prostate biopsy guide system 400 in a first configuration. While the guide system 400 is described with respect to prostate biopsy, it is contemplated that the guide system 400 may be used in other procedures. In one illustrative example, the guide system 400 may be used to facilitate the injection of a spacing material, such as between the rectum and the prostate to prevent damage during radiation therapy. One illustrative system for injecting materials into the space between the rectum and the prostate is the SpaceOAR Vue™ System available from Boston Scientific Corporation, Marlborough, MA, USA. The guide system 400 may be configured to allow for horizontal, vertical, and angular movement of the biopsy needle (not explicitly shown) while also fixing the biopsy needle in a particular configuration. The guide system 400 may be configured to mount to an ultrasound probe (not explicitly shown) such that the guide system 400 is in a generally fixed position relative to the ultrasound probe. As used herein, movement in the horizontal direction or relational positioning in a direction generally parallel to axis 402, will be described as axial movement or an axial direction with relational positioning using proximal (P) (or front) and distal (D) (or back); movement or relational positioning in a direction generally parallel to axis 404 will be described as lateral movement or a lateral direction with relational positioning using left (L) and right (R); and movement or relational positioning in a direction generally parallel to axis 406 will be described as vertical movement or a vertical direction with relational positioning using top (T) or up and bottom (Bt) or down. The use of the terms “proximal”, “distal”, “front,” “back”, “left”, “right”, “top”, “bottom”, “up”, and “down” are not intended to limit the guide system 400 to a particular orientation, but rather facilitate discussion of relative orientation.

Generally, the guide system 400 may be configured to control a horizontal position, a vertical position, and/or an angle of a guide needle. The guide system 400 may be configured to lock the guide needle 172 (as shown and described with respect to FIG. 9) in a desired orientation independent of an ultrasound probe. It is contemplated that guide needles having alternative configurations may also be used with the guide system 400. Further, the vertical and/or angular position may be held as the guide needle is moved towards the patient's body and/or away from the patient's body. The guide system 400 may include a horizontal slide member 410, a vertical support 420, a locking mechanism 486, a needle mount member 502, and a gear assembly member 504, among other features. The needle mount member 502 may be pivotable relative to the gear assembly member 504 to change an angle of the guide needle 172. Further, the needle actuation assembly 500 may be configured to move up and down along the vertical support 420 to control a vertical position of the needle mount member 502 (and thus the guide needle 172). The guide system 400 may include locking features to selectively lock a vertical position of the needle actuation assembly 500 and an angular position of the needle mount member 502. The vertical support 420 may be moved along the horizontal slide member 410 to move the guide needle 172 distally towards and/or proximally away from the patient. The vertical support 420 may be selectively secured to the horizontal slide member 410 to lock the vertical support 420 in a desired axial position.

The horizontal slide member 410 may include a first, or left, rail 412a and a second, or right, rail 412b. The left and right rails 412a, 412b may be laterally spaced and extend generally parallel to one another. The left and right rails 412a, 412b may extend from a first or proximal region 414 of the horizontal slide member 410 to a second or distal region 416 (of the horizontal slide member 410. A third rail or crossbar 418 may extend between the left and right rails 412a, 412b adjacent the distal region 416 thereof. The crossbar 418 may hold the left and right rails 412a, 412b in a laterally spaced configuration and limit horizontal (front to back) movement of a vertical frame or vertical support 420. The crossbar 418 may include a concave lower or bottom surface 419 configured to conform to an outer surface of the ultrasound probe. For example, the concave lower surface 419 may rest on an outer surface of the ultrasound probe. In some embodiments, the crossbar 418 may be formed as single monolithic structure with the left and right rails 412a, 412b. In other embodiments, the crossbar 418 may be formed as a separate component from the first and/or second rails 412a, 412b and subsequently coupled with the first and/or second rails 412a, 412b.

The left and right rails 412a, 412b may each include a first axially extending slot 422a, 422b. The first axially extending slots 422a, 422b may be oriented laterally inwards on the left and right rails 412a, 412b and thus may be positioned on opposing side of the respective rail 412a, 412b. The left rail 412a may include a second axially extending slot 424a. The second axially extending slot 424a may be oriented laterally outwards on the left rail 412a.

The slots 422a, 422b, 424a may extend along an entirety of the axial length of the rails 412a, 412b or less than an entirety of the length, as desired. It is contemplated that the distance of travel of the vertical support 420 may be determined by a length of the slots 422a, 422b, 424a. In some examples, the vertical support 420 may be secured relative to the horizontal slide member 410 such that the axial location of the vertical support 420 is infinitely adjustable.

The right rail 412b may include an axially extending flattened region 425. The flattened region 425 may be an axially extending region where the curvature of the right rail 412b has been removed. This may create a region where a locking mechanism 486 may frictionally engage with the right rail 412b to selectively lock the vertical support 420 at a location along the horizontal slide member 410, as will be described in more detail herein.

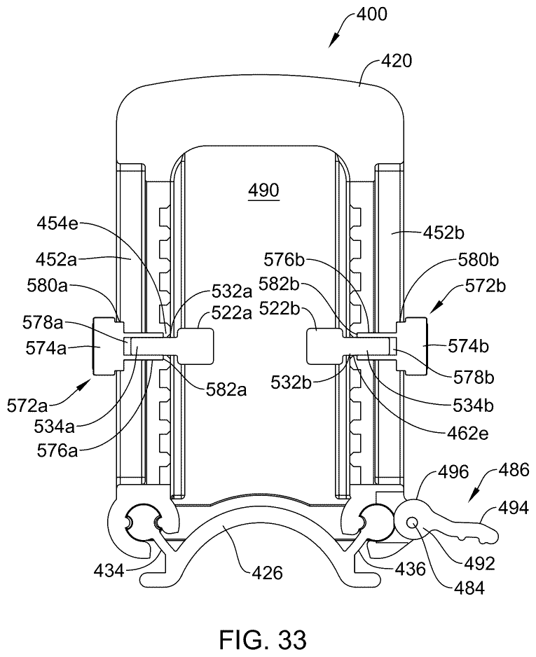

The horizontal slide member 410 may further include a probe mount 426 positioned adjacent to the proximal region 414 and extending between the left and right rails 412a, 412b. In some embodiments, the probe mount 426 may be formed as single monolithic structure with the left and right rails 412a, 412b. In other embodiments, the probe mount 426 may be formed as a separate component from the first and/or second rails 412a, 412b and subsequently coupled with the left and/or right rails 412a, 412b. The probe mount 426 may include a concave lower or bottom surface 428 configured to conform to an outer surface of the ultrasound probe. For example, the concave lower surface 428 may rest on an outer surface of the ultrasound probe. Said differently, the probe mount 426 may include an arch or a curve extending between the left and right rails 412a, 412b. The probe mount 426 may extend from a first or left lateral end 430 to a second or right lateral end 432. The probe mount 426 may be connected to the left rail 412a via a first or left connection member 434 extending between a curved intermediate region of the probe mount 426 and the left rail 412a. Similarly, the probe mount 426 may be connected to the right rail 412b via a second or right connection member 436 (see, for example, FIG. 33) extending between a curved intermediate region of the probe mount 426 and the right rail 412b. The left and right lateral ends 430, 432 of the probe mount 426 may extend laterally beyond the left and right rails 412a, 412b, respectively. While not explicitly shown, the probe mount 426 may include features configured to releasably secure a strap (such as straps 46, 232) thereto similar in form and function to those described herein.

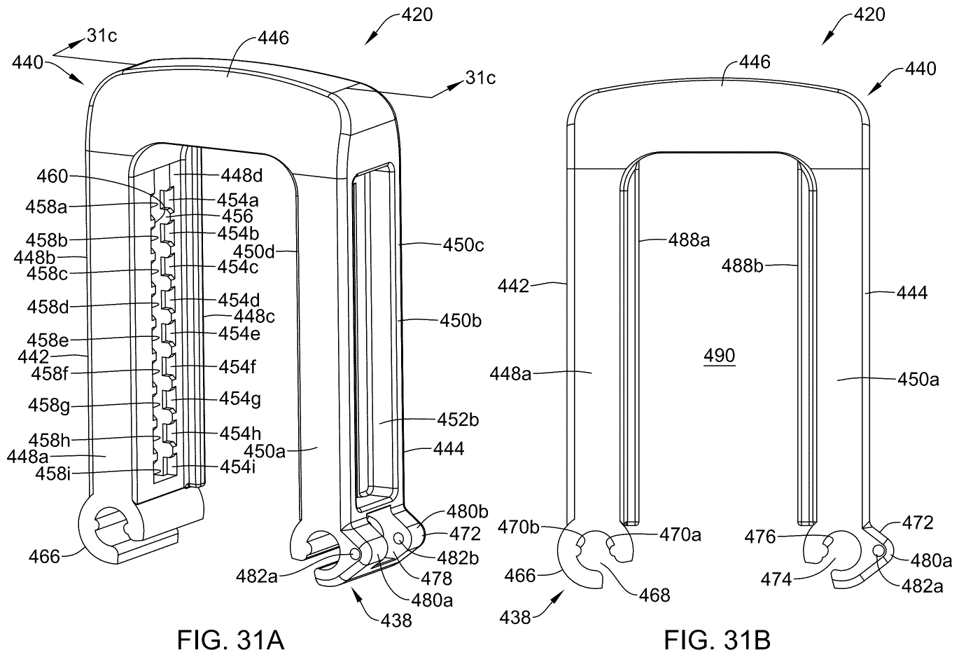

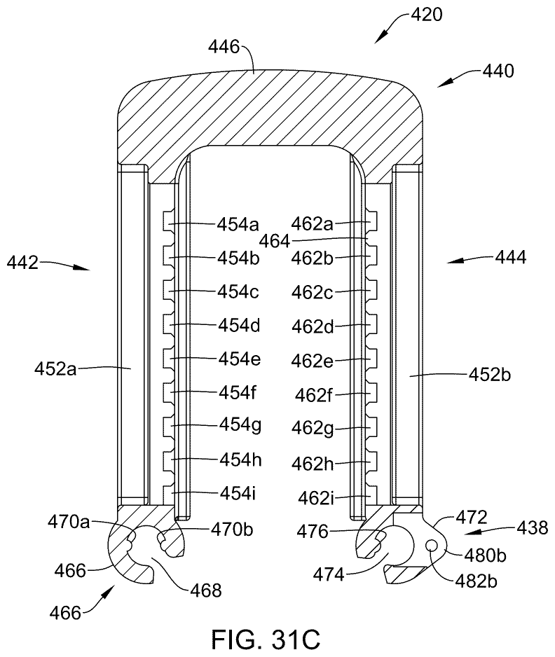

The vertical support 420 may be configured to be axially displaced along the horizontal slide member 410. FIG. 31A is a perspective view of an illustrative vertical support 420 and FIG. 31B is a front view of the illustrative vertical support 420. The vertical support 420 may extend from a bottom end region 438 to a top end region 440. The vertical support 420 may be formed as a single monolithic structure or as two or more components coupled together. The vertical support 420 may have a generally inverted “U” shape having a first or left arm 442 a second or right arm 444 laterally spaced from the left arm 442, and an interconnecting arm 446 extending between a top region 440 of the left arm 442 and the right arm 444.

The left arm 442 and the right arm 444 may extend generally parallel to one another. The interconnecting arm 446 may extend generally orthogonal to the left arm 442 and the right arm 444. The left arm 442 may include a proximal face 448a, a left (or laterally outward face) 448b, a distal face 448c, and a right (or laterally inward face) 448d. The right arm 444 may include a proximal face 450a, a right (or laterally outward face) 450b, a distal face 450c, and a left (or laterally inward face) 450d.

The left arm 442 may include a first channel 452a extending from the laterally outward face 448b to the laterally inward face 448d. The first channel 452a may have a first axial length at the laterally outward face 448b and a second axial length at the laterally inward face 448d. The second axial length may be less than the first axial length. In some examples, a height of the first channel 452a may be greater at the laterally outward face 448b than a height of the first channel 452a at the laterally inward face 448d. The right arm 444 may include a second channel 452b extending from the laterally outward face 450b to the laterally inward face 450d. The second channel 452b may have a first axial length at the laterally outward face 450b and a second axial length at the laterally inward face 450d. The second axial length may be less than the first axial length. In some examples, a height of the second channel 452b may be greater at the laterally outward face 450b than a height of the second channel 452b at the laterally inward face 450d.

Referring additionally to FIG. 31C, which is a cross-sectional view of the vertical support 420 taken at line 31C-31C of FIG. 31A, the left arm 442 further include a first plurality of notches 454a-i extending from the laterally inward face 448d towards the laterally outward face 448b. The first plurality of notches 454a-i may extend through less than a lateral width of the left arm 442. The first plurality of notches 454a-i may be positioned adjacent to the back side of the first channel 452a. For example, the first plurality of notches 454a-i may be formed in a back wall defining the first channel 452a. The first plurality of notches 454a-i may be separated from one another by a height 456. The first plurality of notches 454a-i may extend in a vertical array parallel to and adjacent to the laterally inward face 448d. The first plurality of notches 454a-i may be uniformly spaced from one another.

The left arm 442 further include a second plurality of notches 458a-i extending from the laterally inward face 448d towards the laterally outward face 448b. The second plurality of notches 458a-i may extend through less than a lateral width of the left arm 442. The second plurality of notches 458a-i may be positioned adjacent to the front or proximal side of the first channel 452a. For example, the second plurality of notches 458a-i may be formed in a front wall defining the first channel 452a. The second plurality of notches 458a-i may be separated from one another by a height 460. The second plurality of notches 458a-i may extend in a vertical array parallel to and adjacent to the laterally inward face 448d. The second plurality of notches 458a-i may be uniformly spaced from one another. Further, the second plurality of notches 458a-i may be vertically aligned with a corresponding notch of the first plurality of notches 454a-i.

The right arm 444 further include a first plurality of notches 462a-i extending from the laterally inward face 450 towards the laterally outward face 450b. The first plurality of notches 462a-i may extend through less than a lateral width of the right arm 444. The first plurality of notches 462a-i may be positioned adjacent to the back side of the second channel 452b. For example, the first plurality of notches 462a-i may be formed in a back wall defining the second channel 452b. The first plurality of notches 462a-i may be separated from one another by a height 464. The first plurality of notches 462a-i may extend in a vertical array parallel to and adjacent to the laterally inward face 450d. The first plurality of notches 462a-i may be uniformly spaced from one another.

While not explicitly shown, the right arm 444 further include a second plurality of notches extending from the laterally inward face 450d towards the laterally outward face 450b. The second plurality of notches may extend through less than a lateral width of the right arm 444. The second plurality of notches may be positioned adjacent to the front or proximal side of the second channel 452b. For example, the second plurality of notches may be formed in a front wall defining the second channel 452b. The second plurality of notches may be separated from one another by a height. The second plurality of notches may extend in a vertical array parallel to and adjacent to the laterally inward face 450d. The second plurality of notches may be uniformly spaced from one another. Further, the second plurality of notches may be vertically aligned with a corresponding notch of the first plurality of notches 462a-i. The notches in the right arm 444 may be vertically aligned with a corresponding notch in the left arm 442. The right arm 444 may include a same number of notches as the left arm 442.

While the illustrated embodiment includes nine notches 454a-i, 458a-i, 462a-i in each array of notches, there may be fewer than nine or more than nine positioning pins notches 454a-i, 458a-i, 462a-i, as desired. It is contemplated that increasing the number of notches in each array 454a-i, 458a-i, 462a-i may increase a number of possible vertical height locations of the needle mount member 276 while decreasing the number of notches in each array 454a-i, 458a-i, 462a-i may decrease a number of possible vertical height locations of the needle mount member 502. Further, while the first and second plurality of notches 454a-i, 458a-i, 462a-i are illustrated as having a generally rectangular prism shape, the first and second plurality of notches 454a-i, 458a-i, 462a-i may take other shapes, as desired. Collectively, the first and second plurality of notches 454a-i, 458a-i of the left arm 442 and the first and second plurality of notches 462a-i of the right arm 444 may form a rack for receiving a protrusion 532a, 532b of a gear assembly member 504. When the protrusion 532a, 532b is disposed between within the notches, vertical adjustment of the needle actuation assembly 500 may be precluded. The protrusion 532a, 532b may be selectively displaced to allow the needle actuation assembly 500 to be moved up and down along the vertical support 420, as will be described in more detail herein.

The left arm 442 may further include a first or left generally C-shaped bracket 466 positioned along a bottom region 438 thereof. The bracket 466 may extend between the front and back of the left arm 442. The C-shaped bracket 466 may define a left axially extending channel 468. The left C-shaped bracket 466 may be configured to slidably disposed over the left rail 412a of the horizontal slide member 410. A first protrusion 470a may extend from an inner wall of the left C-shaped bracket 472 and into the channel 474. The protrusion 470a may extend along an entire length of the left C-shaped bracket 466 or less than an entire length thereof, as desired. The first protrusion 470a may be received within first axially extending slot 422a of the left rail 412a. A second protrusion 470b opposite from the first protrusion 470a may extend from an inner wall of the left C-shaped bracket 466 and into the channel 468. The second protrusion 470b may extend along an entire length of the left C-shaped bracket 466 or less than an entire length thereof, as desired. The second protrusion 470b may be received within the second axially extending slot 424a of the left rail 412a.

The right arm 444 may further include a second or right generally C-shaped bracket 472 positioned along a bottom region 438 thereof. The bracket 472 may extend between the front and back of the left arm 442. The C-shaped bracket 472 may define a right axially extending channel 474. The right C-shaped bracket 472 may be configured to slidably disposed over the right rail 412b of the horizontal slide member 410. A first protrusion 476 may extend from an inner wall of left C-shaped bracket 472 and into the channel 474. The protrusion 476 may extend along an entire length of the left C-shaped bracket 472 or less than an entire length thereof, as desired. The first protrusion 476 may be received within first axially extending slot 422b of the right rail 412b. The right C-shaped bracket 472 may include an opening 478 extending through laterally outer portion of the right C-shaped bracket 472. The opening 478 may extend through a side wall of the right C-shaped bracket 472 such that the channel 474 is laterally accessible. The opening 478 may extend less than entire length of the right C-shaped bracket 472. A first hinge mount 480a may extend from the right C-shaped bracket 472 adjacent to a proximal end thereof and a second hinge mount 480b may extend from the right C-shaped bracket 472 adjacent to a back end thereof. The first and second hinge mounts 480a, 480b may be positioned on opposing ends of the opening 478. A first aperture 482a may extend through an axial length of the first hinge mount 480a and a second aperture 482b may extend through an axial length of the second hinge mount 480b. The first and second apertures 482a, 482b may be configured to receive a pin 484 (see, for example, FIGS. 30, 32, and 33) therethrough for pivotably or hingedly coupling a locking mechanism 486 to the right C-shaped bracket 472. In some cases, the features configured to engage the locking mechanism 486 may be provided additionally or alternatively at the left generally C-shaped bracket 466.

The vertical support 420 may further include a first laterally inwardly extending wall 488a and a second laterally inwardly extending wall 488b. The laterally inwardly extending walls 488a, 488b may extend into a channel 490 defined by the left arm 442, right arm 444, and interconnecting arm 446 of the vertical support 420. The laterally inwardly extending walls 488a, 488b may be axially offset within the vertical support 420 such that the laterally inwardly extending walls 488a, 488b are positioned closer to the back of the vertical support 420. However, this is not required. In some cases, the laterally inwardly extending walls 488a, 488b may provide a mechanical stop for the gear assembly member 504.

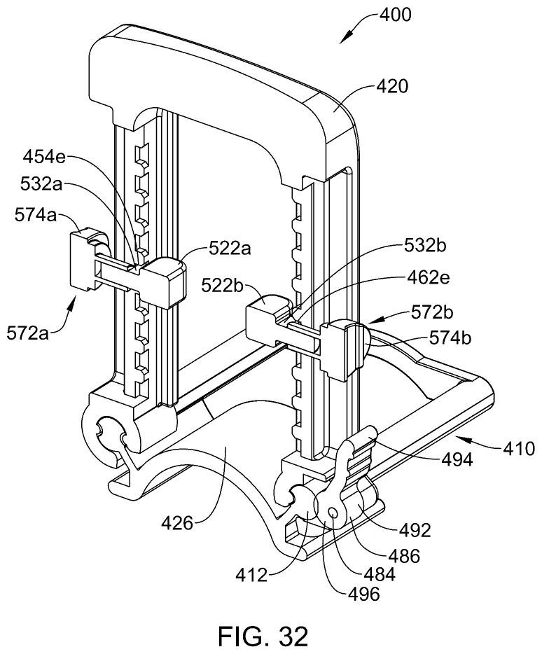

FIG. 32 is a cross-sectional view of the illustrative guide system 400 taken at line 32-32 of FIG. 30. The locking mechanism 486 may include a pivoting member 492 and an actuating member 494. The locking mechanism 486 may be movable between a locked configuration configured to prevent or limit movement of the vertical support 420 along the horizontal slide member 410 and an unlocked configuration configured to allow for movement of the vertical support 420 along the horizontal slide member 410. FIG. 32 depicts the locking mechanism 486 in the first or locked configuration. The pivoting member 492 may have a generally cylindrical shape defining a lumen 496 extending axially therethrough. The pin 484 may extend through the first and second apertures 482a, 482b and through the lumen 496 to secure the locking mechanism 486 to the vertical support 420. The lumen 496 may be eccentrically located, or off-center, such that a wall thickness of the pivoting member 492 (e.g., distance from the outer surface of the pivoting member 492 to the lumen) varies around the circumference of pivoting member 492. For example, the lumen 496 may be axial offset from a longitudinal axis of the pivoting member 492 of the locking mechanism 486.

In the locked configuration, the locking mechanism 486 is positioned such that portion of the pivoting member 492 having the thickest wall is positioned against the axially extending flattened region 425 of the second rail 412b. As the pin 484 maintains the lumen 496 in a fixed lateral location, positioning the thickest portion of the pivoting member 492 against the axially extending flattened region 425 may bias or push the second rail 412b laterally inwards against the right generally C-shaped bracket 472 to frictionally engage the protrusion 476 with the axially extending slot 422b. The frictional force between the protrusion 476 and the axially extending slot 422b may be sufficient to limit or prevent axial movement of the vertical support 420 along the horizontal slide member 410. When axial movement of the vertical support 420 is desired, the user may rotate the locking mechanism 486 via the actuating member 494. FIG. 33 a cross-sectional view of the illustrative guide system 400 with the locking mechanism 486 in a second or unlocked configuration. In the unlocked configuration, the locking mechanism 486 is positioned such that portion of the pivoting member 492 having the thinnest wall is positioned against the axially extending flattened region 425 of the second rail 412b. As the pin 484 maintains the lumen 496 in a fixed lateral location, positioning the thinnest portion of the pivoting member 492 against the axially extending flattened region 425 may remove the biasing force between the second rail 412b and the right generally C-shaped bracket 472. This may remove the frictional engagement between the protrusion 476 and the axially extending slot 422b to allow for axial movement of the vertical support 420 along the horizontal slide member 410.

In the illustrated embodiments, the actuating member 494 is generally vertically oriented in the locked configuration and generally laterally oriented in the unlocked configuration. However, this is not required. The reverse configuration is also contemplated in which the actuating member 494 is generally laterally oriented in the locked configuration and generally vertically oriented in the unlocked configuration. The guide system 400 may include visual indicia to indicate when the locking mechanism 486 is in the locked or unlocked configuration.

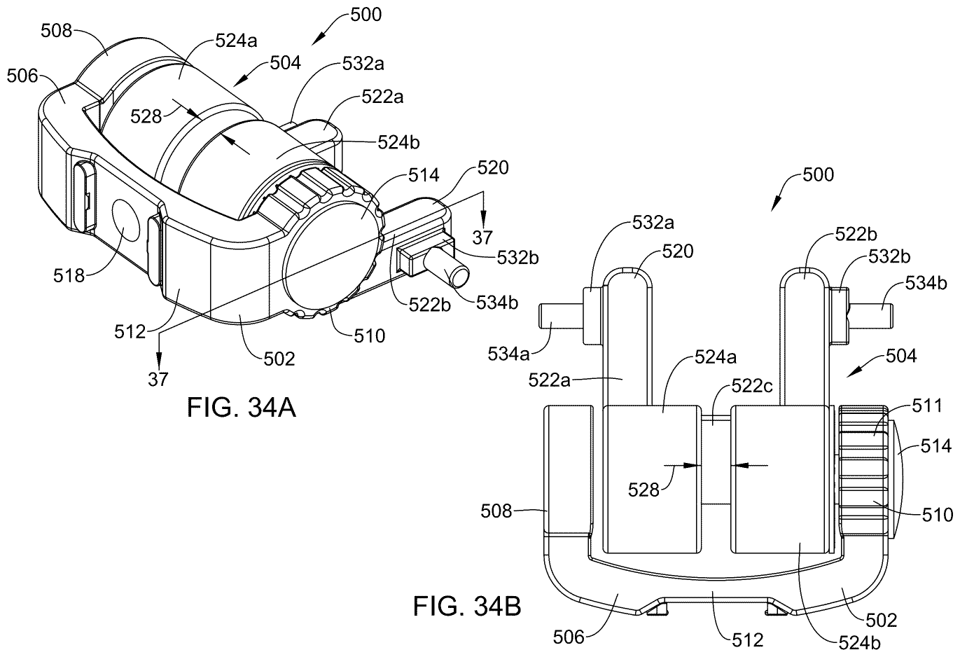

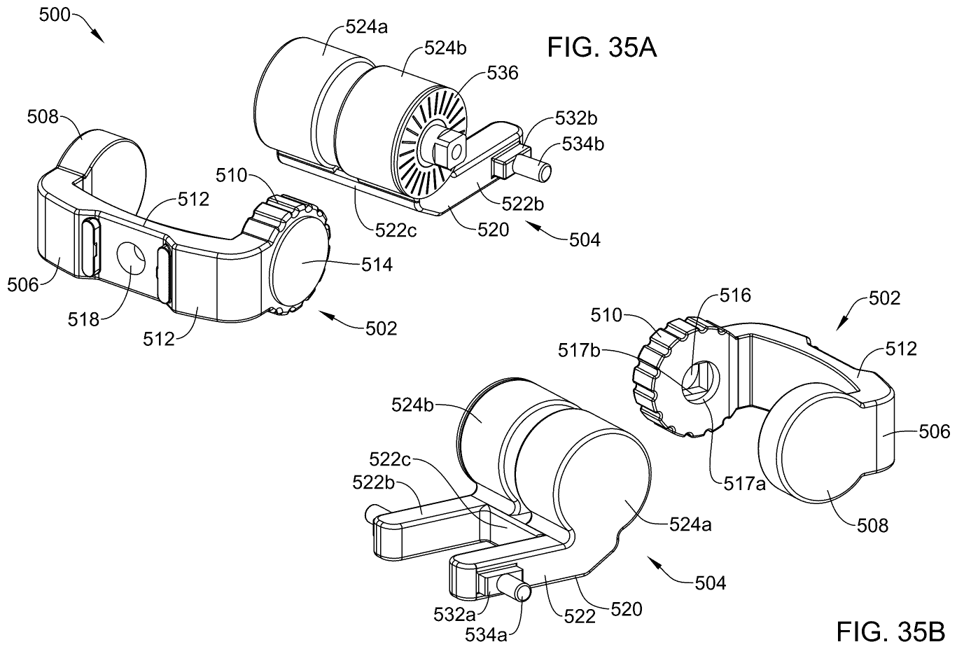

FIG. 34A is a perspective view of an illustrative needle actuation assembly 500 for use in the guide system 400. FIG. 34B is a top view of the illustrative needle actuation assembly 500. FIG. 35A is an exploded front perspective view of the needle actuation assembly 500 and FIG. 35B is an exploded back view of the needle actuation assembly 500. The needle actuation assembly 500 may include a needle mount member 502 and a gear assembly member 504. Generally, the needle actuation assembly 500 may include a first actuation system to control a vertical location of the needle actuation assembly 500 and a second actuation system to control an angular orientation of the needle mount member 502 (and thus the guide needle 172).

The needle mount member 502 may have a generally elongated U-shaped body 506 including a first or left arm 508, a second or right arm 510, and an interconnecting arm 512. The interconnecting arm 512 may have a lateral width that is greater than an axial length of the left and right arms 508, 510. The right arm 510 may include a removable cover 514 configured to enclose a laterally outward portion of a through hole or aperture 516 extending through the right arm 510. In some cases, the cover 514 may form a snap fit with the right arm 510. The through hole 516 may extend parallel to the lateral axis 404 and may be configured to receive a mating mounting member 550 of the gear assembly member 504 to selectively rotate the needle mount member 502 relative to the gear assembly member 504, as will be described in more detail herein. The interconnecting arm 512 may include an aperture 518 extending through a thickness thereof. The aperture 518 may be generally centrally located on the interconnecting arm 512. However, this is not required. The aperture 518 may be configured to receive a portion of the guide needle 172 therethrough, such as, but not limited to, the transition region 184. An outer perimeter of the right arm 510 may include grooves 511 or another surface texturing configured to increase the gripability of the right arm 510. For example, increasing the gripability of the right arm 510 may facilitate rotation of the needle mount member 502 during angular positioning of the guide needle 172.

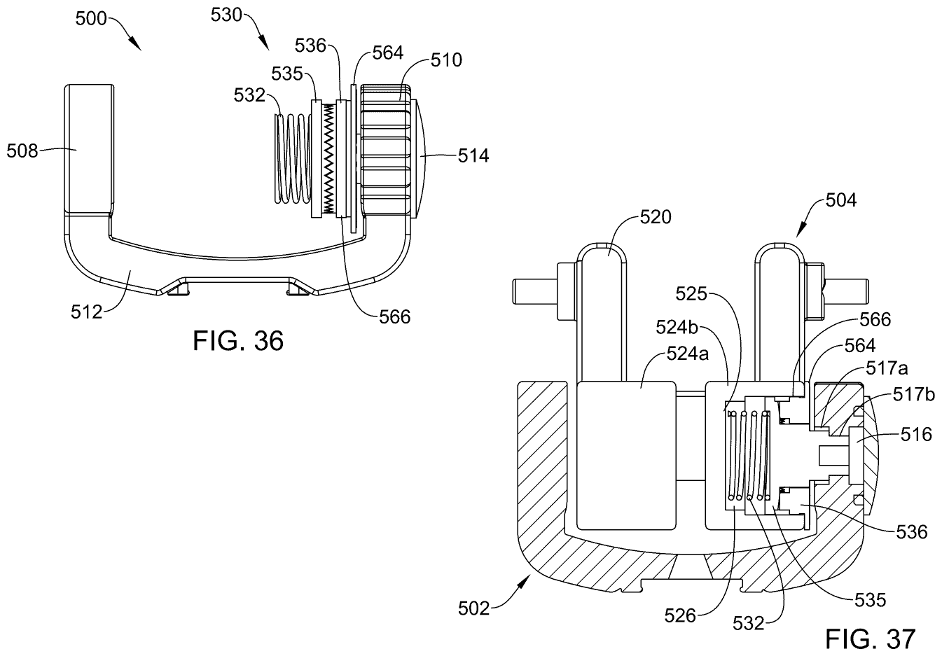

The gear assembly member 504 may include a gear assembly housing 520 including a left axially extending leg 522a, a right axially extending leg 522b extending generally parallel to the left leg 552a, and an interconnecting leg 522c extending between the left and right legs 522a, 522b. The interconnecting leg 522c may extend between front ends of the left and right legs 522a, 522b and generally orthogonal thereto. The gear assembly housing 520 may further include a first or left cylindrical member 524a and a second or right cylindrical member 524b coupled to and extending vertically from a top surface of the interconnecting leg 522c. The first cylindrical member 524a may be substantially solid. The second cylindrical member 524b may define a cavity 526 therein (see, for example, FIG. 37). For example, a laterally inward end of the second cylindrical member 524b may be substantially solid while a laterally outward end of the second cylindrical member 524b may define an opening. The cavity 526 may extend from the laterally outward end of the second cylindrical member 524b towards the laterally inward end thereof. The cavity 526 may be configured to house a gear assembly 530 (see, for example, FIGS. 36 and 37) configured to selectively couple the needle mount member 502 relative to the gear assembly member 504. The left and right cylindrical members 524a, 524b may be laterally spaced from one another by a gap 528. The gap 528 may be laterally aligned with the aperture 518 of the needle mount member 502 and sized and shaped to receive a portion of the guide needle 172 therethrough, such as, but not limited to the elongate needle region 182. For example, the gap 528 may have a lateral width that is greater than the diameter of the elongated needle portion 182 of the guide needle 172 to allow the elongated needle portion 182 to pass therethrough. The gap 528 may extend from a top portion of the left and right cylindrical members 524a, 524b to the top surface of the interconnecting leg 522c.

The gear assembly member 504 may further include a first or left protrusion 532a extending laterally outwards from a laterally outward surface of the left leg 522a and a second or right protrusion 532b extending laterally outwards from a laterally outward surface of the right leg 522b. The left and right protrusions 532a, 532b may have a generally rectangular prism shaped configured to be received within the notches 454a-i, 458a-i, 462a-i of the vertical support 420. It is contemplated that the left and right protrusions 532a, 532b may take other shapes, as desired. For example, the size and shape of the protrusions 532a, 532b may be selected to mate with the notches 454a-i, 458a-i, 462a-i. In FIG. 32, the left protrusion 532a is positioned within notch 454e and notch 458e (not explicitly shown in FIG. 32) while the right protrusion 532b is positioned within notch 462e (and the corresponding notch adjacent to the front of the right channel 452b. When the left and right protrusions 532a, 532b are positioned within the notches 454a-i, 458a-i, 462a-i, vertical movement of the needle actuation assembly 500 is precluded. As will be described in more detail herein, the left and right protrusions 532a, 532b may be moved laterally inwards to disengage the left and right protrusions 532a, 532b from the notches 454a-i, 458a-i, 462a-i to allow for vertical movement of the needle actuation assembly 500.

The gear assembly member 504 may further include a first or left mounting member 534a extending laterally outwards from a laterally outward surface of the left protrusion 532a and a second or right mounting member 534b extending laterally outwards from a laterally outward surface of the right protrusion 532b. The mounting members 534a, 534b may be configured to be received within a lumen 578a, 578b of push buttons 572a, 572b, as will be described in more detail herein. Generally, the push buttons 572a, 572b may be actuated to selectively release the protrusions 532a, 532b from the notches 454a-i, 458a-i, 462a-i.

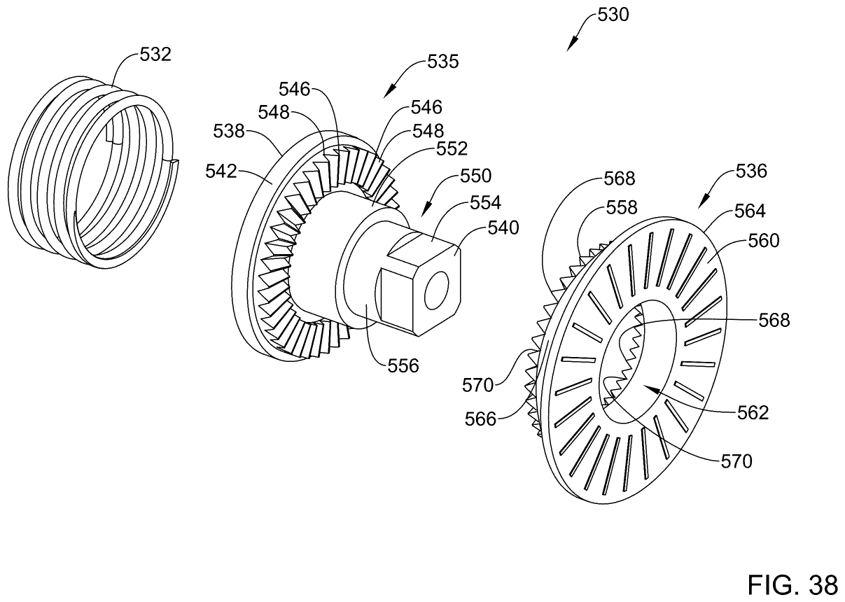

FIG. 36 is a top view of the needle actuation assembly 500 with the gear assembly housing 520 removed to more particularly illustrate the gear assembly 530. FIG. 37 is a cross-sectional view of the needle actuation assembly 500 taken at line 37-37 of FIG. 34A. The gear assembly 530 may include biasing mechanism 532, a first gear 535, and a second gear 536. The biasing mechanism 532 may be a compression spring, a wave spring washer, a leaf spring, or the like. FIG. 38 is an exploded perspective view of the gear assembly 530. The first gear 535 may extend from a first or left end 538 to a second or right end 540. The first gear 535 may include a disc member 542 at the first end 538 thereof. The disc member 542 may have a recess 544 (see, for example, FIG. 37) formed in the left end surface thereof. The recess 544 may be sized and shaped to receive an end of the biasing mechanism 532 therein. The recess 544 may extend through less than an entire thickness of the disc member 542. It is contemplated that a diameter of the recess 544 may be the same as or similar to a diameter of the biasing mechanism 532 to help prevent unintended movement of the biasing mechanism 532 relative to the first gear 535. The disc member 542 may further include a plurality of ridges 546 and valleys 548 (e.g., gear teeth) formed in or extending from a right end surface thereof, where not all of the ridges 546 and valleys 548 have been identified in the Figures for brevity and ease of understanding. The ridges 546 and valleys 548 may form an annular ring. In some embodiments, the outer diameter of the disc member 542 adjacent to the ridges 546 and valleys 548 may be less than an outer diameter at the left end 538 of the first gear 535. However, this is not required.

A stem 550 may extend laterally outward from the right end surface of the disc member 542. The stem 550 may be positioned within the annular ring of the ridges 546 and valleys 548. The stem 550 may include a first region 552 connected to the disc member 542 having a first outer diameter. The diameter of the first region 552 may be similar to a diameter of a lumen 562 of the second gear 536 to help prevent unintended movement of the stem 550 relative to the second gear 536. The stem 550 may further include a second region 554 and a third or intermediate region 556 disposed between the first region 552 and the second region 554. The second region 554 may be adjacent to the right end 540 of the first gear 535. The second region 554 may have a non-circular cross-sectional shape. For example, the cross-sectional shape of the second region 554 may be keyed, include planar regions, etc. such that rotational movement of the needle mount member 502 may be selectively translated to the first gear 535. For example, the stem 550 of the first gear 535 may be configured to extend through the second gear 536 and into the through hole 516 of the right arm 510 of the needle mount member 502. The through hole 516 may have a cross-section that changes shape to mate with the stem 550 of the first gear 535. For example, the through hole 516 may include a first region 517a having a generally circular cross-sectional shape and a second region 517b having a generally square cross-sectional shape. It is contemplated that the second region 517b may have a cross-sectional shape that mates with the second region 554 of the stem 550. The intermediate region 556 of the stem 550 may have a generally circular cross-section. However, the intermediate region 556 may take other shapes, as desired. The diameter or cross-sectional dimension of the intermediate region 556 may be less than the diameter of the first region 552. The diameter or cross-sectional dimension of the intermediate region 556 may be sized and shaped similar to the diameter or cross-section of the first region 517a of the through hole 516. In some embodiments, the cross-sectional dimension of the second region 554 may be less than the diameter or cross-sectional dimension of the intermediate region 556. In other embodiments, the cross-sectional dimension of the second region 554 may be approximately equal to the diameter or cross-sectional dimension of the intermediate region 556.

The second gear 536 may extend from a first or left end 558 to a second or right end 560. The second gear 536 may include a lumen 562 extending from the left end 538 to the right end 560. The lumen 562 may be configured to receive the stem 550 of the first gear 535 therethrough. The second gear 536 may include an annular flange 564 at the right end thereof. The annular flange 564 may have an outer diameter that is greater than an inner diameter of the cavity 526 of the second cylindrical member 524b. In some cases, the annular flange 564 may have an outer diameter that is approximately the same as the outer diameter of the second cylindrical member 524b. The laterally inward surface of the annular flange 564 may be fixed or removably secured to the right lateral surface of the second cylindrical member 524b such that rotation of the second gear 536 relative to second cylindrical member 524b (and the gear assembly housing 520) is precluded. The second gear 536 may further include an annular portion 566 extending laterally inward from the annular flange 564. The annular portion 566 may have an outer diameter that is approximately the same as the inner diameter of the cavity 526 adjacent the right end thereof to provide a friction fit. However, this is not required. The outer diameter of the annular portion 566 may be less than the inner diameter of the cavity 526 adjacent the right end thereof.

The annular portion 566 may further include a plurality of ridges 568 and valleys 570 (e.g., gear teeth) extending from a left end surface thereof, where not all of the ridges 568 and valleys 570 have been identified in the Figures for brevity and ease of understanding. The ridges 568 and valleys 570 may form an annular ring. In some embodiments, the outer diameter of the second gear 536 adjacent to the ridges 568 and valleys 570 may be less than an outer diameter at the annular portion 566. However, this is not required. The ridges 568 and valleys 570 may be configured to mate with and selectively engage the ridges 546 and valleys 548 of the first gear 535. When the 568 and valleys 570 of the second gear 536 are engaged with the ridges 546 and valleys 548 of the first gear 535 rotation of the needle mount member 502 may be precluded. For example, as the second gear 536 is rotationally fixed, the first gear 535 which is rotationally coupled to the needle mount member 502 cannot rotate when the gear teeth 546, 548, 568, 570 are engaged thus preventing swivel (angular) movement of the needle mount member 502 and the guide needle 172

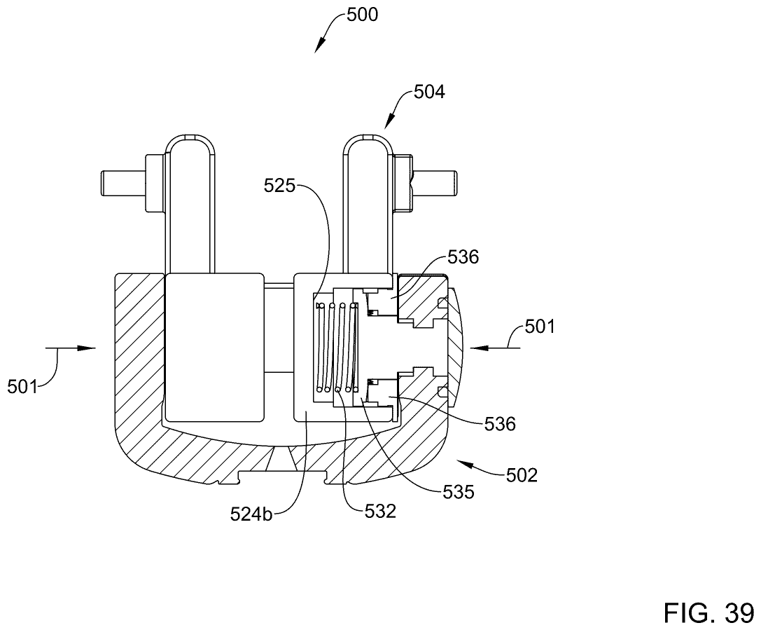

When assembled within the cavity 526 of the second cylindrical member 524b, the biasing mechanism 532 may be positioned between the interior wall 525 of the cavity 526 and the left end 538 of the first gear 535. When it is desired to pivot or angle the guide needle 172, the user may pinch or bias the left and right arms 508, 510 laterally inwards to move the needle mount member 502 and the gear assembly 530 into an unlocked configuration. FIG. 39 is a cross-sectional view of the needle actuation assembly 500 with the needle mount member 502 and the gear assembly 530 in an unlocked configuration. The arms 508, 510 have be biased laterally inwards, as shown at arrows 501. The inward force of the right arm 510 may deflect the first gear 535 laterally inwards (e.g., toward the interior wall 525 of the cavity 526). For example, the transition between the first region 517a and the second region 517b of the through hole 516 may contact the transition between the second region 554 and the intermediate region 556 of the stem 550 to transfer the lateral movement of the arm 510 to the stem 550. As the first gear 535 is moved laterally inwards, the biasing mechanism 532 compresses and the first gear 535 is moved away from the second gear 536 to disengage the ridges 546 and grooves 548 of the first gear 535 from the ridges 568 and grooves 570 of the second gear 536. With the gear teeth 546, 548 of the first gear 535 spaced from the gear teeth 568, 570 of the second gear 536, the needle mount member 502 may be rotated to adjust an angular position of the guide needle 172. This may allow the needle mount member 502 to allow the elongate needle region 182 to be angled relative to the horizontal axis 402. For example, the needle mount member 502 may be rotated to allow the elongate needle region 182 of the guide needle 172 to extend at an angle non-parallel to the horizontal axis 402. It is contemplated that the extent to which the guide needle 172 may be offset (up or down) from the horizontal axis may be determined at least in part by a vertical position of the needle actuation assembly 500 and/or the interconnecting leg 522c. When the guide needle 172 is in the desired position, the biasing force 501 on the arms 508, 510 may be removed such that the biasing mechanism 532 pushes the first gear 535 back into engagement with the second gear 536 to prevent further rotation of the needle mount member 502.

Returning to FIG. 33, the guide system 400 may further include a first or left push button 572a and a second or right push button 572b. The left and right push buttons 572a, 572b may have a same structure. The push buttons 572a, 572b may include an actuation member 574a, 574b and a stem 576a, 576b extending from the actuation member 574a, 574b. The actuation member 574a, 574b may include features configured to increase the gripability or friction thereof. For example, the actuation member 574a, 574b may include grooves, bumps, protrusions, slots, etc. along an outer surface thereof. In some cases, a lateral end surface of the actuation member 574a, 574b may include a concave recess configured to receive a user's finger. The stem 576a, 576b may include a lumen 578a, 578b extending at least partially therethrough. The lumen 578a, 578b may be configured to receive a mating mounting member 534a, 534b of the gear assembly member 504. For example, the lumens 578a, 578b may have a diameter that is greater than the outer diameter of the mounting members 534a, 534b. The diameter of the lumen 578a, 578b may be less than a maximum axial or vertical dimension of the protrusion 532a, 532b such that a free end 582a, 582b of the stem 576a, 576b contacts the laterally outward surface of the protrusion 532a, 532b.

The push buttons 572a, 572b may be assembled with the mounting members 534a, 534b such that the actuation member 574a, 576 is positioned laterally exterior to the vertical support 420 and the stems 576a, 576b extend through the first and second channels 452a, 452b of the vertical support 420. In some cases, the actuation member 574a, 574b may have an axial length that is greater than the first axial length of the first or second channels 452a, 452b at the laterally outward faces 448b, 450b thereof. This may prevent the actuation member 574a, 574b from entering the first and/or second channels 452a, 452b. However, this is not required.

In some embodiments, the push buttons 572a, 572b may include an intermediate region 580a, 580b disposed between the actuation member 574a, 574b and the stem 576a, 576b. The intermediate region 580a, 580b may have an axial length that is greater than the first axial length of the first or second channels 452a, 452b at the laterally outward faces 448b, 450b but greater than the second axial length of the first or second channels 452a, 452b at the laterally inward faces 448d, 450d. This may prevent the actuation member 574a, 574b from entering the channel 490 of the vertical support 420. However, this is not required.

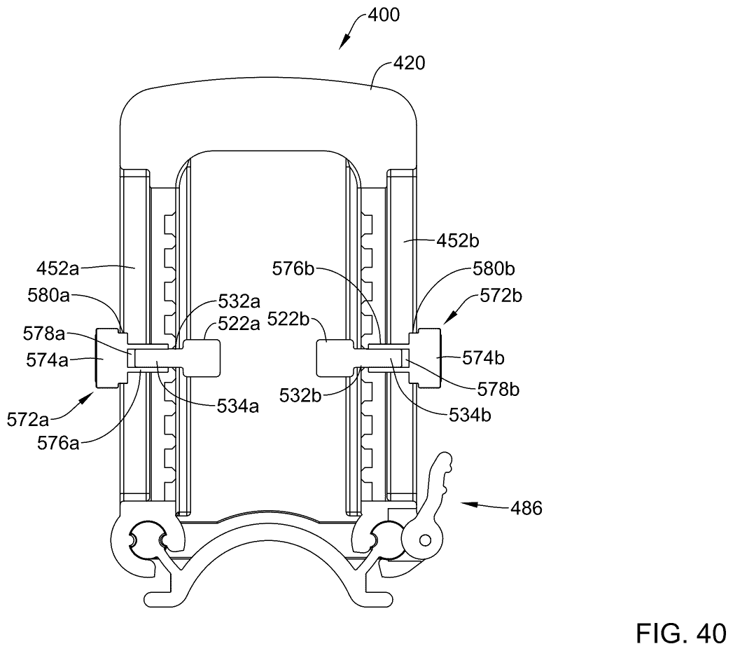

The push buttons 572a, 572b may be actuatable to temporarily disengage the protrusion 532a, 532b from the notches 454a-i, 458a-i, 462a-i of the vertical support 420. In FIG. 33, the left protrusion 532a is positioned within notches 454e, 458e (not shown in FIG. 33) and right protrusion 532b is positioned within notch 462e (and the corresponding notch at the front of the channel 452b). The positioned of the left protrusion 532a within the notches 454e, 458e may also be seen in FIG. 32. To move the needle actuation assembly 500 up and down within the channel 490 of the vertical support 420, the push buttons 572a, 572b may be pushed or biased laterally inwards into an unlocked configuration. FIG. 40 is a cross-sectional view of the guide system 400 with the push buttons 572a, 572b in an unlocked configuration. As the push buttons 572a, 572b are moved laterally inwards, the stems 576a, 576b thereof mechanically engage the protrusion 532a, 532b and bias the protrusion 532a, 532b and the left and right legs 522a, 522b of the gear assembly member 504 laterally inwards to disengage the protrusion 532a, 532b from the notches 454e, 458e, 462e (and the corresponding notch at the front of the channel 452b), as can be seen in FIG. 40. Once the protrusion 532a, 532b are free from the notches 454e, 458e, 462e (and the corresponding notch at the front of the channel 452b), the needle actuation assembly 500 and thus the guide needle 172 may be moved vertically up and/or down (e.g., top to bottom) within the vertical support 420. When the guide needle 172 is at the desired vertical position, the biasing force on the push buttons 472a, 472b may be released. The left and right legs 522a, 522b of the gear assembly member 504 may return to their original configuration and move the protrusion 532a, 532b laterally outwards into a notch 454a-i, 458a-i, 462a-i. It is contemplated that the space 460, 464 between the notches 454a-i, 458a-i, 462a-i may be curved to help guide the protrusion 532a, 532b into the nearest notch 454a-i, 458a-i, 462a-i.

In use, the guide system 400 may be secured to an ultrasound probe using a strap (although this is not required) before or after the ultrasound probe is positioned within the anatomy. For example, for a prostate biopsy, the ultrasound probe may be positioned within the rectum. Once the guide system 400 is secured to the ultrasound probe and the ultrasound probe is positioned, the vertical support 420 may be axially displaced along the horizontal slide member 410 and secured in the desired axial position. This may be performed with the locking mechanism 486 in the unlocked configurations. Next, a vertical position of the needle actuation assembly 500 (and thus a vertical position of the guide needle 172) may be adjusted. The push buttons 572a, 572b may be biased laterally inwards to allow the needle actuation assembly 500 to be moved vertically within the channel 490 of the vertical support 420. Once the needle actuation assembly 500 is at the desired height or vertical location, the push buttons 572a, 572b may be released. The arms 508, 510 of the needle mount member 502 may be biased laterally inwards and the needle mount member 502 rotated to position the guide needle 172 at a desired angle. Once the needle mount member 502 is in the desired position, the biasing force on the left and right arms 508, 510 may be released.

It is contemplated that multiple samples may be taken with the needle actuation assembly 500 at a same height. For example, the angle of the guide needle 172 may be adjusted to obtain multiple samples within a same plane. It is further contemplated that the order of the steps described herein may be rearranged. For example, the vertical and/or angular position of the guide needle 172 may be adjusted prior to moving the vertical support 420 along the horizontal slide member 410 towards or away from the patient's body.

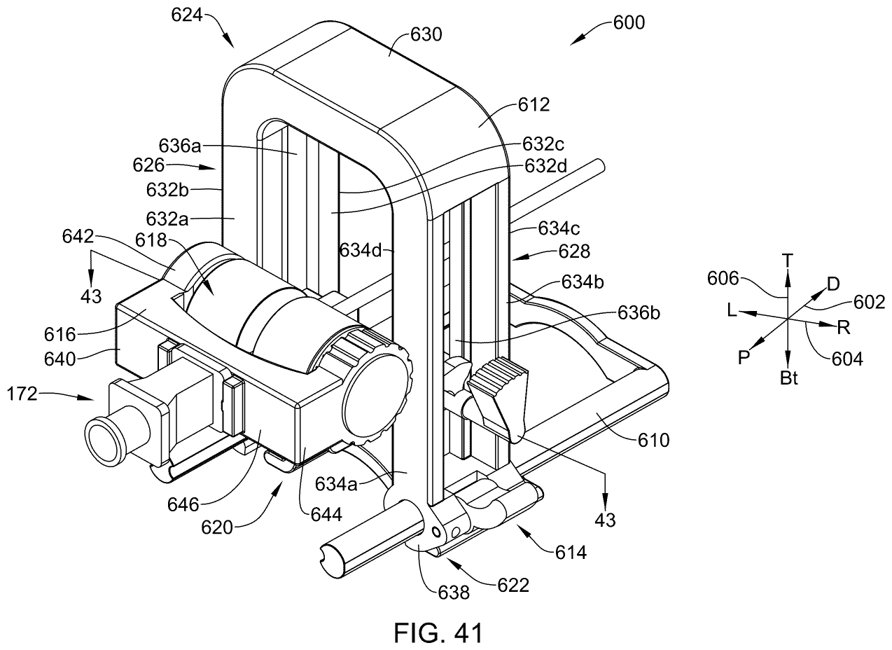

FIG. 41 is a perspective view another illustrative prostate biopsy guide system 600 in a first configuration. While the guide system 600 is described with respect to prostate biopsy, it is contemplated that the guide system 600 may be used in other procedures. In one illustrative example, the guide system 600 may be used to facilitate the injection of a spacing material, such as between the rectum and the prostate to prevent damage during radiation therapy. One illustrative system for injecting materials into the space between the rectum and the prostate is the SpaceOAR Vue™ System available from Boston Scientific Corporation, Marlborough, MA, USA. The guide system 600 may be configured to allow for horizontal, vertical, and angular movement of the biopsy needle (not explicitly shown) while also fixing the biopsy needle in a particular configuration. The guide system 600 may be configured to mount to an ultrasound probe (not explicitly shown) such that the guide system 600 is in a generally fixed position relative to the ultrasound probe. As used herein, movement in the horizontal direction or relational positioning in a direction generally parallel to axis 602, will be described as axial movement or an axial direction with relational positioning using proximal (P) (or in some cases, front) and distal (D) (or in some cases, back); movement or relational positioning in a direction generally parallel to axis 604 will be described as lateral movement or a lateral direction with relational positioning using left (L) and right (R); and movement or relational positioning in a direction generally parallel to axis 606 will be described as vertical movement or a vertical direction with relational positioning using top (T) and bottom (Bt). The use of the terms “proximal”, “distal”, “front,” “back”, “left”, “right”, “top”, “bottom”, “up”, and “down” are not intended to limit the guide system 600 to a particular orientation, but rather facilitate discussion of relative orientation.

Generally, the guide system 600 may be configured to control a horizontal position, a vertical position, and/or an angle of a guide needle. The guide system 600 may be configured to lock the guide needle 172 (as shown and described with respect to FIG. 9) in a desired orientation independent of an ultrasound probe. It is contemplated that guide needles having alternative configurations may also be used with the guide system 600. Further, the vertical and/or angular position may be held as the guide needle is moved towards the patient's body and/or away from the patient's body. The guide system 600 may include a horizontal slide member 610, a vertical support 612, a locking mechanism 614, a needle mount member 616, and a gear assembly member 618, among other features. The needle mount member 616 may be pivotable relative to the needle mount member 616 to change an angle of the guide needle 172. Further, the needle actuation assembly 620 may be configured to move up and down along the vertical support 612 to control a vertical position of the needle mount member 616 (and thus the guide needle 172). The guide system 600 may include locking features to selectively lock a vertical position of the needle actuation assembly 620 and an angular position of the needle mount member 616. The vertical support 612 may be moved along the horizontal slide member 610 to move the guide needle 172 distally towards and/or proximally away from the patient. The vertical support 612 may be selectively secured to the horizontal slide member 610 to lock the vertical support 612 in a desired axial position.

The horizontal slide member 610 may be similar in form and function to the horizontal slide member 410 described with respect to FIGS. 30-40. For example, the horizontal slide member 610 includes parallel rails for mating with a portion of the vertical support 612, a probe mount, and the like. The parallel rails include features configured to mate with the vertical support 612, as well as features to facilitate horizontal locking of the vertical support 612, as described above. For brevity, the structure of the horizontal slide member 610 will not be repeated but should be considered to include all of the structure features of the horizontal slide member 410 described herein.

The vertical support 612 may be configured to be axially displaced along the horizontal slide member 610. The vertical support 612 may be similar in form and function to the vertical support 420 described herein. However, the guide system 600 may include an alternative vertical locking system which does not require the notches 454a-i, 458a-i, 462a-i shown and described with respect to FIGS. 30-40. Generally, the vertical support 612 may extend from a bottom end region 622 to a top end region 624. The vertical support 612 may be formed as a single monolithic structure or as two or more components coupled together. The vertical support 612 may have a generally inverted “U” shape having a first or left arm 626 a second or right arm 628 laterally spaced from the left arm 626, and an interconnecting arm 630 extending between a top region 624 of the left arm 626 and the right arm 628

The left arm 626 and the right arm 628 may extend generally parallel to one another. The interconnecting arm 630 may extend generally orthogonal to the left arm 626 and the right arm 628. The left arm 626 may include a proximal face 632a, a left (or laterally outward face) 632b, a distal face 632c, and a right (or laterally inward face) 632d. The right arm 628 may include a proximal face 634a, a right (or laterally outward face) 634b, a distal face 634c, and a left (or laterally inward face) 634d.

The left arm 626 may include a first channel 636a extending from the laterally outward face 632b to the laterally inward face 632d. The first channel 636a may have a first axial length at the laterally outward face 632b and a second axial length at the laterally inward face 632d. The second axial length may be less than the first axial length. In some examples, a height of the first channel 636a may be greater at the laterally outward face 632b than a height of the first channel 636a at the laterally inward face 632d. The right arm 628 may include a second channel 636b extending from the laterally outward face 634b to the laterally inward face 634d. The second channel 636b may have a first axial length at the laterally outward face 634b and a second axial length at the laterally inward face 634d. The second axial length may be less than the first axial length. In some examples, a height of the second channel 636b may be greater at the laterally outward face 634b than a height of the second channel 636b at the laterally inward face 634d.

The left arm 626 may further include a first or left generally C-shaped bracket (not explicitly shown) positioned along a bottom region 622 thereof. Similarly, the right arm 628 may include a second or right generally C-shaped bracket 638. The left and right brackets 638 may be similar in form and function to the left and right generally C-shaped brackets 466, 472 described with respect to FIGS. 30-40. For example, the brackets 638 may include features configured to engage the horizontal slide member 610. Further, the right generally C-shaped bracket 638 may include features configured to engage the locking mechanism 614. In some cases, the features configured to engage the locking mechanism 614 may be provided additionally or alternatively at the left generally C-shaped bracket. For brevity, the structure of the backets 638 will not be repeated but should be considered to include all of the structure features of the brackets 466, 472 described herein.

The locking mechanism 614 may be similar in form and function to the locking mechanism 486 described with respect to FIGS. 30-40. For example, the locking mechanism 614 may be movable between a locked configuration configured to prevent or limit movement of the vertical support 612 along the horizontal slide member 610 and an unlocked configuration configured to allow for movement of the vertical support 612 along the horizontal slide member 610. For brevity, the structure of the locking mechanism 614 will not be repeated but should be considered to include all of the structure features of locking mechanism 486 described herein.

The guide system 600 may further include a needle actuation assembly 620 for use in the guide system 600. The needle actuation assembly 620 may include a needle mount member 616 and a gear assembly member 618. Generally, the needle actuation assembly 620 may include a first actuation system to control a vertical location of the needle actuation assembly 620 and a second actuation system to control an angular orientation of the needle mount member 616 (and thus the guide needle 172). The angular orientation of the needle mount member 616 (and thus the guide needle 172) may function in a similar manner to that described above with respect to FIGS. 30-40.

The needle mount member 616 may be similar in form and function to the needle mount member 502 described with respect to FIGS. 30-40. For example, the needle mount member 616 may have a generally elongated U-shaped body 640 including a first or left arm 642, a second or right arm 644, and an interconnecting arm 646. The left and right arms 642, 644 may be moved laterally inwards to engage a gear assembly within the gear assembly member 618 in a manner similar to that described above. For brevity, the structure of the needle mount member 616 will not be repeated but should be considered to include all of the structure features of the needle mount member 502 described herein.

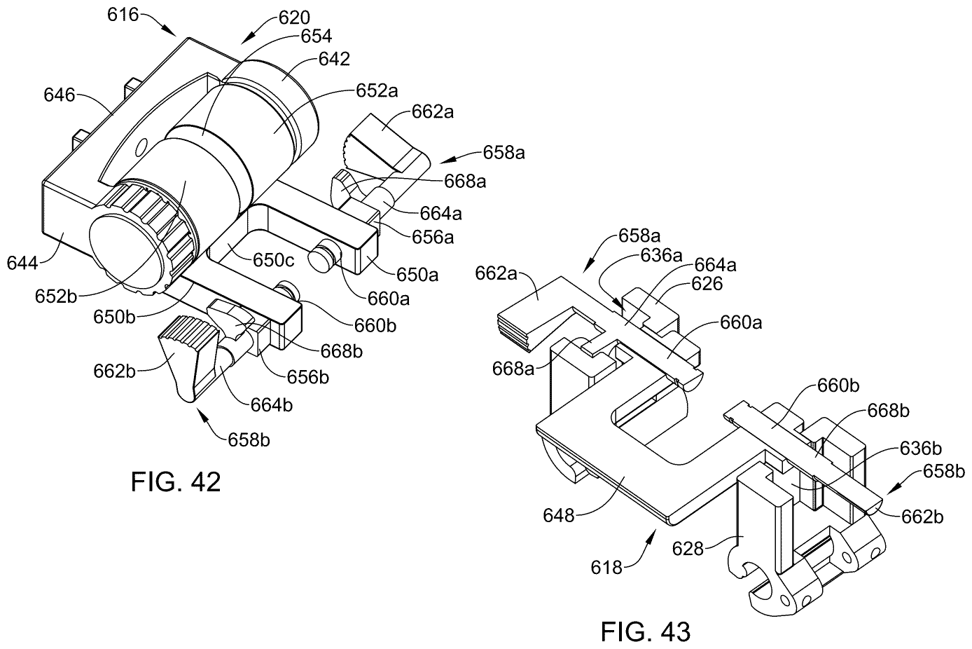

Referring additionally to FIG. 42, which is a rear perspective view of the illustrative needle actuation assembly 620, the gear assembly member 618 may include a gear assembly housing 648 including a left axially extending leg 650a, a right axially extending leg 650b extending generally parallel to the left leg 552a, and an interconnecting leg 650c extending between the left and right legs 650a, 650b. The interconnecting leg 650c may extend between front ends of the left and right legs 650a, 650b and generally orthogonal thereto. The gear assembly housing 648 may further include a first or left cylindrical member 652a and a second or right cylindrical member 652b coupled to and extending vertically from a top surface of the interconnecting leg 650c. The first cylindrical member 652a may be substantially solid. The second cylindrical member 652b may define a cavity therein (not explicitly shown), similar in form and function to the cavity 526 described with respect to FIGS. 30-40. The cavity may be configured to house a gear assembly (not explicitly shown) similar in form and function to gear assembly 530 described with respect to FIGS. 30-40. The gear assembly may be configured to selectively couple the needle mount member 616 relative to the gear assembly member 618. The left and right cylindrical members 652a, 652b may be laterally spaced from one another by a gap 654. The gap 654 may be laterally aligned with an aperture of the needle mount member 616 and sized and shaped to receive a portion of the guide needle 172 therethrough, such as, but not limited to the elongate needle region 182. For example, the gap 654 may have a lateral width that is greater than the diameter of the elongated needle portion 182 of the guide needle 172 to allow the elongated needle portion 182 to pass therethrough. The gap 654 may extend from a top portion of the left and right cylindrical members 652a, 652b to the top surface of the interconnecting leg 650c.

The gear assembly member 618 may further include a first or left protrusion 656a extending laterally outwards from a laterally outward surface of the left leg 650a and a second or right protrusion 656b extending laterally outwards from a laterally outward surface of the right leg 650b. The left and right protrusions 656a, 656b may have a generally rectangular prism shaped configured to be received within the left and right channels 636a, 636b of the vertical support 612. It is contemplated that the left and right protrusions 656a, 656b may take other shapes, as desired.

The gear assembly member 618 may further include a first or left actuation member 658a extending through an aperture 660a in the left arm 650a of the gear assembly housing 648 and a second or right actuation member 658b extending through an aperture 660b in the right arm 650b of the gear assembly housing 648. The actuation members 658a, 658b may releasably couple the needle actuation assembly 620 to the vertical support 612 as well as be actuatable to selectively lock a vertical position of the needle actuation assembly 620 relative to the vertical support 612. The left actuation member 658a may include a gripping member or knob 662a at a laterally outward end thereof. The knob 662a may be coupled with or formed as a single monolithic structure with an elongate shaft 664a. The elongate shaft 664a may be configured to extend through the aperture 660a in the left arm 650a of the gear assembly housing 648. The elongate shaft 664a may form a friction fit with the aperture 660a or may be configured to receive a nut, clamp, or other securement member (not explicitly shown) at a laterally inward end thereof to secure the elongate shaft 664a relative to the needle actuation assembly 620. The left actuation member 658a may further include a cam member 668a extending radially from the elongate shaft 664a. The cam member 668a may rotate with the elongate shaft 664a to move from a locked configuration to an unlocked configuration, as will be described in more detail herein.

The right actuation member 658b may include a gripping member or knob 662b at a laterally outward end thereof. The knob 662b may be coupled with or formed as a single monolithic structure with an elongate shaft 664b. The elongate shaft 664b may be configured to extend through the aperture 660b in the right arm 650b of the gear assembly housing 648. The elongate shaft 664b may form a friction fit with the aperture 660a or may be configured to receive a nut, clamp, or other securement member (not explicitly shown) at a laterally inward end thereof to secure the elongate shaft 664b relative to the needle actuation assembly 620. The right actuation member 658b may further include a cam member 668b extending radially from the elongate shaft 664b. The cam member 668b may rotate with the elongate shaft 664b to move from a locked configuration to an unlocked configuration, as will be described in more detail herein.

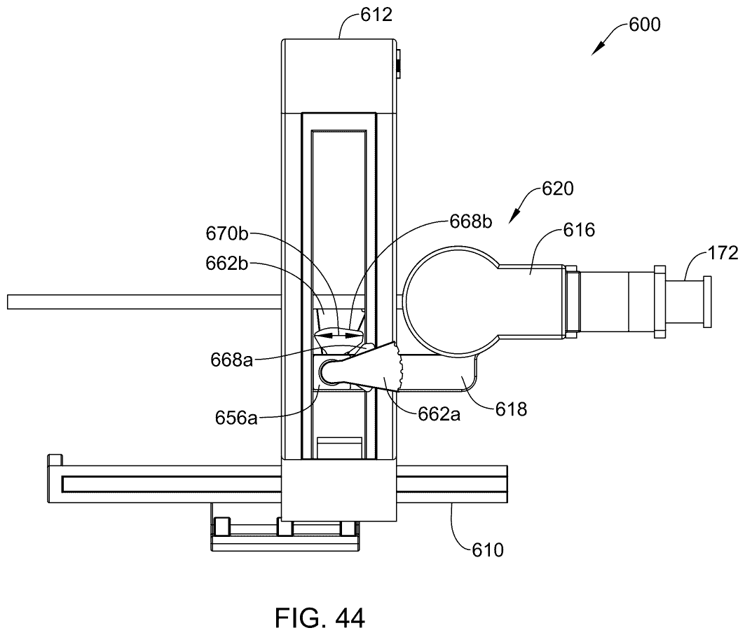

FIG. 43 is a cross-sectional view of the guide system 600, taken at line 43-43 of FIG. 41 with the horizontal slide member 610, locking mechanism 614, and guide needle 172 not shown for ease of understanding. FIG. 44 is a left side view of the guide system 600. The left and right actuation members 658a, 658b may be individually actuatable. In FIGS. 41-44, the left actuation member 658a is illustrated in a locked configuration while the right actuation member 658b is in an unlocked configuration. When the needle actuation assembly 620 is assembled with the vertical support 612, knobs 662a, 662b may be positioned laterally outside of the of the left and right arms 626, 628 of the vertical support 612 such that the user may grip and rotate the knobs 662a, 626b. The elongate shafts 664a, 664b may extend from the knobs 662a, 662b through the channels 632a, 632b and through apertures 660a, 660b of the gear assembly housing 648 of the gear assembly member 618 laterally inwards of the arms 626, 628. The cam members 668a, 668b may be positioned within the left and right channels 632a, 632b, respectively.

When the actuation member 658a is in the locked configuration, the cam member 668a extends in an axial direction to contact and engages a proximal (front) interior surface of the left channel 632a. As the knob 662a is actuated to move the cam member 668a to the locked configuration, the protrusion 656a engages a distal (back) interior surface of the left channel 632a such that the protrusion 656a and/or the cam member 668a form a mechanical or friction engagement with the vertical support 612 to prevent vertical (up and/or down) movement of the needle actuation assembly 620. While not explicitly shown, the right actuation member 658b may be rotated to move the right cam member 668b into engagement with the proximal (front) interior surface of the right channel 632b and the protrusion 656b engages a distal (back) interior surface of the right channel 632b.

When the actuation member 658b is in the unlocked configuration, the cam member 668b extends in a vertical direction. The cam member 668b may have a length 670b (see, for example, FIG. 44) that is less than the smallest axial length of the channel 632b. This may allow the cam member 668b to move freely within channel 632b when the actuation member 658b is in the unlocked configuration. It is contemplated that to move the needle actuation assembly 620 vertically relative to the vertical support 612, both the left and right actuation members 658a, 658b are actuated to the unlocked configuration. The needle actuation assembly 620 may then be moved up and/or down within the vertical support 612. Once the needle actuation assembly 620 is in the desired vertical location, the left and right actuation members 658a, 658b are actuated to the locked configuration.

In the illustrated embodiments, the knobs 662a, 662b are generally vertically oriented in the unlocked configuration and generally laterally oriented in the locked configuration. However, this is not required. The reverse configuration is also contemplated in which the knobs 662a, 662b are generally laterally oriented in the unlocked configuration and generally vertically oriented in the locked configuration. The guide system 600 may include visual indicia to indicate when the actuating members 658a, 658b are in the locked or unlocked configuration.

In use, the guide system 600 may be secured to an ultrasound probe using a strap (although this is not required) before or after the ultrasound probe is positioned within the anatomy. For example, for a prostate biopsy, the ultrasound probe may be positioned within the rectum. Once the guide system 600 is secured to the ultrasound probe and the ultrasound probe is positioned, the vertical support 612 may be axially displaced along the horizontal slide member 610 and secured in the desired axial position. This may be performed with the locking mechanism 614 in the unlocked configurations. Next, a vertical position of the needle actuation assembly 620 (and thus a vertical position of the guide needle 172) may be adjusted. The actuation members 658a, 658b may be rotated to an unlocked configuration via the knobs 662a, 662b to allow the needle actuation assembly 620 to be moved vertically within the channel (similar in form and function to channel 490) of the vertical support 612. Once the needle actuation assembly 620 is at the desired height or vertical location, actuation members 658a, 658b may be rotated to a locked configuration via the knobs 662a, 662b. The arms 642, 644 of the needle mount member 616 may be biased laterally inwards and the needle mount member 616 rotated to position the guide needle 172 at a desired angle, as described with respect to FIGS. 30-40. Once the needle mount member 616 is in the desired position, the biasing force on the left and right arms 642, 644 may be released.

It is contemplated that multiple samples may be taken with the needle actuation assembly 620 at a same height. For example, the angle of the guide needle 172 may be adjusted to obtain multiple samples within a same plane. It is further contemplated that the order of the steps described herein may be rearranged. For example, the vertical and/or angular position of the guide needle 172 may be adjusted prior to moving the vertical support 612 along the horizontal slide member 610 towards or away from the patient's body.

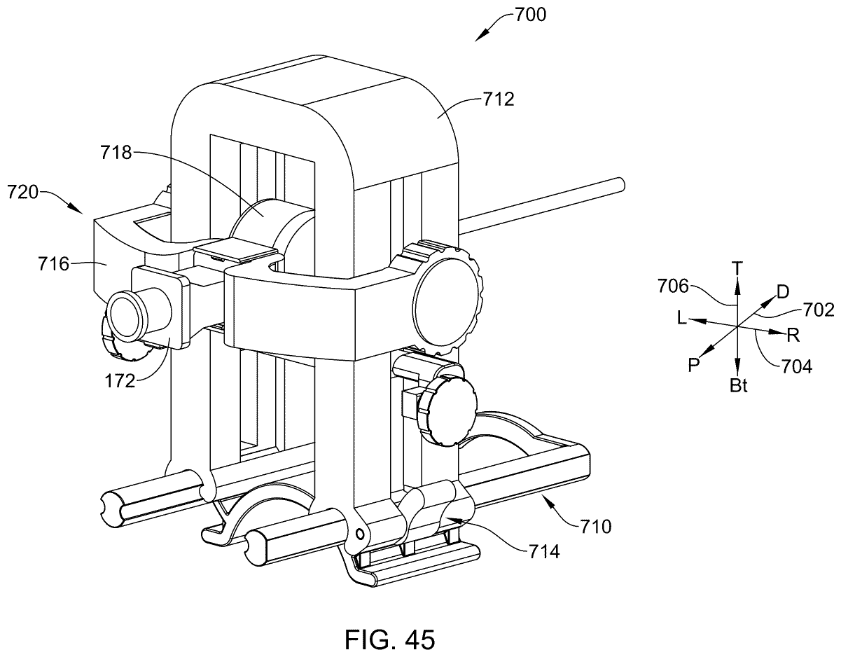

FIG. 45 is a perspective view another illustrative prostate biopsy guide system 700 in a first configuration. While the guide system 700 is described with respect to prostate biopsy, it is contemplated that the guide system 700 may be used in other procedures. In one illustrative example, the guide system 700 may be used to facilitate the injection of a spacing material, such as between the rectum and the prostate to prevent damage during radiation therapy. One illustrative system for injecting materials into the space between the rectum and the prostate is the SpaceOAR Vue™ System available from Boston Scientific Corporation, Marlborough, MA, USA. The guide system 700 may be configured to allow for horizontal, vertical, and angular movement of the biopsy needle (not explicitly shown) while also fixing the biopsy needle in a particular configuration. The guide system 700 may be configured to mount to an ultrasound probe (not explicitly shown) such that the guide system 700 is in a generally fixed position relative to the ultrasound probe. As used herein, movement in the horizontal direction or relational positioning in a direction generally parallel to axis 702, will be described as axial movement or an axial direction with relational positioning using proximal (P) or front and distal (D) or back; movement or relational positioning in a direction generally parallel to axis 704 will be described as lateral movement or a lateral direction with relational positioning using left (L) and right (R); and movement or relational positioning in a direction generally parallel to axis 706 will be described as vertical movement or a vertical direction with relational positioning using top (T) and bottom (Bt). The use of the terms ““proximal”, “distal”, “front,” “back”, “left”, “right”, “top”, “bottom”, “up”, and “down” are not intended to limit the guide system 700 to a particular orientation, but rather facilitate discussion of relative orientation.

Generally, the guide system 700 may be configured to control a horizontal position, a vertical position, and/or an angle of a guide needle. The guide system 700 may be configured to lock the guide needle 172 (as shown and described with respect to FIG. 9) in a desired orientation independent of an ultrasound probe. It is contemplated that guide needles having alternative configurations may also be used with the guide system 700. Further, the vertical and/or angular position may be held as the guide needle is moved towards the patient's body and/or away from the patient's body. The guide system 700 may include a horizontal slide member 710, a vertical support 712, a locking mechanism 714, a needle mount member 716, and a gear assembly member 718, among other features. The needle mount member 716 may be pivotable relative to the gear assembly member 718 to change an angle of the guide needle 172. Further, the needle actuation assembly 720 may be configured to move up and down along the vertical support 712 to control a vertical position of the needle mount member 716 (and thus the guide needle 172). The guide system 700 may include locking features to selectively lock a vertical position of the needle actuation assembly 720 and an angular position of the needle mount member 716. The vertical support 712 may be moved along the horizontal slide member 710 to move the guide needle 172 distally towards and/or proximally away from the patient. The vertical support 712 may be selectively secured to the horizontal slide member 710 to lock the vertical support 712 in a desired axial position.

The horizontal slide member 710 may be similar in form and function to the horizontal slide member 410 described with respect to FIGS. 30-40. For example, the horizontal slide member 710 includes parallel rails for mating with a portion of the vertical support 712, a probe mount, and the like. The parallel rails include features configured to mate with the vertical support 712, as well as features to facilitate horizontal locking of the vertical support 712, as described above. For brevity, the structure of the horizontal slide member 710 will not be repeated but should be considered to include all of the structure features of the horizontal slide member 410 described herein.

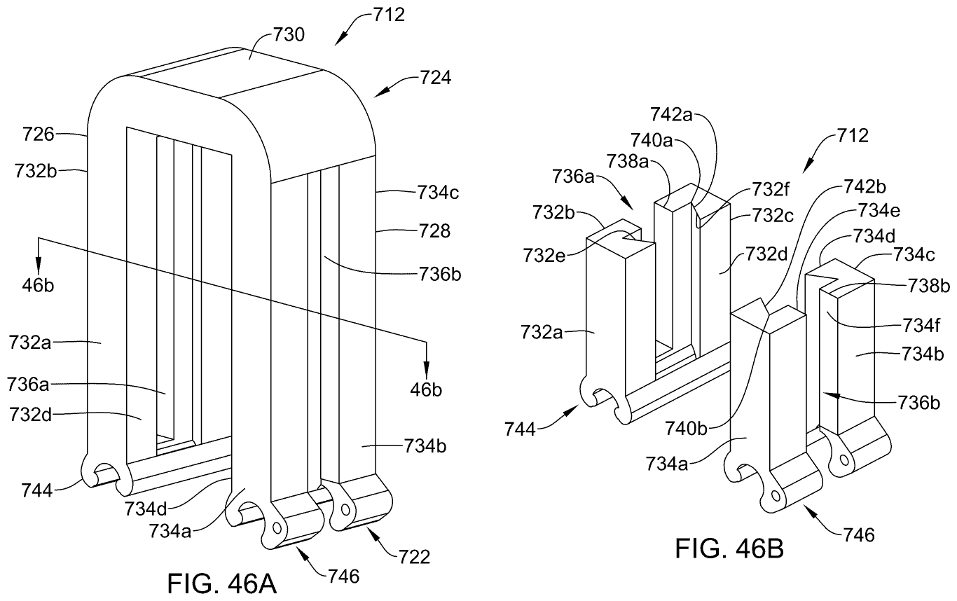

The vertical support 712 may be configured to be axially displaced along the horizontal slide member 710. FIG. 46A is a perspective view of an illustrative vertical support 712 and FIG. 46B partial cross-sectional view of the illustrative vertical support 712 taken at line 46B-46B of FIG. 46A. The vertical support 712 may extend from a bottom end region 722 to a top end region 724. The vertical support 712 may be formed as a single monolithic structure or as two or more components coupled together. The vertical support 712 may have a generally inverted “U” shape having a first or left arm 726 a second or right arm 728 laterally spaced from the left arm 726, and an interconnecting arm 730 extending between a top region 724 of the left arm 726 and the right arm 728.

The left arm 726 and the right arm 728 may extend generally parallel to one another. The interconnecting arm 730 may extend generally orthogonal to the left arm 726 and the right arm 728. The left arm 726 may include a proximal face 732a, a left (or laterally outward face) 732b, a distal face 732c, and a right (or laterally inward face) 732d. The right arm 728 may include a proximal face 734a, a right (or laterally outward face) 734b, a distal face 734c, and a left (or laterally inward face) 734d.

The left arm 726 may include a first or left channel 736a extending from the laterally outward face 732b to the laterally inward face 732d. The first channel 736a may include a first region 738a having a generally constant axially length. The first region 738a may extend laterally inwards from the laterally outward face 732b to an intermediate location 740a between the laterally outward face 732b and the laterally inward face 732d. The axial length of the first channel 736a may increase from the first axial length to a second axial length in an abrupt or stair-step manner at the intermediate location 740a. The first channel 736a may include a second region 742a extending laterally inwards from the intermediate location 740a to the laterally inward face 732d. The axial length of the second region 742a may taper or gradually reduce in length to a third axial length at the laterally inward face 732d that is less than the second axial length and greater than the first axial length. The proximal face 732e of the first channel 736a and the distal face 732f of the first channel 736a may each taper at a same angle along the second region 742a of the first channel 736a. In some cases, the cross-sectional shape of the first region 738a of the first channel 736a may be rectangular while the cross-sectional shape of the second region 742a of the first channel 736a may be an isosceles trapezoid. However, the first region 738a and/or second region 742a may take other cross-sectional shapes, as desired.

The right arm 728 may include a second or right channel 736b extending from the laterally outward face 734b to the laterally inward face 734d. The second channel 736b may include a first region 738b having a generally constant axially length. The first region 738b may extend laterally inwards from the laterally outward face 734b to an intermediate location 740b between the laterally outward face 734b and the laterally inward face 734d. The axial length of the second channel 736b may increase from the first axial length to a second axial length in an abrupt or stair-step manner at the intermediate location 740b. The second channel 736b may include a second region 742b extending laterally inwards from the intermediate location 740b to the laterally inward face 734d. The axial length of the second region 742b may taper or gradually reduce in length to a third axial length at the laterally inward face 734d that is less than the second axial length and greater than the first axial length. The proximal face 734e of the second channel 736b and the distal face 734f of the second channel 736b may each taper at a same angle along the second region 742b of the second channel 736b. In some cases, the cross-sectional shape of the first region 738b of the second channel 736b may be rectangular while the cross-sectional shape of the second region 742b of the second channel 736b may be an isosceles trapezoid. However, the first region 738b and/or second region 742b may take other cross-sectional shapes, as desired.

The left arm 726 may further include a first or left generally C-shaped (or truncated C-shaped) bracket 744 positioned along a bottom region 722 thereof. Similarly, the right arm 728 may include a second or right generally C-shaped bracket 746. The left and right brackets 744, 746 may be similar in form and function to the left and right generally C-shaped brackets 466, 472 described with respect to FIGS. 30-40. For example, the brackets 744, 746 may include features configured to engage the horizontal slide member 710. Further, the right generally C-shaped bracket 746 may include features configured to engage the locking mechanism 714. In some cases, the features configured to engage the locking mechanism 714 may be provided additionally or alternatively at the left generally C-shaped bracket. For brevity, the structure of the backets 746 will not be repeated but should be considered to include all of the structure features of the brackets 466, 472 described herein.

The locking mechanism 714 may be similar in form and function to the locking mechanism 486 described with respect to FIGS. 30-40. For example, the locking mechanism 714 may be movable between a locked configuration configured to prevent or limit movement of the vertical support 612 along the horizontal slide member 710 and an unlocked configuration configured to allow for movement of the vertical support 712 along the horizontal slide member 710. For brevity, the structure of the locking mechanism 714 will not be repeated but should be considered to include all of the structure features of locking mechanism 486 described herein.

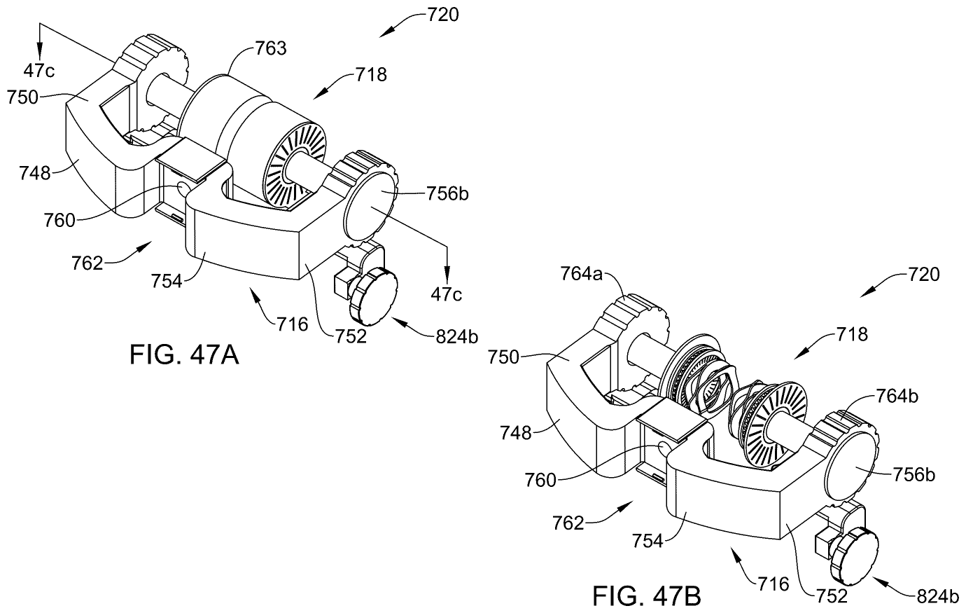

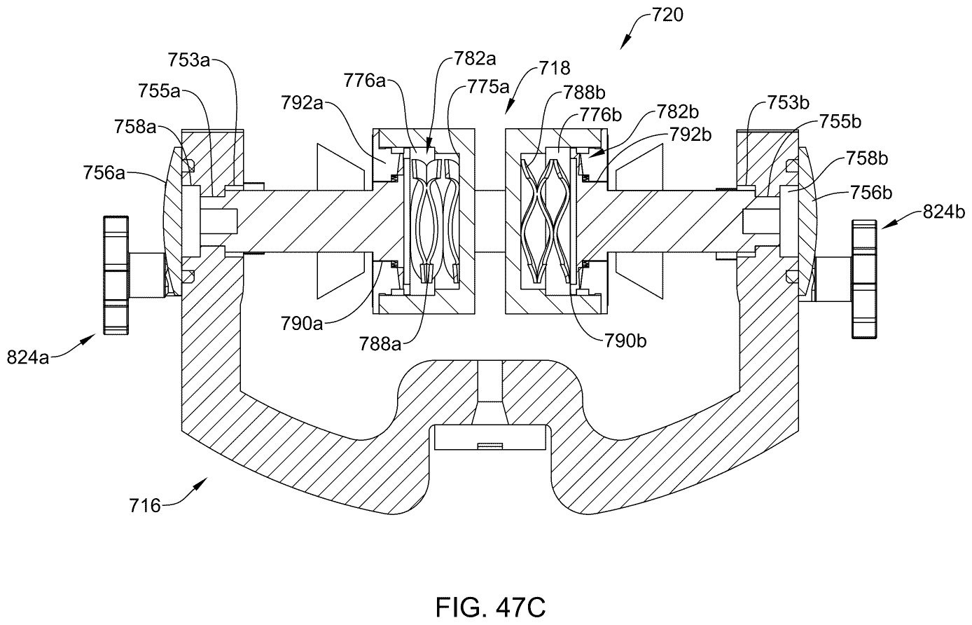

FIG. 47A is a perspective view of an illustrative needle actuation assembly 720 for use in the guide system 700. FIG. 47B is a perspective view of the illustrative needle actuation assembly 720 with a housing 763 of the gear assembly member 718 removed. FIG. 47C is a cross-sectional view of the needle actuation assembly 720, taken at line 47C-47C of FIG. 47A. The needle actuation assembly 720 may include a needle mount member 716 and a gear assembly member 718. Generally, the needle actuation assembly 720 may include a first actuation system to control a vertical location of the needle actuation assembly 720 and a second actuation system to control an angular orientation of the needle mount member 716 (and thus the guide needle 172).

The needle mount member 716 may have a generally elongated U-shaped body 748 including a first or left arm 750, a second or right arm 752, and an interconnecting arm 754. The interconnecting arm 754 may have a lateral width that is greater than an axial length of the left and right arms 750, 752. The left and right arms 750, 752 may each include a removable cover 756a, 756b configured to enclose a laterally outward portion of a through hole or aperture 758a, 758b extending through the left and right arms 750, 752, respectively. In some cases, the covers 756a, 756b may form a snap fit with the left and right arms 752. The through hole 758a, 758b may extend parallel to the lateral axis 704 and may be configured to receive a mating mounting member 786a, 786b of the gear assembly member 718 to selectively rotate the needle mount member 716 relative to the gear assembly member 718, as will be described in more detail herein. The interconnecting arm 754 may include an aperture 760 extending through a thickness thereof. The aperture 760 may be generally centrally located on the interconnecting arm 754. However, this is not required. In some cases, the aperture 760 may be positioned within a bent or recessed portion 762 of the interconnecting arm 754. The aperture 760 may be configured to receive a portion of the guide needle 172 therethrough, such as, but not limited to, the transition region 184. An outer perimeter of the left and right arms 750, 752 may include grooves 764a, 764b or another surface texturing configured to increase the gripability of the right arm 752. For example, increasing the gripability of the left and/or right arms 750, 752 may facilitate rotation of the needle mount member 716 during angular positioning of the guide needle 172.

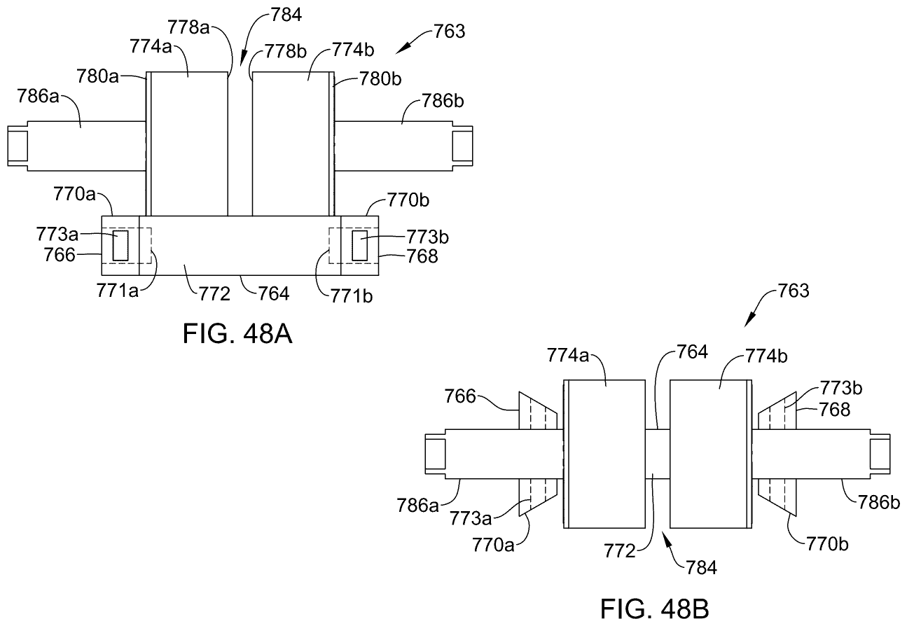

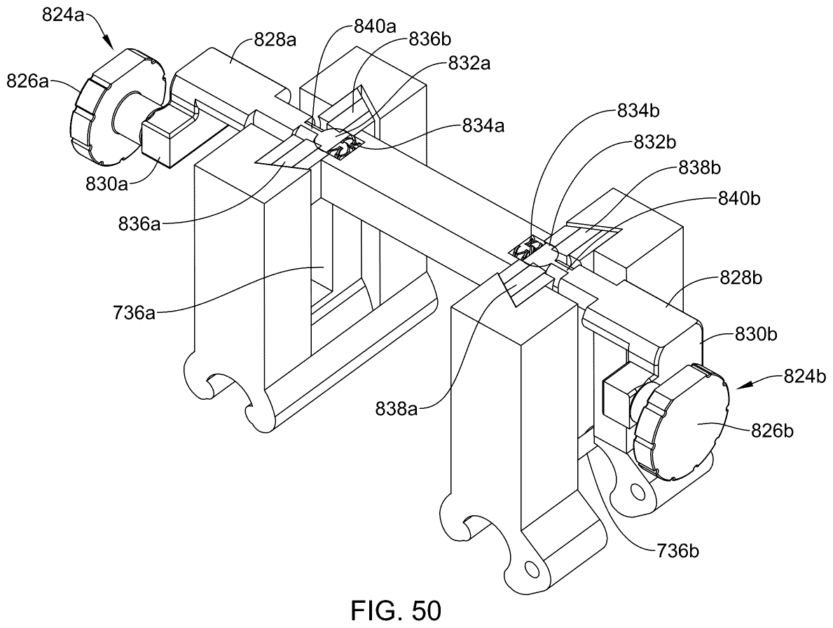

Referring additionally to FIG. 48A which is a proximal or front view of the gear assembly member 718 and FIG. 48B which is a top view of the gear assembly member 718, the gear assembly member 718 may include a gear assembly housing 763 including a base 764 extending from a left lateral side 766 to a right lateral side 768. The base 764 may include a first or left leg 770a positioned adjacent to the left lateral side 766 and a second or right leg 770b positioned adjacent to the right lateral side 768. A central beam 772 may extend laterally between the left leg 770a and the right leg 770b. The left leg 770a may have a cross section sized and shaped to move vertically within the second region 742a of the first channel 736a. For example, the cross-sectional shape of the left leg 770a may be an isosceles trapezoid. Similarly, the right leg 770b may have a cross section sized and shaped to move vertically within the second region 742b of the second channel 736b. For example, the cross-sectional shape of the right leg 770b may be an isosceles trapezoid. A left laterally inwardly extending aperture 771a may extend from the left lateral side 766 through the left leg 770a and into the central beam 772. A right laterally inward extending apertures 771b may extend from the right lateral side 768 through the right leg 770b and into the central beam 772. The left and right laterally inward extending apertures 771a, 771b may be configured to receive a portion of the actuation assemblies 824a, 824b. A left axially extending channel 773a may extend proximally to distally through the left leg 770a and a right axially extending channels 773b may extend proximally to distally through the right leg 770b. The axially extending channels 773a, 773b may each be configured to receive a pair of brake members 836a, 836b, 838a, 838b (see, for example, FIG. 50). Generally, the brake members 836a, 836b, 838a, 838b may be axially displaceable to selectively lock a vertical position of the needle actuation assembly 720.

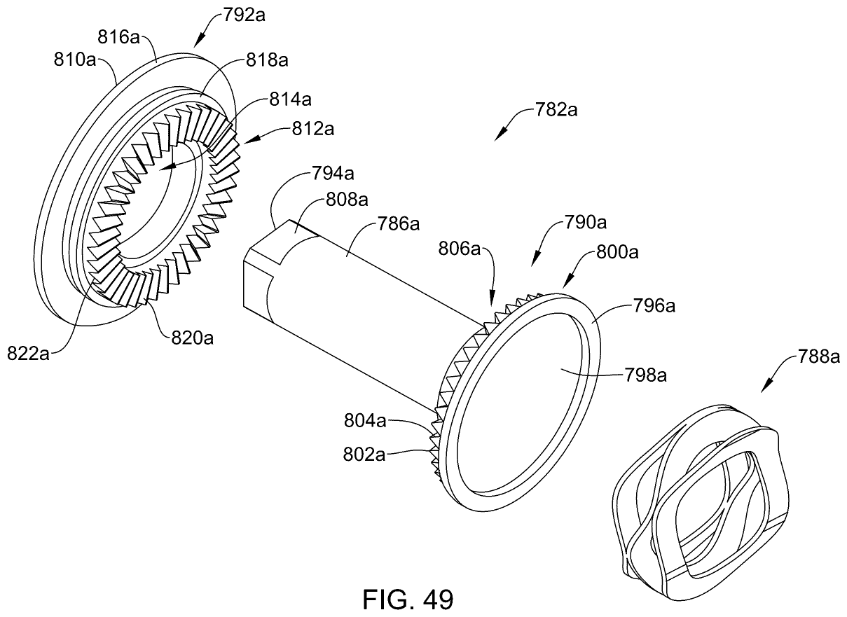

The gear assembly housing 763 may further include a first or left cylindrical member 774a and a second or right cylindrical member 774b coupled to and extending vertically from the central beam 772. The first cylindrical member 774a and the second cylindrical member 774b may each define a cavity 776a, 776b therein (see, for example, FIG. 47C). For example, a laterally inward end 778a, 778b of the first and second cylindrical members 774a, 774b may be substantially solid while a laterally outward end 780a, 780b of the first and second cylindrical members 774a, 774b may define an opening. The cavity 776a, 776b may extend from the laterally outward end 780a, 780b towards the laterally inward end 778a, 778b of each of the respective cylindrical members 774a, 774b. The cavities 776a, 776b may each be configured to house a gear assembly 782a, 782b (see, for example, FIG. 47C and FIG. 49) configured to selectively couple the needle mount member 716 relative to the gear assembly member 718. The left and right cylindrical members 774a, 774b may be laterally spaced from one another by a gap 784. The gap 784 may be laterally aligned with the aperture 760 of the needle mount member 716 and sized and shaped to receive a portion of the guide needle 172 therethrough, such as, but not limited to the elongate needle region 182. For example, the gap 784 may have a lateral width that is greater than the diameter of the elongated needle portion 182 of the guide needle 172 to allow the elongated needle portion 182 to pass therethrough. The gap 784 may extend from a top portion of the left and right cylindrical members 774a, 774b to the top surface of the central beam 772.

The gear assembly member 718 may further include a first or left mounting member 786a extending laterally outwards from a laterally outward end 780a of the left cylindrical member 774a and a second or right mounting member 786b extending laterally outwards from a laterally outward end 780b of the right cylindrical member 774b. The left and right mounting members 786a, 786b may be a part of the gear assemblies 782a, 782b. The left and right mounting members 786a, 786b may be configured to be received within the apertures 758a, 758b of the needle mount member 716, as will be described in more detail herein.

FIG. 49 is an exploded view of an illustrative gear assembly 732a. The right gear assembly 732b may include the same components in a mirror image arrangement to the left gear assembly 732a. The gear assembly 732a may include biasing mechanism 788a, a first gear 790a, and a second gear 792a. The biasing mechanism 788a may be a compression spring, a wave spring washer, a leaf spring, or the like. The first gear 790a may extend from a first or left end 794a to a second or right end 796a. The first gear 790a may include a disc member 800a at the second end 796a thereof. The disc member 800a may have a recess 798a formed in the right end surface thereof. The recess 798a may be sized and shaped to receive an end of the biasing mechanism 788a therein. The recess 798a may extend through less than an entire thickness of the disc member 800a. It is contemplated that a diameter of the recess 798a may be the same as or similar to a diameter of the biasing mechanism 788a to help prevent unintended movement of the biasing mechanism 788a relative to the first gear 790a. The disc member 800a may further include a plurality of ridges 802a and valleys 804a (e.g., gear teeth) formed in or extending from a left end surface thereof, where not all of the ridges 802a and valleys 804a have been identified in the Figures for brevity and ease of understanding. The ridges 802a and valleys 804a may form an annular ring. I

The mounting member 786a may extend laterally outward from the left end surface of the disc member 800a. The mounting member 786a may be positioned within the annular ring of the ridges 802a and valleys 804a. The mounting member 786a may include a first region 806a connected to the disc member 800a having a first cross-sectional shape, such as, but not limited to, circular and a second region 808a having a second cross-sectional shape, such as, but not limited to, square. The diameter of the first region 806a may be similar to a diameter of a lumen 814a of the second gear 792a to help prevent unintended movement of the mounting member 786a relative to the second gear 792a. The second region 808a may be adjacent to the left end 794a of the first gear 790a. The second region 808a may have a non-circular cross-sectional shape. For example, the cross-sectional shape of the second region 808a may be keyed, include planar regions, etc. such that rotational movement of the needle mount member 716 may be selectively translated to the first gear 790a. For example, the mounting member 786a of the first gear 790a may be configured to extend through the second gear 792a and into the through hole 758a of the left arm 750 of the needle mount member 716. The through hole 758a may have a cross-section that changes shape to mate with the mounting member 786a of the first gear 790a. For example, the through hole 758a may include a first region 753a having a generally circular cross-sectional shape and a second region 755a having a generally square cross-sectional shape. Similarly, the through hole 758b of the right arm 752 may include a first region 753b having a generally circular cross-sectional shape and a second region 755b having a generally square cross-sectional shape. It is contemplated that the second region 755a may have a cross-sectional shape that mates with the second region 808a of the mounting member 786a.

The second gear 792a may extend from a first or left end 810a to a second or right end 812a. The second gear 792a may include a lumen 814a extending from the left end 794a to the right end 812a. The lumen 814a may be configured to receive the mounting member 786a of the first gear 790a therethrough. The second gear 792a may include an annular flange 816a at the left end 710a thereof. The annular flange 816a may have an outer diameter that is greater than an inner diameter of the cavity 776a of the first cylindrical member 774a. In some cases, the annular flange 816a may have an outer diameter that is approximately the same as the outer diameter of the first cylindrical member 774a. The laterally inward surface of the annular flange 816a may be fixed or removably secured to the left lateral surface of the first cylindrical member 774a such that rotation of the second gear 792a relative to first cylindrical member 774a (and the gear assembly housing 763) is precluded. The second gear 792a may further include an annular portion 818a extending laterally inward from the annular flange 816a. The annular portion 818a may have an outer diameter that is approximately the same as the inner diameter of the cavity 776a adjacent the right end thereof to provide a friction fit. However, this is not required. The outer diameter of the annular portion 818a may be less than the inner diameter of the cavity 776a adjacent the right end thereof.

The annular portion 818a may further include a plurality of ridges 820a and valleys 822a (e.g., gear teeth) extending from a left end surface thereof, where not all of the ridges 820a and valleys 822a have been identified in the Figures for brevity and ease of understanding. The ridges 820a and valleys 822a may form an annular ring. In some embodiments, the outer diameter of the second gear 792a adjacent to the ridges 820a and valleys 822a may be less than an outer diameter at the annular portion 818a. However, this is not required. The ridges 820a and valleys 822a may be configured to mate with and selectively engage the ridges 802a and valleys 804a of the first gear 790a. When the 820a and valleys 822a of the second gear 792a are engaged with the ridges 802a and valleys 804a of the first gear 790a rotation of the needle mount member 716 may be precluded. For example, as the second gear 792a is rotationally fixed, the first gear 790a which is rotationally coupled to the needle mount member 716 cannot rotate when the gear teeth 802a, 804a, 820a, 822a are engaged thus preventing swivel (angular) movement of the needle mount member 716 and the guide needle 172. The same concept applies to the second gear assembly 782b.

When assembled within the cavity 776a of the first cylindrical member 774a, the biasing mechanism 788a may be positioned between the interior wall 775a of the cavity 776a and the right end 796a of the first gear 790a. When it is desired to pivot or angle the guide needle 172, the user may pinch or bias the left and right arms 750, 752 laterally inwards to move the needle mount member 716 and the gear assemblies 782a, 782b into an unlocked configuration. The laterally inward force of the left arm 750 may deflect the first gear 790a laterally inwards (e.g., toward the interior wall 775a of the cavity 776a, 776b). For example, the transition between the first region 753a and the second region 755a of the through hole 758a, 758b may contact the transition between the second region 808a and the first region 806a of the mounting member 786a to transfer the lateral movement of the arm 750 to the mounting member 786a. As the first gear 790a is moved laterally inwards, the biasing mechanism 788a compresses and the first gear 790a is moved away from the second gear 792a to disengage the ridges 802a and grooves 804a of the first gear 790a from the ridges 820a and grooves 822a of the second gear 792a. With the gear teeth 802a, 804a of the first gear 790a spaced from the gear teeth 820a, 822a of the second gear 792a, the needle mount member 716 may be rotated to adjust an angular position of the guide needle 172. The same movement may occur within the second gear assembly 782b to compress the biasing member 788b and move the first gear 790b laterally inwards away from the second gear 792b.

This may allow the needle mount member 716 to allow the elongate needle region 182 to be angled relative to the horizontal axis 702. For example, the needle mount member 716 may be rotated to allow the elongate needle region 182 of the guide needle 172 to extend at an angle non-parallel to the horizontal axis 702. It is contemplated that the extent to which the guide needle 172 may be offset (up or down) from the horizontal axis may be determined at least in part by a vertical position of the needle actuation assembly 720 and/or the central beam 772. When the guide needle 172 is in the desired position, the biasing force on the arms 750, 752 may be removed such that the biasing mechanisms 788a, 788b pushes the first gears 790a, 790b back into engagement with the second gears 792a, 792b to prevent further rotation of the needle mount member 716.

Referring additionally to FIG. 50, which is a partial cross-sectional view of the needle actuation assembly 720 movably coupled with the vertical support 712, the guide system 700 may further include a first or left actuation member 824a and a second or right actuation member 824b. The left and right actuation members 824a, 824b may have a same structure. The actuation members 824a, 824b may include a push button 826a, 862b, an elongate shaft 828a, 828b, and a generally “L” shaped member 830a, 830b extending between the elongate shaft 828a, 828b and the push button 826a, 826b. The push button 826a, 862b may include features configured to increase the gripability or friction thereof. For example, the push button 826a, 862b may include grooves, bumps, protrusions, slots, etc. along an outer surface thereof. The “L” shaped member 830a, 830b may include a first region extending proximally and a second region extending in a downward direction at a generally orthogonal angle to the first region. This may vertically and axially offset the push buttons 826a, 826b from the free ends of the left and right arms 750, 752 of the needle mount member 716 such that the push buttons 826a, 826b are easily accessible.

The elongate shaft 828a, 828b may extend laterally inwards from the first region of the generally “L” shaped member 830a, 830b. An axial thickness of the elongate shaft 828a, 828b may vary along the lateral width thereof. For example, the elongate shaft 828a, 828b may incrementally decreased over one or more sections in axial length until a laterally inward most enlarged region 832a, 832b. The enlarged region 832a, 832b may gradually increase in axial length towards the laterally inward end. In some cases, the gradual increase may be a linear slope. In other examples, the gradual increase may be a curve (e.g., like a hemisphere). The elongate shaft 828a, 828b may be configured to extend through the left and right channels 736a, 736b of the vertical support 712 and into the left and right apertures 771a, 771b of the grooves 764a, 764b of the gear assembly member 718. The legs 770a, 770b of the gear assembly member 718 are slidably disposed within the second region 742a, 742b of the left and right channels 736a, 736b, respectively.

A biasing member 834a, 834b may be positioned within the left and right apertures 771a, 771b between an interior wall thereof and a laterally inward end of the elongate shaft 828a, 828b. The biasing members 834a, 834b may be a compression spring, a wave spring washer, a leaf spring, or the like. The biasing members 834a, 834b may be configured to bias the elongate shaft 828a, 828b in a laterally outward direction. When the biasing members 834a, 834b are biasing the elongate shaft 828a, 828b in a laterally outward direction, the enlarged regions 832a, 832b are in contact with and pushing against the brake members 836a, 836b, 838a, 838b. A first pair of brake members 836a, 836b may be positioned on a proximal side and a distal side of the first elongate shaft 828a. Similarly, a second pair of brake members 838a, 838b may be positioned on a proximal side and a distal side of the second elongate shaft 828b. The laterally enlarged regions 832a, 832b push the proximal brake members 836a, 838a (slidably disposed within the axially extending channels 773a, 773b of the gear assembly member 718) proximally into the proximal face 732e, 734e of the left and right channels 736a, 736b and the distal brake members 836b, 838b (slidably disposed within the axially extending channels 773a, 773b of the gear assembly member 718) into the distal face 732f, 734f of the left and right channels 736a, 736b. The frictional and/or mechanical engagement between the proximal brake members 836a, 838a and the proximal face 732e, 734e and between the distal brake members 836b, 838b and the distal face 732f, 734f maintain the needle actuation assembly 720 at a desired vertical height. The proximal end surfaces of the proximal brake members 836a, 838a may mirror the proximal faces 732e, 734e at the second regions 742a, 742b of the channels 736a, 736b to increase frictional engagement between the two components. Similarly, the distal end surfaces of the distal brake members 836b, 838b may mirror the distal faces 732f, 734f at the second regions 742a, 742b of the channels 736a, 736b to increase frictional engagement between the two components. The biasing force of the biasing members 834a, 834b may be overcome by exerting a laterally inwards force on the push buttons 826a, 826b. This may compress the biasing member 834a, 834b to allow the elongate shafts 828a, 828b to move laterally inwards. This may position an intermediate region 840a, 840b of the elongate shaft 828a, 828b having an axial length less than the axial length of the enlarged region 832a, 832b. Once the intermediate region 840a, 840b is aligned with the distal end of the proximal brake members 836a, 838a and the proximal end of the distal brake members 836b, 838b, the proximal brake members 836a, 836b may shift distally and the distal brake members 836b, 838b to disengage from the proximal faces 732e, 734e and distal faces 732f, 734f, respectively. The biasing force may be maintained on the push buttons 826a, 826b as the needle actuation assembly 720 is moved up and/or down within the vertical support 712. When the needle actuation assembly 720 is at the desired vertical location, the biasing force on the push buttons 826a, 826b may be releases to reengage the brake members 836a, 836b, 838a, 838b with the proximal and distal faces 732e, 734e, 732f, 734f of the left and right channels 736a, 736b and maintain the needle actuation assembly 720 at the desired height.

In use, the guide system 700 may be secured to an ultrasound probe using a strap (although this is not required) before or after the ultrasound probe is positioned within the anatomy. For example, for a prostate biopsy, the ultrasound probe may be positioned within the rectum. Once the guide system 700 is secured to the ultrasound probe and the ultrasound probe is positioned, the vertical support 712 may be axially displaced along the horizontal slide member 710 and secured in the desired axial position. This may be performed with the locking mechanism 714 in the unlocked configuration and then moved to the locked configuration. Next, a vertical position of the needle actuation assembly 720 (and thus a vertical position of the guide needle 172) may be adjusted. The push buttons 826a, 826b may be biased laterally inwards to allow the needle actuation assembly 720 to be moved vertically within the channel of the vertical support 712. Once the needle actuation assembly 720 is at the desired height or vertical location, the push buttons 826a, 826b may be released. The arms 750, 752 of the needle mount member 716 may be biased laterally inwards and the needle mount member 716 rotated to position the guide needle 172 at a desired angle. Once the needle mount member 716 is in the desired position, the biasing force on the left and right arms 750, 752 may be released.

It is contemplated that multiple samples may be taken with the needle actuation assembly 720 at a same height. For example, the angle of the guide needle 172 may be adjusted to obtain multiple samples within a same plane. It is further contemplated that the order of the steps described herein may be rearranged. For example, the vertical and/or angular position of the guide needle 172 may be adjusted prior to moving the vertical support 712 along the horizontal slide member 710 towards or away from the patient's body.

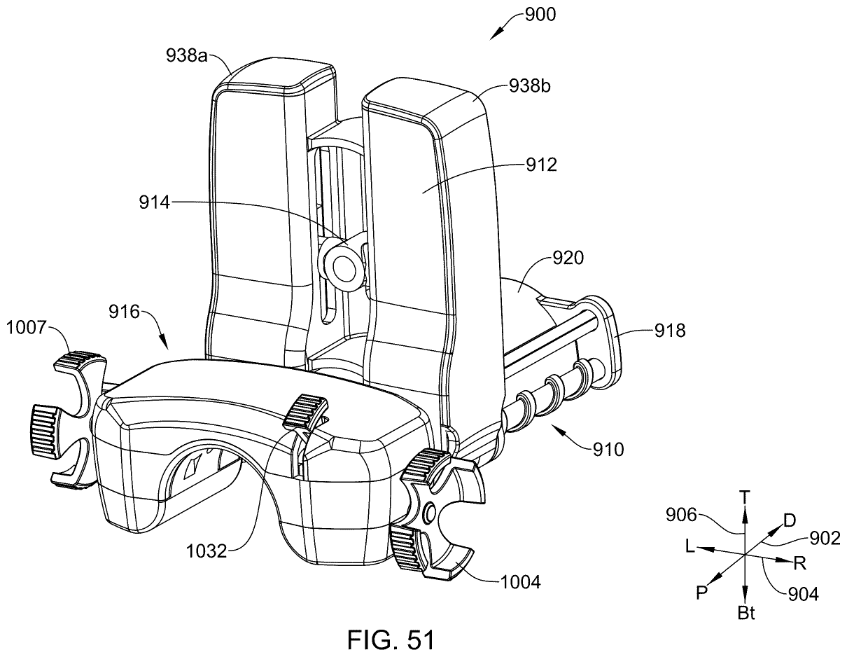

FIG. 51 is a perspective view another illustrative prostate biopsy guide system 900 in a first configuration. While the guide system 900 is described with respect to prostate biopsy, it is contemplated that the guide system 900 may be used in other procedures. In one illustrative example, the guide system 900 may be used to facilitate the injection of a spacing material, such as between the rectum and the prostate to prevent damage during radiation therapy. One illustrative system for injecting materials into the space between the rectum and the prostate is the SpaceOAR Vue™ System available from Boston Scientific Corporation, Marlborough, MA, USA. The guide system 900 may be configured to allow for horizontal, vertical, and angular movement of the biopsy needle (not explicitly shown) while also fixing the biopsy needle in a particular configuration. The guide system 900 may be configured to mount to an ultrasound probe (not explicitly shown) such that the guide system 900 is in a generally fixed position relative to the ultrasound probe. As used herein, movement in the horizontal direction or relational positioning in a direction generally parallel to axis 902, will be described as axial movement or an axial direction with relational positioning using proximal (P) (or front) and distal (D) (or back); movement or relational positioning in a direction generally parallel to axis 904 will be described as lateral movement or a lateral direction with relational positioning using left (L) and right (R); and movement or relational positioning in a direction generally parallel to axis 906 will be described as vertical movement or a vertical direction with relational positioning using top (T) and bottom (Bt). The use of the terms “proximal”, “distal”, “front,” “back”, “left”, “right”, “top”, “bottom”, “up”, and “down” are not intended to limit the guide system 900 to a particular orientation, but rather facilitate discussion of relative orientation.

Generally, the guide system 900 may be configured to control a horizontal position, a vertical position, and/or an angle of a guide needle. The guide system 900 may be configured to lock the guide needle (not explicitly shown, similar in form and function to guide needle 172 as shown and described with respect to FIG. 9) in a desired orientation independent of an ultrasound probe. It is contemplated that guide needles having alternative configurations may also be used with the guide system 900. Further, the vertical and/or angular position may be held as the guide needle is moved towards the patient's body and/or away from the patient's body. The guide system 900 may include a horizontal support member 910, a vertical housing 912, and a needle mount member 914, among other features. The needle mount member 914 may be pivotable relative to the vertical housing 912 to change an angle of the guide needle. Further, the needle mount member 914 may be configured to move up and down along the vertical housing 912 to control a vertical position of the needle mount member 914 (and thus the guide needle). The vertical housing 912 may be moved along the horizontal support member 910 to move the guide needle distally towards and/or proximally away from the patient.

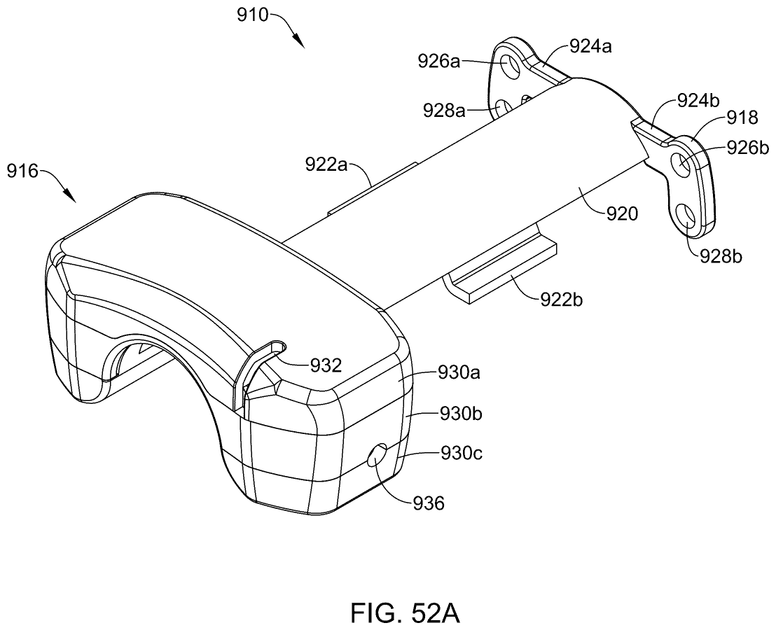

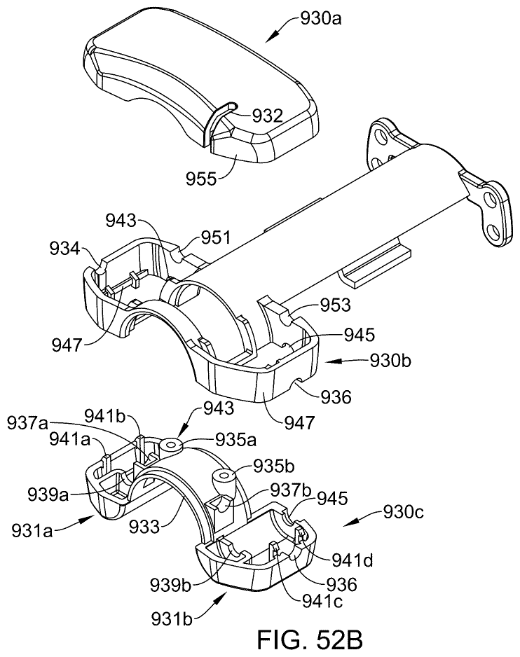

FIG. 52A is a perspective view of the illustrative horizontal support member 910. The horizontal support member 910 may include a proximal housing 916, a distal bracket 918, and an intermediate support region 920 extending between the proximal housing 916 and the distal bracket 918. The proximal housing 916 may define an interior cavity configured house a plurality of gears and actuation mechanisms configured to independently control vertical, horizontal, and pivot movement of the needle mount member 914. The intermediate support region 920 may have concave bottom surface configured to conform to an outer surface of the ultrasound probe. For example, the concave lower surface may rest on an outer surface of the ultrasound probe. A first or left support member 922a may extend laterally from a left side of the intermediate region 920 and a second or right support member 922b may extend laterally from a right side of the intermediate region 920. The support members 922a, 922b may help support the vertical housing 912. The distal bracket 918 may include a first or left arm 924a and a second or right arm 924b. The left arm 924a may extend laterally outward from a left lateral side of the intermediate support region 920 and the right arm 924b may extend laterally outward from a right lateral side of the intermediate support region 920. Each of the left and right arms 924a, 924b may include a laterally extending region which extends parallel to axis 904 and a vertically extending region which extends parallel to axis 906.

A first or upper recess 926a and a second or lower recess 928a may be formed in the vertically extending region of the left arm 924a. The upper recess 926a may be positioned adjacent to a top of the left arm 924a while the lower recess 928a may be positioned adjacent to a bottom of the left arm 924a. The upper and lower recesses 926a, 928a may extend through less than an entire thickness of the left arm 924a. However, this is not required. The upper and lower recesses 926a, 928a may extend through an entire thickness of the left arm 924a, if so desired.

A first or upper recess 926b and a second or lower recess 928b may be formed in the vertically extending region of the right arm 924b. The upper recess 926b may be positioned adjacent to a top of the right arm 924b while the lower recess 928b may be positioned adjacent to a bottom of the right arm 924b. The upper and lower recesses 926b, 928b may extend through less than an entire thickness of the right arm 924b. However, this is not required. The upper and lower recesses 926b, 928b may extend through an entire thickness of the right arm 924b, if so desired.

As will be described in more detail herein, the left upper recess 926a may be configured to receive a distal end of a vertical travel shaft 1021. The right upper recess 926b may be configured to receive a distal end of a swivel shaft 1046. The left lower recess 928a may be configured to receive a distal end of a support shaft 1094. The right lower recess 928b may be configured to receive a distal end of a horizontal travel lead screw 1022.

The proximal housing 916 may include a top cover 930a, an intermediate cover 930b, and a bottom cover 930c. The intermediate cover 930b may be formed as a single monolithic structure with the intermediate support region 920 and the distal bracket 918. However, this is not required. The intermediate cover 930b may be formed as a separate component from the intermediate support region 920 and/or the distal bracket 918 and subsequently coupled thereto. The top cover 930a may be releasably coupled to an upper portion of the intermediate cover 930b using, for example, snap fits, friction fits, or the like. Similarly, the bottom cover 930c may be releasably coupled to a lower portion of the intermediate cover 930b using, for example, snap fits, friction fits, or the like. The top and bottom covers 930a, 930c may be formed separately from the intermediate cover 930b and subsequently assembled therewith to allow gear and actuation mechanisms to be positioned within the proximal housing 916. The proximal housing 916 may include a plurality of slots or apertures extending through a wall thickness thereof and configured to allow actuation members to pass from an interior cavity of the proximal housing 916 to an exterior thereof. For example, the proximal housing 916 may include a slot 932 extending from a top side of the top cover 930a to a proximal side of the top cover 930a. The slot 932 may allow a pivot control knob 1032 to be displaced therein to control an angle of the needle mount member 914, as will be described in form detail herein. A first or left aperture 934 (see, for example, FIG. 52B) may be formed through a left side wall of the proximal housing 916. In some cases, the left aperture 934 may be formed in part by aligning a recess in the top cover 930a and a recess in the intermediate cover 930b. However, this is not required. An elongate shaft 1009 coupled to a vertical control knob 1007 may extend through the left aperture 934 to control a vertical position of the needle mount member 914. A second or right aperture 936 may be formed through a right-side wall of the proximal housing 916. In some cases, the right aperture 936 may be formed in part by aligning a recess in the bottom cover 930c and a recess in the intermediate cover 930b. However, this is not required. An elongate shaft 1006 coupled to a horizontal control knob 1004 may extend through the right aperture 936 to control a horizontal or axial (e.g., proximal to distal) position of the needle mount member 914.

FIG. 52B is an exploded perspective view of the horizontal support member 910. The bottom cover 930c may include a first or left housing member 931a and a second or right housing member 931b. The left and right housing members 931a, 931b may have a generally solid bottom and a generally open top to define a recess or cavity therein. The left and right housing members 931a, 931b may be interconnected by a curved bridge member 933. The curved bridge member 933 may be sized and shaped to rest on an outer surface of an ultrasound probe. The curved bridge member 933 may include apertures 935a, 935b configured to align with and/or couple with mating features (not explicitly shown) of the top cover 930a. The curved bridge member 933 may further include a first curved recess 937a configured to receive a portion of the elongate shaft 1009 of the third gear and actuation assembly 1000. The first curved recess 937a may be curved to allow the elongate shaft 1009 to rotate relative to the first curved recess 937a. The curved bridge member 933 may further include a second curved recess 937b configured to receive a portion of the elongate shaft 1034 of the second gear and actuation assembly 998. The second curved recess 937b may be curved to allow the elongate shaft 1034 to rotate relative to the second curved recess 937b.

The left housing member 931a may include a curved support 939a configured to receive a proximal end region of the support shaft 1094 thereon. The left housing member 931a may include a curved support 939b configured to receive a proximal end region of the horizontal travel lead screw 1022 of the first gear and actuation assembly 996. The curved support 939b may be curved to allow the horizontal travel lead screw 1022 to rotate relative to the curved support 939b. The bottom cover 930c may include one or more latch members 941a-d configured to releasably engage a mating ledge 947a or other feature of the intermediate cover 930b to couple the top portion of the bottom cover 930c with a bottom portion of the intermediate cover 930b. A first distal opening 943 may be formed through a distal wall of the left housing member 931a for allowing the support shaft 1094 to pass therethrough. A portion of the first opening 943 may also be defined by the intermediate cover 930b. A second distal opening 945 may be formed through a distal wall of the right housing member 931b for allowing the horizontal travel lead screw 1022 to pass therethrough. A portion of the second distal opening 945 may also be defined by the intermediate cover 930b.

The intermediate cover 930b may have a generally open top portion and a generally open bottom portion. Said differently, the intermediate cover 930b may include a side wall 947 configured to extend around a perimeter of the proximal housing 916. The intermediate cover 930b may include a central opening 949 configured to receive the curved bridge member 933 therein. A third distal opening 951 may be formed through a distal wall of the intermediate cover 930b for allowing the vertical travel shaft 1021 to pass therethrough. A portion of the third distal opening 951 may also be defined by the top cover 930a. A fourth distal opening 953 may be formed through a distal wall of the intermediate cover 930b for allowing the swivel shaft 1046 to pass therethrough. A portion of the fourth distal opening 953 may also be defined by the top cover 930a.

The top cover 930a may include a generally solid top portion and a generally open bottom portion. A side wall 955 configured to extend around a perimeter of the proximal housing 916 may extend downwards from the top portion. The bottom portion of the top cover 930a may be releasably coupled to the top portion of the intermediate cover 930b.

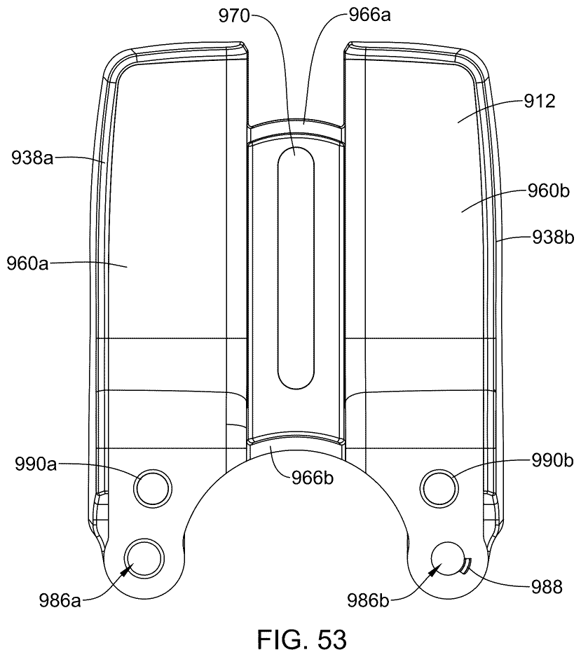

FIG. 53 is a proximal or front view of the vertical housing 912 include a first or left end cover 938a and a second or right end cover 938b. The end covers 938a, 938b may be releasably coupled to the vertical housing 912 to house actuation elements within the portions of the vertical housing 912. For example, the end covers 938a, 938b may be configured to cover side cavities 972a, 972b including actuation members therein. The actuation members may be assembled within the side cavities 972a, 972b and the end covers 938a, 938b may be subsequently coupled to the vertical housing 912 to house and protect the actuation members.

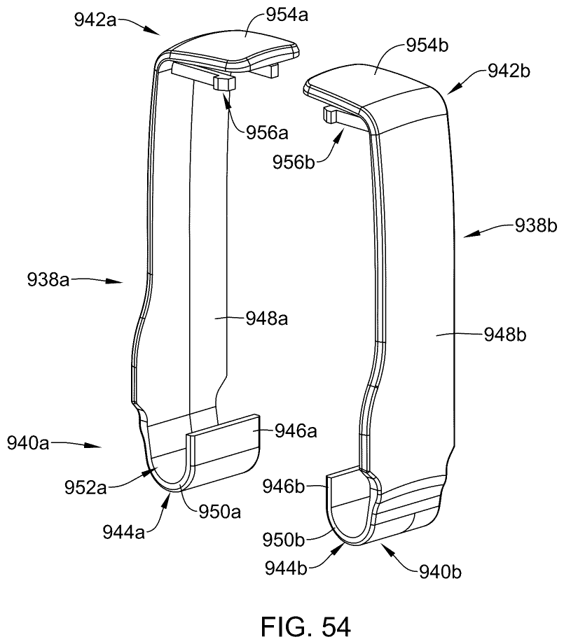

FIG. 54 is a perspective view of the left and right end covers 938a, 938b. Each of the end covers 938a, 938b may extend from a bottom end region 940a, 940b to a top end region 942a, 942b. The end covers 938a, 938b may have a generally plate-like structure with curves or bends adjacent to the bottom end region 940a, 940b and the top end region 942a, 942b. The bottom end region 940a, 940b may be curved to from a generally “U” shaped region 944a, 944b including a laterally inward leg 946a coupled to an intermediate region 948a, 948b of the end cover 938a, 938b via a curved member 950a, 950b. The laterally inward leg 946a, 946b may be laterally spaced from the intermediate region 948a, 948b to define a channel 952a, 952b extending from a proximal side to a distal side of the end cover 938a, 938b. The generally “U” shaped region 944a, 944b may be configured to be releasably coupled to a tubular member 984a, 984b of the vertical housing 912.

The top end region 942a, 942b may include a laterally extending arm 954a, 954b. The arm 954a, 954b may be configured to extend along an upper region 911a, 911b of the vertical housing 912. In some cases, the arm 954a, 954b may extend generally orthogonal to the intermediate region 948a, 948b. However, this is not required. The end cover 938a, 938b may further include a clamp member 956a, 956b. The clamp members 956a, 956b may each include a pair of arms configured to releasably grip a first and/or second post 982a, 982b of the vertical housing 912.

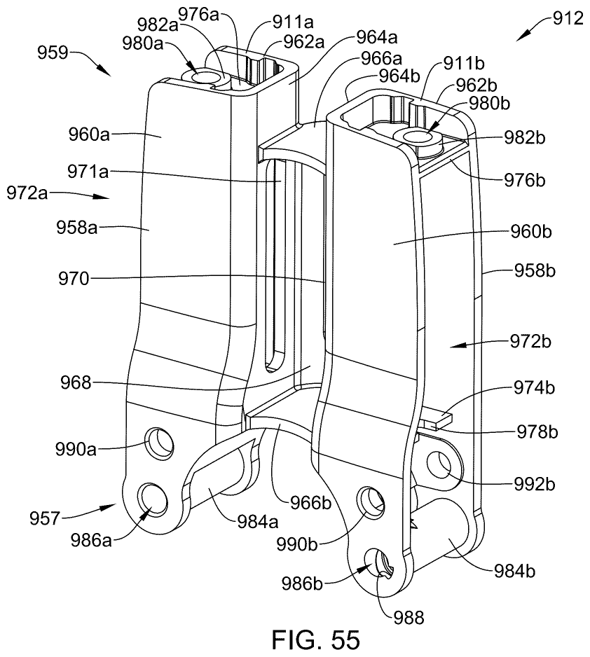

FIG. 55 is a perspective view of the illustrative vertical housing 912. The vertical housing 912 may extend from a bottom end region 957 to a top end region 959. The vertical housing 912 may include a first or left support member 958a and a second or right support member 958b. The left and right support members 958a, 958b may be mirror images of one another. Each of the left and right support members 958a, 958b may include a proximal or front panel 960a, 960b, a distal or back panel 962a, 962b extending generally parallel to the proximal panel 960a, 960b, and an interconnecting panel 964a, 964b extending between and generally orthogonal to the proximal panel 960a, 960b and the distal panel 962a, 962b. The interconnecting panel 964a of the left support member 958a may extend between the laterally inward or right ends of the proximal and distal panels 960a, 962a. The interconnecting panel 964b of the right support member 958b may extend between the laterally inward or left ends of the proximal and distal panels 960b, 962b.

An upper support member 966a may extend between the interconnecting panel 964a of the left support member 958a and the interconnecting panel 964b of the right support member 958b adjacent the top end region 959 of the vertical housing 912. In some cases, the upper support member 966a may be curved. However, this is not required. A lower support member 966b may extend between the interconnecting panel 964a of the left support member 958a and the interconnecting panel 964b of the right support member 958b vertically spaced in an upward direction from the bottom top end region 957 of the vertical housing 912. The lower support member 966b may be curved. The curve of the lower support member 966b may be similar to the curve of the intermediate support region 920 of the horizontal support member 910 to allow the vertical housing 912 to move axially proximally and distally along the horizontal support member 910.

In some cases, a back panel 968 may extend between the distal ends of the interconnecting panels 964a, 964b and the distal ends of the upper and lower support members 966a, 966b. A vertically extending slot 970 may extend through a thickness of the back panel 968. The slot 970 may have a lateral width greater than a diameter of the elongate needle portion of the guide needle to allow the elongate needle portion to pass therethrough. A vertical height of the slot 970 may be selected to allow a desired vertical and/or angular adjustment of the guide needle via the needle mount member 914. Vertically extending slots 971a, 971b may extend through a thickness of the left and right interconnecting panels 964a, 964b, respectively. The vertically extending slots 971a, 971b may be positioned vertically between the upper and lower support members 966a, 966b. A vertical height of the slots 971a, 971b may be selected to allow a desired vertical adjustment of the needle mount member 914.

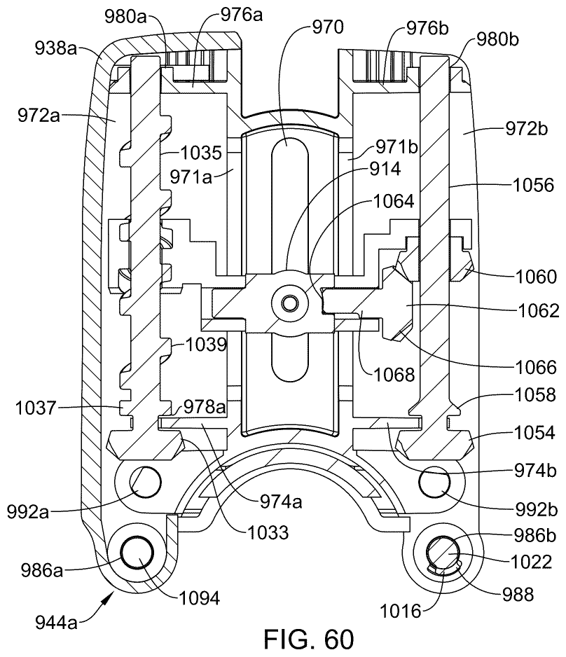

Each of the left and right support members 958a, 958b may define an opening or cavity therein 972a, 972b. The cavity 972a of the left support member 958a may be laterally accessible from a left side or lateral outer side thereof. The cavity 972b of the right support member 958b may be laterally accessible from a right side or lateral outer side thereof. Each of the cavities 972a, 972b may extend from a lower dividing wall 974a, 974b (see, for example, FIG. 60) to an upper dividing wall 976a, 976b. The lower dividing wall 974a, 974b may include a notch or cut-out region 978a, 978b (see, for example, FIG. 60) configured to allow a vertical travel lead screw 1035 and/or elongate shaft 1056 to pass therethrough. The upper dividing wall 976a, 976b may include an aperture 980a, 980b extending through a thickness thereof and configured to allow a vertical travel lead screw 1035 and/or elongate shaft 1056 to pass therethrough. The aperture 980a, 980b may be surrounded or ringed by a vertically extending annular post 982a, 982b. The clamp member 956a, 956b of the end covers 938a, 938b may be configured to be positioned around an outer surface of the annular posts 982a, 982b to releasably secure the end covers 938a, 938b to the upper end region 959 of the vertical housing 912.

The vertical housing 912 may further include a left tubular member 984a defining a lumen 986a extending from a proximal end to a distal end of the tubular member 984a. The lumen 986a may be configured to receive the support shaft 1094 therethrough. The vertical housing 912 may further include a right tubular member 984b defining a lumen 986b extending from a proximal end to a distal end of the tubular member 984b. The lumen 986b may be configured to receive the horizontal travel lead screw 1022 therethrough. Further, a helically extending groove 988 may be formed in the luminal wall. The helically extending groove 988 may be configured to receive and mate with a corresponding helically extending thread 1016 formed on an outer surface of the horizontal travel lead screw 1022 to facilitate axial (horizontal) movement of the vertical housing 912 relative to the horizontal support member 910.

A first proximal aperture 990a may be formed in the proximal panel 960a of the left support member 958a. The first proximal aperture 990a may be vertically positioned between the lower dividing wall 974a and the left tubular member 984a. A first distal aperture 992a (see, for example, FIG. 60) may be formed in the distal panel 962a of the left support member 958a. The first distal aperture 992a may be vertically and laterally aligned with the first proximal aperture 990a. The first proximal aperture 990a and the first distal aperture 992a may be configured to receive the vertical travel shaft 1021 therethrough.

A second proximal aperture 990b may be formed in the proximal panel 960b of the right support member 958b. The second proximal aperture 990b may be vertically positioned between the lower dividing wall 974b and the right tubular member 984b. A second distal aperture 992b may be formed in the distal panel 962b of the right support member 958b. The second distal aperture 992b may be vertically and laterally aligned with the second proximal aperture 990b. The second proximal aperture 990b and the second distal aperture 992b may be configured to receive the swivel shaft 1046 therethrough.

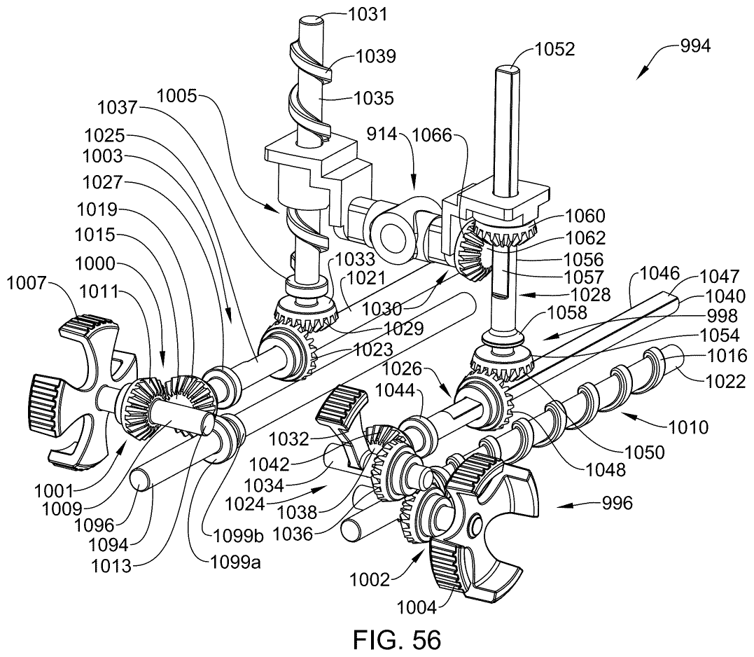

FIG. 56 is a perspective view of an illustrative gear and actuation assembly 994 that is disposed within the proximal housing 916 and the vertical housing 912 to control axial, vertical, and angular positioning of the needle mount member 914. The gear and actuation assembly 994 may include a first gear and actuation assembly 996 for controlling an axial position (proximal to distal) of the vertical housing 912 (and thus the needle mount member 914), a second gear and actuation assembly 998 for controlling an angular position of the needle mount member 914, and a third gear and actuation assembly 1000 for controlling a vertical position (top to bottom) of the needle mount member 914. Each of the first gear and actuation assembly 996, second gear and actuation assembly 998, third gear and actuation assembly 1000 may be controlled independently from control knobs 1004, 1032, 1007 adjacent to the proximal housing 916 of the guide system 900.

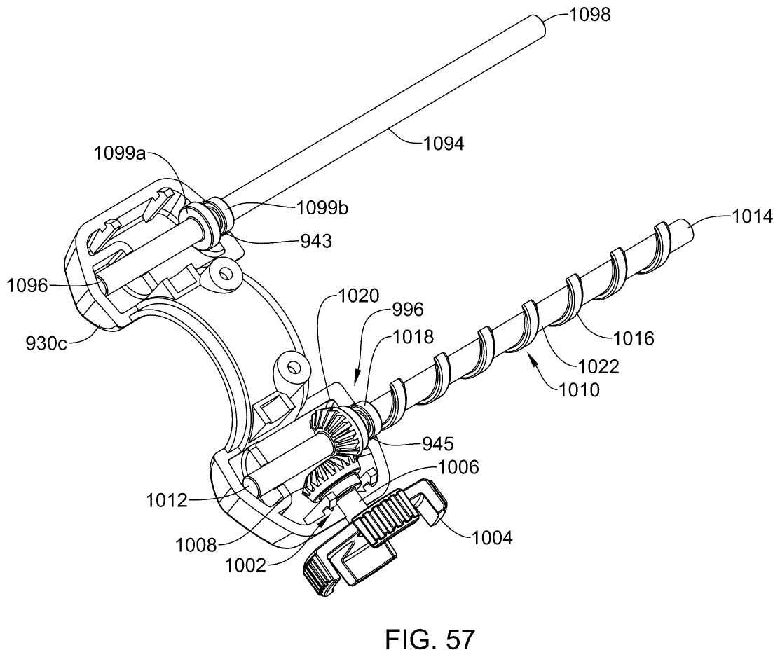

FIG. 57 is a perspective view of the first gear and actuation assembly 996 and the support shaft 1094 disposed with the bottom cover 930c of the proximal housing 916. The first gear and actuation assembly 996 may include a first gear assembly 1002 including a horizontal control knob (or another actuation member) 1004, an elongate shaft 1006, and a first bevel gear 1008. The horizontal control knob 1004 may be positioned exterior to the proximal housing 916. The elongate shaft 1006 may extend from the horizontal control knob 1004, through the opening 936 in the proximal housing 916 to the first bevel gear 1008 which is disposed within the proximal housing 916. The elongate shaft 1006 may extend generally parallel to axis 904 and may rotatably couple the horizontal control knob 1004 with the first bevel gear 1008.

The first gear and actuation assembly 996 may include a second gear assembly 1010 moveably coupled to the first gear assembly 1002. The second gear assembly 1010 may extend from a proximal end 1012 to a distal end 1014. The proximal end 1012 may be positioned within an interior or cavity of the proximal housing 916 while the distal end 1014 may be received within the lower recess 928b of the distal bracket 918. The second gear assembly 1010 may extend through the second distal opening 945 of the proximal housing 916. In some cases, the second distal opening 945 may include a transition in diameter. For example, the diameter of the second distal opening 945 may be greater adjacent to the outer surface thereof than a diameter adjacent to an inner surface of the proximal housing 916. The larger diameter region of the second distal opening 945 may be configured to receive a stop member 1018 of the second gear assembly 1010. The stop member 1018 may have an outer diameter greater than the smaller diameter of the second distal opening 945 to limit proximal movement of the second gear assembly 1010. A raised helical thread 1016 may protrude radially outward from an outer surface of the second gear assembly 1010 between the stop member 1018 and the distal end 1014 thereof. The raised helical thread 1016 may form a horizontal travel lead screw 1022 extending generally parallel to axis 902. As described above, the horizontal travel lead screw 1022 may be configured to mate with the helically extending groove 988 of the vertical housing 912. As the horizontal travel lead screw 1022 is rotated, the vertical housing 912 is axially translated along a length of the horizontal travel lead screw 1022. The direction of travel of the vertical housing 912 may be controlled by a rotational direction of the horizontal control knob 1004.

The second gear assembly 1010 may further include a second bevel gear 1020 positioned between the stop member 1018 and the proximal end 1012 of the second gear assembly 1010. The second bevel gear 1020 may be disposed within the interior of the proximal housing 916 and configured to engage the first bevel gear 1008. Each of the first bevel gear 1008 and the second bevel gear 1020 may include conical gear teeth. The first bevel gear 1008 and the second bevel gear 1020 may be positioned at generally orthogonal angles to one another and arranged such that the conical gear teeth of the first bevel gear 1008 engage with the conical gear teeth of the second bevel gear 1020. As the horizontal control knob 1004 is rotated, the first bevel gear 1008 also rotates (e.g., rotation is around an axis parallel to axis 904). This rotation is translated to the second bevel gear 1020 which in turn causes the lead screw 1022 to rotate around an axis parallel to axis 902. The rotation of the horizontal travel lead screw 1022 is generally orthogonal to the rotation of the elongate shaft 1006. As the lead screw 1022 is rotated, the vertical housing 912 is axially translated along a length of the lead screw 1022. The direction of travel of the vertical housing 912 may be controlled by a rotational direction of the horizontal control knob 1004. For example, rotation in a first direction may cause distal movement of the vertical housing 912 while rotation in a second direction, opposite the first direction, may cause proximal movement of the vertical housing 912.

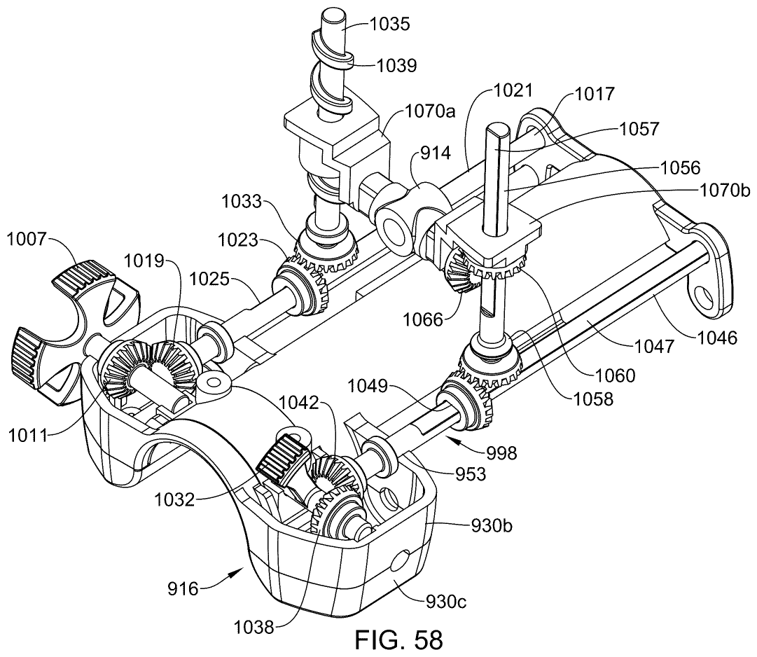

FIG. 58 is a perspective view of the second gear and actuation assembly 998 and the third gear and actuation assembly 1000 disposed within the intermediate cover 930b and bottom cover 930c of the proximal housing 916. The first gear and actuation assembly 996 is not shown in FIG. 58 to more particularly illustrate the second gear and actuation assembly 998. Referring to FIG. 56 and FIG. 58, the second gear and actuation assembly 998 may include a first gear assembly 1024, a second gear assembly 1026, a third gear assembly 1028, and a fourth gear assembly 1030. Generally, rotational movement may be translated from the first gear assembly 1024 to the second gear assembly 1026, then to the third gear assembly 1028, and ultimately to the fourth gear assembly 1030. The fourth gear assembly 1030 may be coupled to the needle mount member 914 such that as the fourth gear assembly 1030 rotates the needle mount member 914 is also rotated or pivoted to control an angle or swivel of the guide needle. This may allow the guide needle to be oriented at a parallel or non-parallel angle relative to the axis 902.

The first gear assembly 1024 may include a pivot control knob 1032 (or another actuation member), an elongate shaft 1034 extending generally parallel to axis 904, and a first bevel gear 1036. The pivot control knob 1032 may extend radially from an outer surface of the elongate shaft 1034 between a left and a right end thereof. The pivot control knob 1032 may be configured to pass through the slot 932 of the proximal housing 916 such that the pivot control knob 1032 is disposed exterior to the proximal housing 916. A user may actuate the pivot control knob 1032 along the slot 932. The length of the slot 932 may limit the movement of the pivot control knob 1032 to ultimately control how far or to what degree the guide needle is deflected up or down from an orientation parallel to the axis 902. The first bevel gear 1036 may extend around and be secured to the elongate shaft 1034 such that the first bevel gear 1036 rotates with the elongate shaft 1034 and may be positioned laterally between the pivot control knob 1032 and a right end of the elongate shaft 1034. The pivot control knob 1032 may include a plurality of conical teeth.

The second gear assembly 1026 may extend from a proximal end 1038 to a distal end 1040. A second bevel gear 1042 may be positioned at the proximal end 1038. The second bevel gear 1042 may include a plurality of conical teeth. The second bevel gear 1042 may be positioned generally orthogonal to the first bevel gear 1036 such that the plurality of conical teeth of the second bevel gear 1042 engage the plurality of conical teeth of the first bevel gear 1036 to allow rotational movement of the first bevel gear 1036 to be transferred to the second bevel gear 1042.

An elongate shaft or swivel shaft 1046 may extend parallel to axis 902 from the second bevel gear 1042 to the distal end 1040. The second bevel gear 1042 may be secured to the swivel shaft 1046 such that rotation of the second bevel gear 1042 causes rotation of the swivel shaft 1046. A third bevel gear 1048 may extend around the swivel shaft 1046 at a location between the second bevel gear 1042 and the distal end 1040. The third bevel gear 1048 may include a plurality of conical teeth. The third bevel gear 1048 may be positioned colinearly with the second bevel gear 1042. In some cases, the plurality of conical teeth of the third bevel gear 1048 may face in an opposite direction as the conical teeth of the second bevel gear 1042. However, this is not required. The third bevel gear 1048 may be axially displaceable along the swivel shaft 1046. The swivel shaft 1046 may include a flattened or keyed region 1047 configured to mate with a flattened region 1049 of a lumen of the third bevel gear 1048. This mating of the keyed regions 1047, 1049 may allow rotational force of the swivel shaft 1046 to be transferred to the third bevel gear 1048 without fixedly securing the third bevel gear 1048 to the swivel shaft 1046 such that axial movement of the third bevel gear 1048 remains possible.

The swivel shaft 1046 may extend through the fourth distal opening 953 of the proximal housing 916. A stop member 1044 may be positioned within an interior of the proximal housing 916. The stop member 1044 may have an outer diameter that is greater than a diameter of the fourth distal opening 953. The mechanical engagement between the stop member 1044 and the interior wall of the proximal housing 916 may limit distal movement of the second gear assembly 1026. The third bevel gear 1048 may positioned between the stop member 1044 and the distal end 1040 of the second gear assembly 1026.

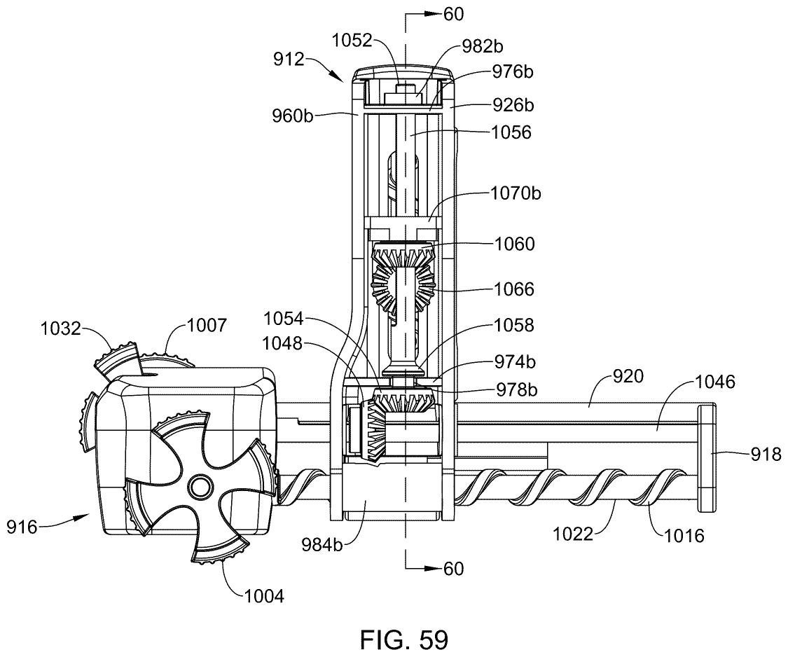

The swivel shaft 1046 may extend through the second proximal aperture 990b and the second distal aperture 992b of the vertical housing 912 with the third bevel gear 1048 positioned between the proximal panel 960b and the distal panel 962b of the right support member 958b. This may be more particularly illustrated in FIG. 59 which is a right-side view of the guide system 900 with the right end cover 938b removed to show the cavity 972b of the vertical housing 912. The third bevel gear 1048 may be axially slidable along the swivel shaft 1046 such that as the vertical housing 912 is proximally or distally moved relative to the horizontal support member 910, the third bevel gear 1048 moves with the vertical housing 912, as will be described in more detail herein.

The third gear assembly 1028 may extend from a bottom end 1050 to a top end 1052. A fourth bevel gear 1054 may be positioned at the bottom end 1050 of the third gear assembly 1028. The fourth bevel gear 1054 may include a plurality of conical teeth. The fourth bevel gear 1054 may be positioned generally orthogonal to the third bevel gear 1048 such that the plurality of conical teeth of the fourth bevel gear 1054 engage the plurality of conical teeth of the third bevel gear 1048 to allow rotational movement of the third bevel gear 1048 to be transferred to the fourth bevel gear 1054. An elongate shaft 1056 may be coupled to and extend vertically from the fourth bevel gear 1054 generally parallel to axis 906. The elongate shaft 1056 may be configured to pass through the cut-out region 978b and the aperture 980b. This may couple the third gear assembly 1028 to the vertical housing 912 such that as the vertical housing 912 is axially displaced the third gear assembly 1028 is also axially displaced. An inner surface (distal surface of proximal panel 960b) may contact a proximal end of the third bevel gear 1048 such that distal movement of the vertical housing 912 is also transferred to the third bevel gear 1048. Said differently, the third bevel gear 1048 may move distally with the vertical housing 912 (and the third gear assembly 1028) to maintain mechanical engagement between the third bevel gear 1048 and the fourth bevel gear 1054. Proximal movement of the vertical housing 912 may be transferred to the third bevel gear 1048 via the mechanical engagement between the third bevel gear 1048 and the fourth bevel gear 1054. For example, the fourth bevel gear 1054 may be located distal to the third bevel gear 1048 such that proximal movement of the fourth bevel gear 1054 is transferred to the third bevel gear 1048. However, this is not required.

A stop member 1058 may extend radially from and around the elongate shaft 1056 and may be vertically spaced from the fourth bevel gear 1054. The stop member 1058 may be configured to be positioned on top of or above the lower dividing wall 974b while the fourth bevel gear 1054 may be positioned below the lower dividing wall 974b of the vertical housing 912. The stop member 1058 may have an axial length that is greater than an axial length of the cut-out region 978b to prevent downwards movement of the third gear assembly 1028. The third gear assembly 1028 may further include a fifth bevel gear 1060 extending around the elongate shaft 1056 and positioned vertically between the stop member 1058 and the top end 1052. The fifth bevel gear 1060 may be configured to move up and down along the elongate shaft 1056 as the third gear and actuation assembly 1000 is actuated to control a vertical location of the needle mount member 914. The elongate shaft 1056 may include a flattened or keyed region 1057 configured to mate with a flattened region (not explicitly shown) of a lumen of the fifth bevel gear 1060. This mating of the keyed regions 1057 may allow rotational force of the elongate shaft 1056 to be transferred to the fifth bevel gear 1060 without fixedly securing the fifth bevel gear 1060 to the elongate shaft 1056 such that vertical movement of the fifth bevel gear 1060 remains possible.

The fourth gear assembly 1030 may extend from a right lateral end 1062 to a left lateral end 1064 (see, for example, FIG. 60). The fourth gear assembly 1030 may include a sixth bevel gear 1066 positioned at the right lateral end 1062. The sixth bevel gear 1066 may include a plurality of conical teeth. The sixth bevel gear 1066 may be positioned generally orthogonal to the fifth bevel gear 1060 such that the plurality of conical teeth of the sixth bevel gear 1066 engage the plurality of conical teeth of the fifth bevel gear 1060 to allow rotational movement of the fifth bevel gear 1060 to be transferred to the sixth bevel gear 1066. Referring additionally to FIG. 60 which is a cross-sectional view of the guide system 900, taken at line 60-60 of FIG. 59, the fourth gear assembly 1030 may include an elongate shaft 1068 extending laterally from the sixth bevel gear 1066. The elongate shaft 1068 may extend generally parallel to axis 904. The elongate shaft 1068 may extend into an aperture 917 of the needle mount member 914. The elongate shaft 1068 and the aperture 917 may have a keyed arrangement such that rotation of the elongate shaft 1068 is transferred to the needle mount member 914.

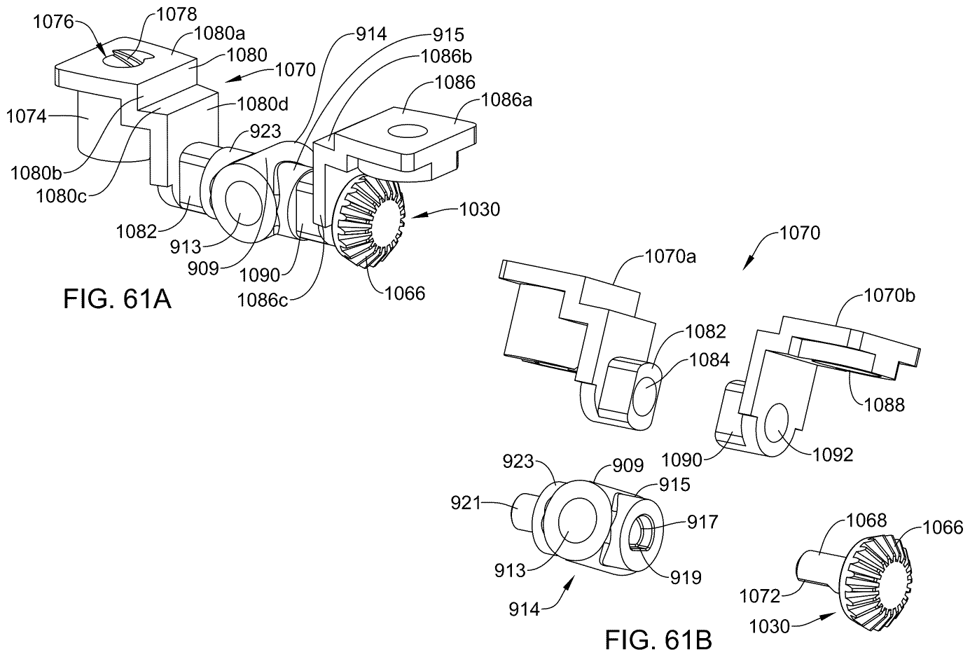

FIG. 61A is a perspective view of the fourth gear assembly 1030 assembled with the needle mount member 914 and a vertical slide assembly 1070. FIG. 61B is an exploded perspective view of the fourth gear assembly 1030, the needle mount member 914, and the vertical slide assembly 1070. The needle mount member 914 may include a generally tubular central body portion 909 defining an axially extending lumen 913 extending from a proximal end to a distal end thereof. The lumen 913 may be configured to receive at least a portion of a guide needle therethrough. The needle mount member 914 may further include a left body portion 915 defining an aperture 917 extending laterally inward from the right lateral side thereof. The aperture 917 may terminate before joining with the central lumen 913. However, this is not required. The aperture 917 may include a flattened region or keyed region 919. The keyed region 919 may be configured to mate with a flattened region or keyed region 1072 of the elongate shaft 1068 of the fourth gear assembly 1030 such that rotation of the elongate shaft 1068 is transferred to the needle mount member 914. The needle mount member 914 may further include a mounting member 921 extending laterally from a right body portion 923. The mounting member 921 may be a generally cylindrical member configured to be rotationally received within an aperture 1084 of the left body portion 1070a of the vertical slide assembly 1070. The mounting member 921 may rotate within the aperture 1084 to allow the needle mount member 914 to rotate while also allowing vertical movement of the vertical slide assembly 1070 to be transferred to the needle mount member 914.

The vertical slide assembly 1070 may include a first or left body portion 1070a and a second or right body portion 1070b. The left body portion 1070a may include a generally tubular member 1074 positioned at a laterally outward portion thereof. The tubular member 1074 may define a lumen 1076 extending from a bottom end to a top end thereof and generally parallel to axis 906. A helical extending groove 1078 may be formed in the luminal wall. The helical extending groove 1078 may be configured to receive and mate with a corresponding helically extending thread 1039 formed on an outer surface of a vertical travel lead screw 1035 to facilitate vertical movement of the needle mount member 914 relative to the vertical housing 912. For example, rotational movement of the vertical travel lead screw 1035 may cause corresponding vertical movement of the left body portion 1070a (and thus the needle mount member 914).

The left body portion 1070a may further include a stair-step bracket 1080 configured to couple the tubular member 1074 to a right body member 1082. The stair-step bracket 1080 may include one or more laterally extending plates 1080a, 1080c and one or more vertically extending plates 1080b, 1080d configured to couple the right body member 1082 to the tubular member 1074 in an orientation where the right body member 1082 is laterally and vertically offset from the tubular member 1074. An aperture 1084 may extend from a laterally outer edge of the right body member 1082. The aperture 1084 may extend though less that an entire lateral width of the right body member 1082. The aperture 1084 may have a generally circular cross-section configured to mate with the circular cross-section of the mounting member 921 and to allow for rotation of the mounting member 921 within the aperture 1084.

The right body portion 1070b may include a stair-step bracket 1086 including one or more laterally extending plates 1086a, 1086b and one or more vertically extending plates 1086c. A lower portion of an upper laterally extending plate 1086a may include a recess 1088 formed therein. The recess 1088 may be sized and shaped to receive a portion of the fifth bevel gear 1060. In some cases, the fifth bevel gear 1060 may be fixedly coupled to the upper laterally extending plate 1086a within the recess 1088. However, this is not required. A left body member 1090 may extend laterally from the vertically extending plate 1086c. A lumen 1092 may extend through the vertically extending plate 1086c and through the left body member 1090. The elongate shaft 1068 of the fourth gear assembly 1030 may extend through the lumen 1092 and into the aperture 917 of the needle mount member 914.

The right body member 1082 of the left body portion 1070a and the left body member 1090 of the right body portion 1070b may be configured to be slidably disposed within the vertically extending slots 971a, 971b of the vertical housing 912. For example, the right body member 1082 of the left body portion 1070a and the left body member 1090 of the right body portion 1070b may each have an axial length that is less than an axial length of the vertically extending slots 971a, 971b of the vertical housing 912.

Returning to FIGS. 56 and 58, the third gear and actuation assembly 1000 may include a first gear assembly 1001, a second gear assembly 1003, and a third gear assembly 1005. Generally, rotational movement may be translated from the first gear assembly 1001 to the second gear assembly 1003, and ultimately to the third gear assembly 1005. The third gear assembly 1005 may be coupled to the vertical slide assembly 1070 such that as the third gear assembly 1005 rotates the vertical slide assembly 1070 is vertically displaced to control a vertical position of the needle mount member 914. The first gear assembly 1001 may include a vertical control knob 1007 (or another actuation member), an elongate shaft 1009, and a first bevel gear 1011. The vertical control knob 1007 may be positioned exterior to the proximal housing 916. The elongate shaft 1009 may extend from the vertical control knob 1007, through the opening 934 in the proximal housing 916 to a right lateral end 1013. The first bevel gear 1011 may extend around and be coupled to the elongate shaft 1009 at a location between the vertical control knob 1007 and the end 1013. The elongate shaft 1009 may extend generally parallel to axis 904 and may rotatably couple the vertical control knob 1007 with the first bevel gear 1011. The first bevel gear 1011 may be disposed within the interior of the proximal housing 916.

The second gear assembly 1003 may extend from a proximal end 1015 to a distal end 1017. A second bevel gear 1019 may be positioned at the proximal end 1015. The second bevel gear 1019 may include a plurality of conical teeth. The second bevel gear 1019 may be positioned generally orthogonal to the first bevel gear 1011 such that the plurality of conical teeth of the second bevel gear 1019 engage the plurality of conical teeth of the first bevel gear 1011 to allow rotational movement of the first bevel gear 1011 to be transferred to the second bevel gear 1019.

A vertical travel shaft 1021 may extend parallel to axis 902 from the second bevel gear 1019 to the distal end 1017. The second bevel gear 1019 may be secured to the vertical travel shaft 1021 such that rotation of the second bevel gear 1019 causes rotation of the vertical travel shaft 1021. A third bevel gear 1023 may extend around the vertical travel shaft 1021 at a location between the second bevel gear 1019 and the distal end 1017. The third bevel gear 1023 may include a plurality of conical teeth. The third bevel gear 1023 may be positioned colinearly with the second bevel gear 1019. In some cases, the plurality of conical teeth of the third bevel gear 1023 may face in an opposite direction as the conical teeth of the second bevel gear 1019. However, this is not required. The third bevel gear 1023 may be axially displaceable along the vertical travel shaft 1021. The vertical travel shaft 1021 may include a flattened or keyed region 1025 configured to mate with a flattened region (not explicitly shown) of a lumen of the third bevel gear 1023. This mating of the keyed regions 1025 may allow rotational force of the vertical travel shaft 1021 to be transferred to the third bevel gear 1023 without fixedly securing the third bevel gear 1023 to the vertical travel shaft 1021 such that axial movement of the third bevel gear 1023 remains possible.

The vertical travel shaft 1021 may extend through the third distal opening 951 of the proximal housing 916. A stop member 1027 may be positioned within an interior of the proximal housing 916. The stop member 1027 may have an outer diameter that is greater than a diameter of the third distal opening 951. The mechanical engagement between the stop member 1027 and the interior wall of the proximal housing 916 may limit distal movement of the second gear assembly 1003. The third bevel gear 1023 may positioned between the stop member 1027 and the distal end 1017 of the second gear assembly 1003.

The vertical travel shaft 1021 may extend through the first proximal aperture 990a and the first distal aperture 992a of the vertical housing 912 with the third bevel gear 1023 positioned between the proximal panel 960a and the distal panel 962a of the left support member 958a in a similar manner to how the third bevel gear 1048 of the second gear and actuation assembly 998 is arranged. The third bevel gear 1023 may be axially slidable along the vertical travel shaft 1021 such that as the vertical housing 912 is proximally or distally moved relative to the horizontal support member 910, the third bevel gear 1023 moves with the vertical housing 912.

The third gear assembly 1005 may extend from a bottom end 1029 to a top end 1031. A fourth bevel gear 1033 may be positioned at the bottom end 1029 of the third gear assembly 1005. The fourth bevel gear 1033 may include a plurality of conical teeth. The fourth bevel gear 1033 may be positioned generally orthogonal to the third bevel gear 1023 such that the plurality of conical teeth of the fourth bevel gear 1033 engage the plurality of conical teeth of the third bevel gear 1023 to allow rotational movement of the third bevel gear 1023 to be transferred to the fourth bevel gear 1033. An elongate shaft or lead screw 1035 may extend generally parallel to axis 906 and may be coupled to and extend vertically from the fourth bevel gear 1033. The vertical travel lead screw 1035 may be configured to pass through the cut-out region 978a and the aperture 980a. This may couple the third gear assembly 1005 to the vertical housing 912 such that as the vertical housing 912 is axially displaced the third gear assembly 1005 is also axially displaced. An inner surface (e.g., distal surface of proximal panel 960a) may contact a proximal end of the third bevel gear 1023 such that distal movement of the vertical housing 912 is also transferred to the third bevel gear 1023. Said differently, the third bevel gear 1023 may move distally with the vertical housing 912 (and the third gear assembly 1005) to maintain mechanical engagement between the third bevel gear 1023 and the fourth bevel gear 1033. Proximal movement of the vertical housing 912 may be transferred to the third bevel gear 1023 via the mechanical engagement between the third bevel gear 1023 and the fourth bevel gear 1033. For example, the fourth bevel gear 1033 may be located distal to the third bevel gear 1023 such that proximal movement of the fourth bevel gear 1033 is transferred to the third bevel gear 1023. However, this is not required.

A stop member 1037 may extend radially from and around the lead screw 1035 and may be vertically spaced from the fourth bevel gear 1033. The stop member 1037 may be configured to be positioned on top of or above the lower dividing wall 974a while the fourth bevel gear 1033 may be positioned below the lower dividing wall 974a of the vertical housing 912. The stop member 1037 may have an axial length that is greater than an axial length of the cut-out region 978a to prevent downwards movement of the third gear assembly 1005. A raised helical thread 1039 may protrude radially outward from an outer surface of the vertical travel lead screw 1035 between the stop member 1037 and the top end 1031 thereof.

As the vertical control knob 1007 is rotated, the first bevel gear 1011 is also rotated. The rotation of first bevel gear 1011 is translated to the third bevel gear 1023 via the second bevel gear 1019. This rotation is translated to the fourth bevel gear 1033 which in turn causes the lead screw 1035 to rotate around an axis parallel to axis 906. As the lead screw 1035 is rotated, the left body portion 1070a of the vertical slide assembly 1070 is vertically translated along a height of the lead screw 1035. The direction of travel of the left body portion 1070a may be controlled by a rotational direction of the vertical control knob 1007. For example, rotation in a first direction may cause upward movement of the left body portion 1070a while rotation in a second direction, opposite the first direction, may cause downward movement of the left body portion 1070a.

Returning to FIG. 57, the guide system 900 may further include a support shaft 1094. The support shaft 1094 may extend from a proximal end 1096 positioned within the proximal housing 916 to a distal end 1098 configured to be positioned within the lower recess 928a of the horizontal support member 910. The gear and actuation assembly 994 may be configured to extend through the first distal opening 943 of the proximal housing 916. A first stop member 1099a having an outer diameter greater than a diameter of the first distal opening 943 may be against an interior surface of the proximal housing 916. The first stop member 1099a may limit distal movement of the support shaft 1094 relative to the proximal housing 916. In some cases, the first distal opening 943 may include a transition in diameter. For example, the diameter of the first distal opening 943 may be greater adjacent to the outer surface thereof than a diameter adjacent to an inner surface of the proximal housing 916. The larger diameter region of the first distal opening 943 may be configured to receive a second stop member 1099b of the support shaft 1094. The second stop member 1099b may have an outer diameter greater than the smaller diameter of the first distal opening 943 to limit proximal movement of the support shaft 1094.

In use, the guide system 900 may be secured to an ultrasound probe using a strap (although this is not required) before or after the ultrasound probe is positioned within the anatomy. For example, for a prostate biopsy, the ultrasound probe may be positioned within the rectum. Once the guide system 900 is secured to the ultrasound probe and the ultrasound probe is positioned, the vertical housing 912 may be axially displaced along the horizontal support member 910. This may be performed by actuating the horizontal control knob 1004. Next, a vertical position of the vertical slide assembly 1070 (and thus a vertical position of the needle mount member 914 and guide needle) may be adjusted. This may be performed by actuating the vertical control knob 1007 to be move the vertical slide assembly 1070 and needle mount member 914 vertically within the channel of the vertical housing 912. Once the needle mount member 914 is at the desired height or vertical location, the pivot control knob 1032 may be actuated to adjust an angle of the guide needle.

It is contemplated that multiple samples may be taken with the needle mount member 914 at a same height. For example, the angle of the guide needle may be adjusted to obtain multiple samples within a same plane. It is further contemplated that the order of the steps described herein may be rearranged. For example, the vertical and/or angular position of the guide needle may be adjusted prior to moving the vertical housing 912 along the horizontal support member 910 towards or away from the patient's body.

Those skilled in the art will recognize that the present disclosure may be manifested in a variety of forms other than the specific embodiments described and contemplated herein. Accordingly, departure in form and detail may be made without departing from the scope and spirit of the present disclosure as described in the appended claims.

What is claimed is:

1. A prostate biopsy guide system comprising:
a horizontal slide member;
a vertical support movably coupled to the horizontal slide member;
a vertical slide member movably coupled to the vertical support;
a needle mount coupled to the vertical slide member; and
a locking mechanism coupled to the vertical slide member.

2. The prostate biopsy guide system of claim 1, wherein the horizontal slide member comprises:
a first rail;
a second rail laterally spaced from the first rail; and
a crossbar extending between the first rail and the second rail.

3. The prostate biopsy guide system of claim 2, wherein the crossbar includes a concave lower surface configured to conform to an outer surface of an ultrasound probe.

4. The prostate biopsy guide system of claim 2, wherein the horizontal slide member further comprises a probe mount positioned adjacent to a proximal region of the horizontal slide member and extending between the first rail and the second rail.

5. The prostate biopsy guide system of claim 4, wherein the probe mount includes a concave lower surface configured to conform to an outer surface of an ultrasound probe.

6. The prostate biopsy guide system of claim 4, wherein the probe mount includes a first opening and a second opening, each configured to receive an end of a strap.

7. The prostate biopsy guide system of claim 1, wherein the vertical support comprises:
a proximal face;
a distal face axially spaced from the proximal face; and
a U-shaped bracket connecting the proximal face and the distal face.

8. The prostate biopsy guide system of claim 7, wherein the vertical support further comprises:
a first laterally inwardly extending wall; and
a second laterally inwardly extending wall.

9. The prostate biopsy guide system of claim 8, wherein the vertical slide member is configured to move along the first and second laterally inwardly extending walls.

10. The prostate biopsy guide system of claim 1, further comprising an actuatable end cover coupled to the vertical support.

11. The prostate biopsy guide system of claim 10, wherein the actuatable end cover is vertically displaceable relative to the vertical support to selectively secure the vertical support relative to the horizontal slide member.

12. The prostate biopsy guide system of claim 1, wherein the vertical slide member comprises:
a proximal region configured to be coupled to the locking mechanism; and
a distal region configured to be movably coupled to the vertical support.

13. The prostate biopsy guide system of claim 12, wherein the distal region of the vertical slide member includes an elongated central aperture.

14. The prostate biopsy guide system of claim 1, wherein the needle mount comprises a tubular body defining a lumen for receiving a portion of a guide needle therethrough.

15. The prostate biopsy guide system of claim 1, wherein the locking mechanism is movable between:
a first configuration in which both vertical movement of the vertical slide member and swivel movement of the needle mount are prevented;
a second configuration in which vertical movement of the vertical slide member is prevented while allowing swivel movement of the needle mount; and
a third configuration in which both vertical movement of the vertical slide member and swivel movement of the needle mount are allowed.

16. A prostate biopsy guide system comprising:
a horizontal slide member configured to be coupled to an ultrasound probe;
a vertical support movably coupled to the horizontal slide member;
a vertical slide member movably coupled to the vertical support;
a needle mount pivotably coupled to the vertical slide member; and
a locking mechanism coupled to the vertical slide member and configured to selectively lock a vertical position of the vertical slide member and an angular position of the needle mount.

17. The prostate biopsy guide system of claim 16, wherein the locking mechanism comprises:
a handle;
a first connection member extending from the handle;
a second connection member extending from the handle;
a first swivel lock cam member coupled to the first connection member;
a second swivel lock cam member coupled to the second connection member;
a first vertical lock cam member coupled to the first connection member; and
a second vertical lock cam member coupled to the second connection member.

18. The prostate biopsy guide system of claim 17, wherein the first and second swivel lock cam members are configured to selectively engage mounting members of the needle mount to prevent swivel movement of the needle mount.

19. The prostate biopsy guide system of claim 17, wherein the first and second vertical lock cam members are configured to selectively engage a proximal face of the vertical support to prevent vertical movement of the vertical slide member.

20. A prostate biopsy guide system comprising:
a horizontal slide member configured to be coupled to an ultrasound probe;
a vertical support movably coupled to the horizontal slide member;
a vertical slide member movably coupled to the vertical support;
a needle mount pivotably coupled to the vertical slide member; and
means for selectively locking a vertical position of the vertical slide member and an angular position of the needle mount.
